(19) United States
(12) Patent Application Publication
(10) Pub. No.: US 2026/0234099 A1
(43) Pub. Date: Aug. 13, 2026

(54) Endocannabinoid ligands and related analogs

(71) Applicant: Northeastern University, Boston, MA (US)

(72) Inventors: Alexandros Makriyannis, Watertown, MA (US); Spyridon P. Nikas, Newton, MA (US); Lipin Ji, Malden, MA (US); Yingpeng Liu, Lynnfield, MA (US); Fei Tong, Cambridge, MA (US); Markos-Orestis Georgiadis, Boston, MA (US)

(21) Appl. No.: 19/155,589

(22) PCT Filed: Jan. 5, 2024
(86) PCT No.: PCT/US2024/010411

§ 371 Date: Aug. 12, 2025

Related U.S. Application Data
(60) Provisional application No. 63/484,617, filed on Feb. 13, 2023.

(51) Int. Cl. C07C 233/20; A61K 31/164; A61K 31/275; A61K 31/351; C07B 59/00; C07C 233/21; C07C 233/22; C07C 233/60; C07C 255/23; C07D 309/12

(52) U.S. Cl. CPC C07C 233/20; A61K 31/164; A61K 31/275; A61K 31/351; C07B 59/001; C07C 233/21; C07C 233/22; C07C 233/60; C07C 255/23; C07D 309/12; C07C 2601/02; C07C 2601/04; C07C 2601/08; C07C 2601/14; C07C 2601/16; C07C 2602/38

(57) ABSTRACT

Ligands that bind to and modulate (activate or deactivate) cannabinoid receptors having cannabinergic activity are described. These ligands are termed endocannabinoid analogs (“eCB analogs”) because they possess structural similarities with the endogenously produced cannabinoids (or eCB lipids). The eCB analogs comprise advantageous and unique structural features when compared to currently known, naturally occurring eCBs, as well as synthetic eCB analogs.

### RELATED APPLICATION

This application claims the benefit of U.S. Provisional Application No. 63/484,617, filed on Feb. 13, 2023. The entire teachings of the above application(s) are incorporated herein by reference.

### GOVERNMENT SUPPORT

This invention was made with government support under Grant Number DA009158 awarded by the National Institutes of Health. The government has certain rights in the invention.

### BACKGROUND

The classical cannabinoid Δ⁹-tetrahydrocannabinol (Δ⁹-THC) is the major active constituent extracted from Cannabis sativa. The effects of cannabinoids are due to an interaction with specific high affinity receptors. Presently two G_{i/o} protein coupled cannabinoid receptors, namely CB1 and CB2, have been characterized in mammals and other organisms. The CB1 receptor is very densely distributed through the central nervous system, and at lower levels in various peripheral tissues, including the myocardium, postgangliomic autonomic nerve terminals, and vascular endothelial and smooth muscle cells as well as the liver, skeletal muscle and adipose tissue (Pacher, et al., Pharmacol. Rev. (2006) 58:389-462; Batkai, et al., Circulation (2004) 110:1996-2002; Bonz, et al., J. Cardiovasc. Pharmacol. (2005) 41:657-664; Mukhopadhyay, et al., J. Am. Coll. Cardiol. (2007) 50:528-536; Rajesh, et al., Am. J. Physiol. Heart Circ. Physiol. (2007) 293:H2210-H2218; Rajesh, et al., Br. J. Pharmacol. (2008) 153:347-357; Mallat, et al. Am. J. Physiol. Gastrointest. Liver Physiol. (2008) 294:9-12; Osei-Hyiaman, et al., J. Clin. Invest. (2005) 115:1298-1305; Engeli, et al., Diabetes (2005) 54:2838-2843; Jeong, et al., Cell. Metab. (2008) 7:227-235; Pagotto, et al., Endocr. Rev. (2006) 27:73-100; Cota, et al., J. Clin. Invest. (2003) 112:423-431).

The CB2 receptor is present in immune and hematopoietic cells and recently has also been identified in the brain, myocardium, liver, and human coronary endothelial and smooth muscle cells (Van Sickle, et al., Science (2005) 310:329-332; Gong, et al., Brain Res. (2006) 1071:10-23; Mukhopadhyay, et al., J. Am. Coll. Cardiol. (2007) 50:528-536; Mallat, et al., Am. J. Physiol. Gastrointest. Liver Physiol. (2008) 294:9-12; Rajesh, et al. Am. J. Physiol. Heart Circ. Physiol. (2007) 293:H2210-2218; Rajesh, et al., Br. J. Pharmacol. (2008) 153:347-357).

Some compounds (cannabinergic ligands) can bind to and modulate (activate or deactivate) the CB1 and/or CB2 receptors in an individual or animal. In vitro methods for assaying the ability of a compound to bind to and modulate CB1 and/or CB2 receptors are known. Results from the in vitro binding and functional assays (see definitions section below) correlate with and predict the in vivo ability of that compound to bind to CB1 and/or CB2 receptors and modulate their function(s). When introduced in an individual or animal some of these cannabinergic ligands can bind to and modulate (activate or deactivate) the CB1 and/or CB2 receptors. Examples of some cannabinergic ligands include N-arachidonoyl ethanolamine (anandamide, AEA) and 2-arachidonoylglycerol (2-AG) (both endogenous ligands for the cannabinoid CB1 and CB2 receptors), (−)-Δ⁹-tetrahydrocannabinol (Δ⁹-THC, the principal bioactive constituent of cannabis preparations and exogenous ligand for the cannabinoid CB1 and CB2 receptors) and other synthetic cannabinergic analogs. For reviews see: Pavlopoulos et. al., Curr. Pharm. Des. (2006) 12:1751-1769 and Iliopoulos-Tsoutsouvas et al. (2020) ‘Natural Compounds and Synthetic Drugs to Target Type-1 Cannabinoid (CB1) Receptor’ in Maccarrone M. editor, New Tools to Interrogate Endocannabinoid Signaling, RSC (2020).

Ligands for the CB1/CB2 cannabinoid receptors, such as AEA, 2-AG, (−)-Δ⁹-tetrahydrocannabinol can bind to and modulate (activate or deactivate) the CB1/CB2 cannabinoid receptors and thereby provide a physiological effect in an individual or animal that is useful to treat a condition in that individual or animal. Conditions that may be treated by modulation of the CB1/CB2 cannabinoid receptors include for example: pain; central pain; peripheral pain; neuropathic pain; neuropathy; inflammatory pain; neurodegenerative diseases including multiple sclerosis, Parkinson's disease, Huntington's chorea, Alzheimer's disease and amyotrophic lateral sclerosis; mental disorders such as schizophrenia and depression; mood disorders; addiction disorders; memory disorders; gastrointestinal motility disorders such as irritable bowel syndrome and diarrhea; dyskinesia; migraine; osteoporosis, osteoarthritis; high blood pressure disease or hypertension; peripheral vascular disease; coronary artery disease; abnormal heart rate; cardiac insufficiency; pulmonary hypertension; ocular hypertension or glaucoma; to prevent or reduce endotoxic shock and hypotensive shock; to modulate appetite; to modulate the immune system; to modulate fertility; to prevent or reduce diseases associated with motor dysfunction such as Tourette's syndrome; to prevent or reduce inflammation; to provide neuroprotection; to produce peripheral vasodilation; to treat epilepsy; to treat nausea such as associated with cancer chemotherapy; AIDS wasting syndrome; HIV and HIV related disorders; autism and autism spectrum disorder; to treat several types of cancer as well as other ailments in which the cannabinoid system is implicated.

### SUMMARY

Novel cannabinoid ligands represented by the general formulas I, II, III, IV and V and methods for preparation and use are presented. The disclosed compounds can bind to and modulate the cannabinoid CB1/CB2 receptors and thus, they are specific ligands for these receptors. The disclosed compounds, when administered in a therapeutically effective amount to an individual or animal, result in a sufficiently high level of that compound in the individual or animal to cause a physiological response. The physiological response may be useful to treat a number of physiological conditions.

Moreover, as detailed in the invention, the carboxylic acid/ester derivatives/precursors to eCB analogs disclosed herein, have high potential to be biologically active on their own, or after their conversion by oxidative enzymes including but not limited to P450, cyclooxygenases and lipoxygenases. Therefore, the carboxylic acid/ester derivatives/precursors of the eCB analogs represented by the general formulas I, II, IV and V bellow, where A₁ or A₂, A₄ or A₅ is COOH (carboxylic acid group) or A₁ or A₂, or A₄, or A₅ is COOR″ (carboxylic ester group) are also included in this invention.

In one aspect, a compound of formula I or a pharmaceutically acceptable salt thereof, including all stereoisomers and enantiomers is disclosed:

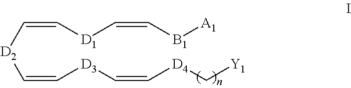

Wherein:
A₁ is selected from

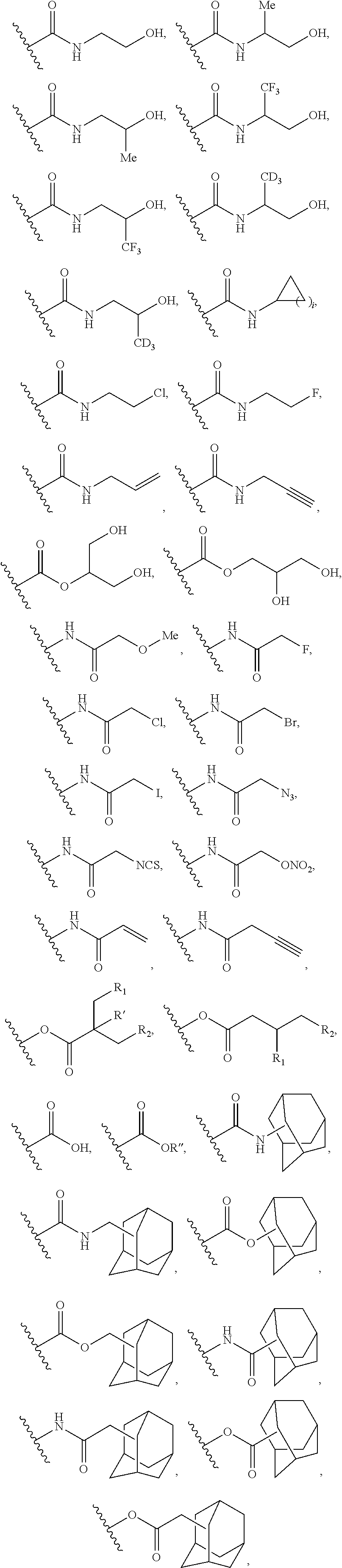

i is an integer from 1 to about 5,
R₁ and R₂ are independently selected from —H, -D, —Cl, —Br, —F, —OH, -Me, —CHF₂, —CH₂F, —CF₃,
R′ is selected from —H, -D and —CH₃,
R″ is -alkyl,
B₁ is selected from

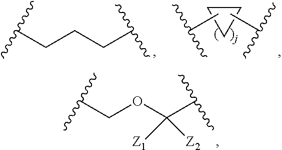

j is an integer from 1 to about 4,
Z₁, Z₂, independently selected from —H, -D, —F, -Me, —CF₃,
D₁, D₂, D₃ and D₄ are each independently selected from —CH₂—, —CF₂—, —CD₂- and

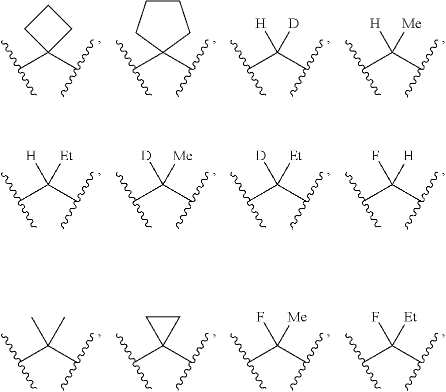

provided that at least one of D₁ or D₂ or D₃ or D₄ in general formula I is a chiral group selected from

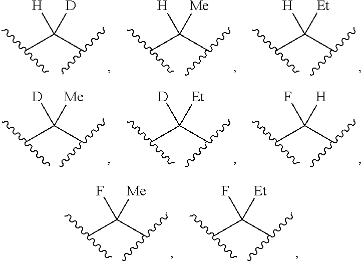

n is an integer from 1 to about 7,
Y₁ is selected from -D, —CD₃, —F, —I, —Cl, —Br, —CF₃, —CN, —NO₂, —SO₂F, —ONO₂, —OH, —SH, —NH₂, —(NH)-Alkyl, —N(Alkyl)₂, -heterocyclic ring, -carbocyclic ring, -Ph, —SPh, —SOPh, —SO₂Ph and

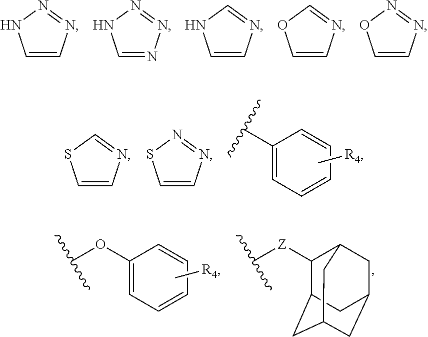

unless otherwise specifically denoted, each of the aforementioned groups is connected to the main structure from any possible position given that it is fulfilling the valency requirements.
R₄ is —F, —CN or —CF₃,
Z is —NH—, —CH₂NH—, —NHCONH—, —COO—, —COOCH₂— or —CH₂—,
wherein the compound cannot be methyl (5Z,8Z,11Z,14Z)-20-hydroxy-13-methylicosa-5,8,11,14-tetraenoate.

In another aspect, a compound, or a pharmaceutically acceptable salt thereof, including all stereoisomers and enantiomers, is disclosed, wherein the compound is selected from the group consisting of:

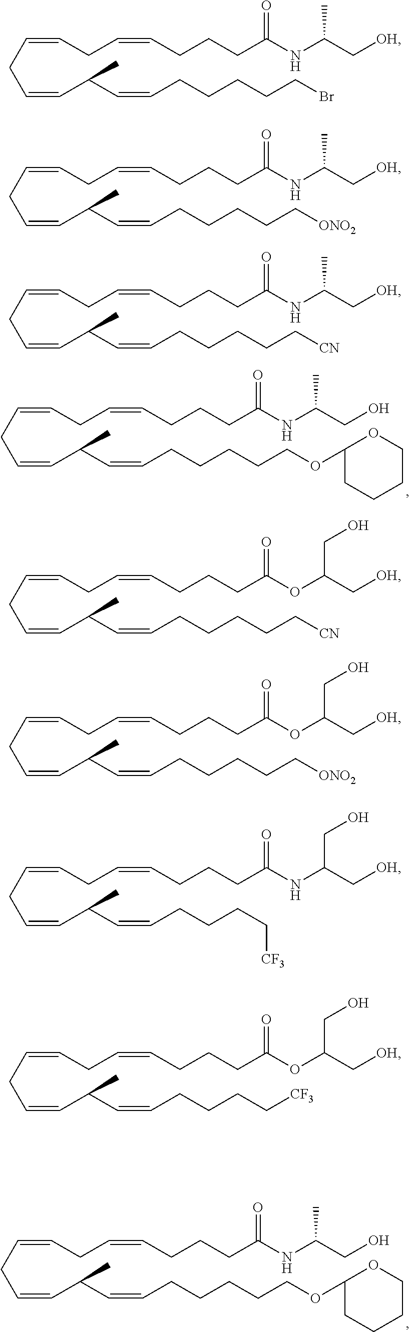

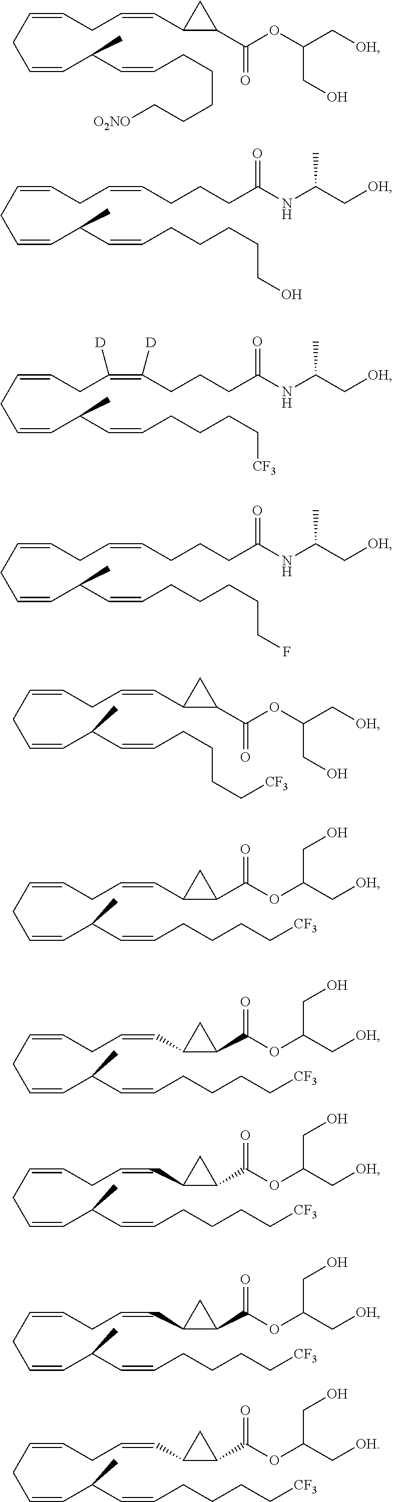

In another aspect, a compound of formula II or a pharmaceutically acceptable salt thereof, including all stereoisomers and enantiomers is disclosed:

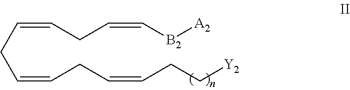

wherein:
A₂ is selected from

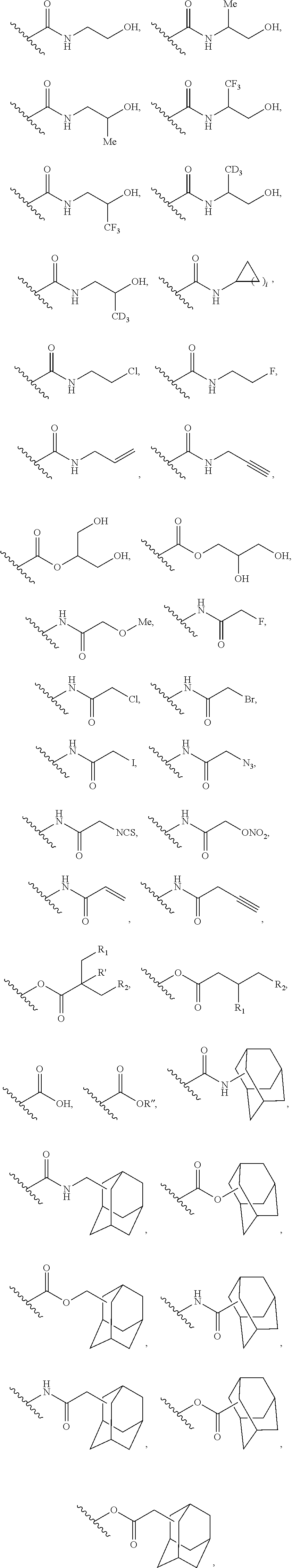

i is an integer from 1 to about 5,
R₁ and R₂ are independently selected from —H, -D, —Cl, —Br, —F, —OH, -Me, —CHF₂, —CH₂F, —CF₃,
R′ is selected from —H, -D and —CH₃,
R″ is -alkyl,
B₂ is selected from

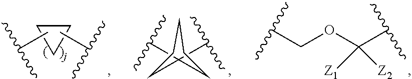

j is an integer from 1 to about 4,
Z₁, Z₂, independently selected from -D, —F, -Me, —CF₃,
n is an integer from 1 to about 7,
Y₂ is selected from -Alkyl, —H, -D, —F, —I, —Cl, —Br, —CF₃, —CN, —NCS, —N₃—NO₂, —SO₂F, —ONO₂, —OH, —SH, —NH₂, —(NH)-Alkyl, —N(Alkyl)₂, -heterocyclic ring, -carbocyclic ring, -Ph, —OPh, —SPh, —SOPh, —SO₂Ph and

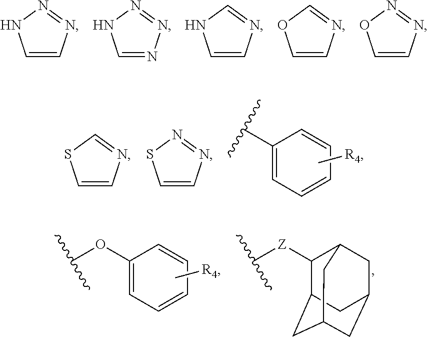

unless otherwise specifically denoted, each of the aforementioned groups is connected to the main structure from any possible position given that it is fulfilling the valency requirements,
R₄ is selected from —F, —CN, —CF₃,
Z is —NH—, —CH₂NH—, —COO—, —COOCH₂— or —CH₂—.

In another aspect, a compound or a pharmaceutically acceptable salt thereof, including all stereoisomers and enantiomers, is disclosed, wherein the compound is selected from the group consisting of:

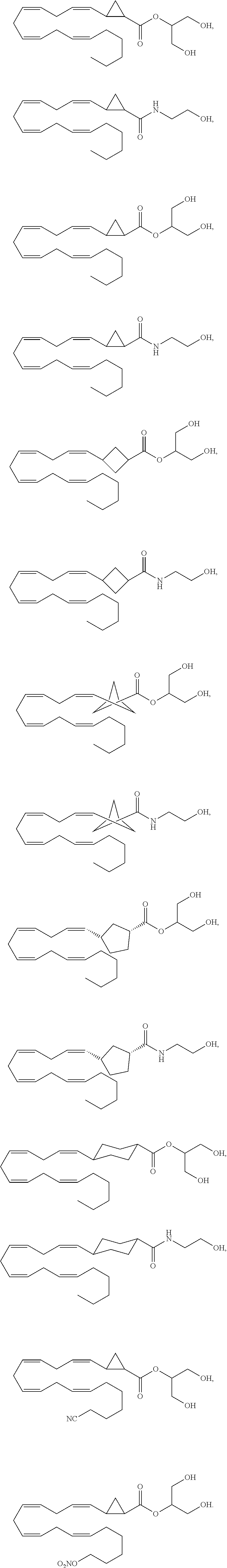

In another aspect, a compound of formula II or a pharmaceutically acceptable salt thereof, including all stereoisomers and enantiomers is disclosed:

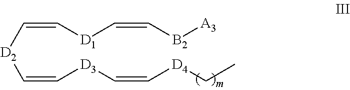

wherein:
A₃ is selected from

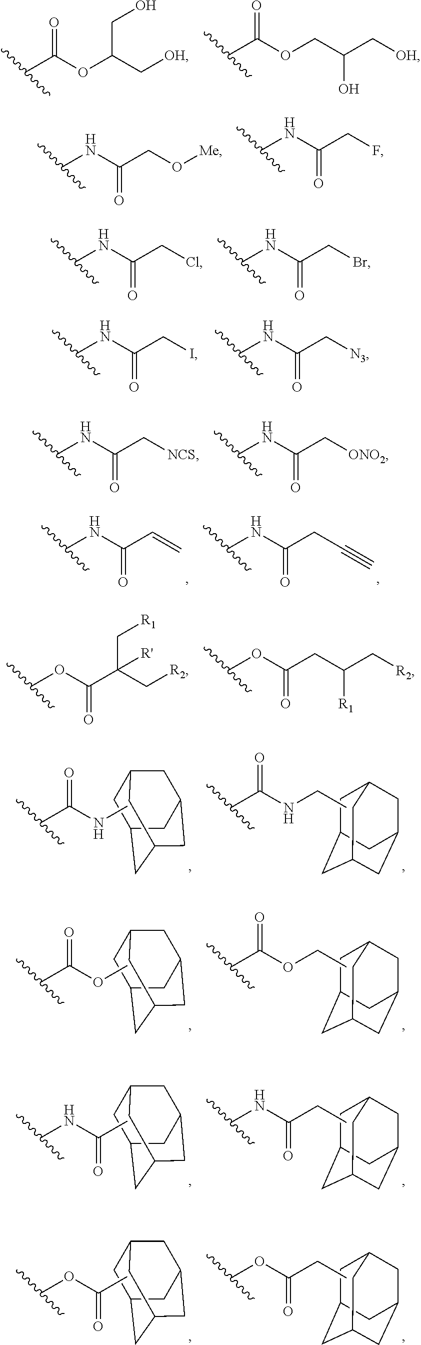

R₁ and R₂ are independently selected from —H, -D, —Cl, —Br, —F, —OH, -Me, —CHF₂, —CH₂F, —CF₃,
R′ is selected from —H, -D and —CH₃,
B₃ is selected from

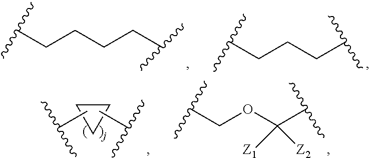

j is an integer from 1 to about 4,
Z₁, Z₂, independently selected from —H, -D, —F, -Me, —CF₃,
D₁, D₂, D₃ and D₄ are each independently selected from —CH₂—, —CF₂—, —CD₂- and

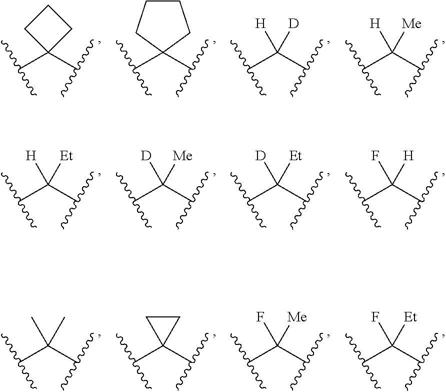

provided that at least one of D₁ or D₂ or D₃ or D₄ in general formula III is a chiral group selected from

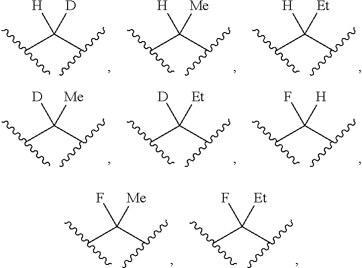

m is an integer from 0 to about 6.

In another aspect, a compound, or a pharmaceutically acceptable salt thereof, including all stereoisomers and enantiomers, is disclosed, wherein the compound is selected from the group consisting of:

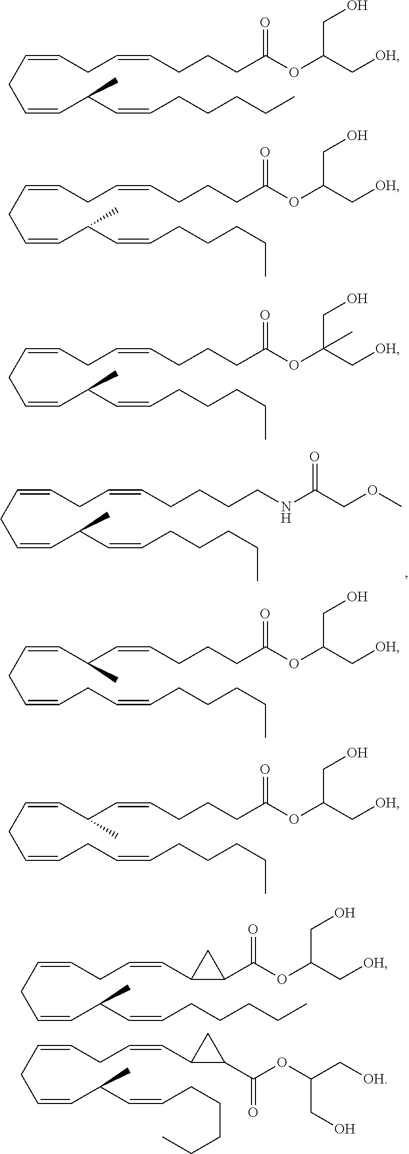

In another aspect, a compound of formula IV or a pharmaceutically acceptable salt thereof, including all stereoisomers and enantiomers is disclosed:

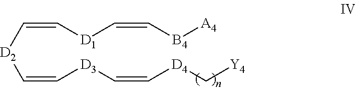

wherein:
A₄ is selected from

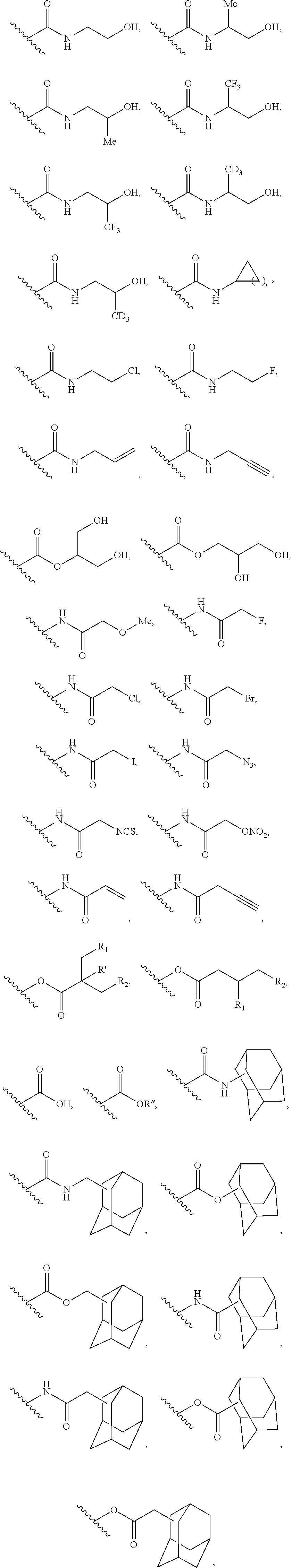

i is an integer from 1 to about 5,
R₁ and R₂ are independently selected from —H, -D, —Cl, —Br, —F, —OH, -Me, —CHF₂, —CH₂F, —CF₃,
R′ is selected from —H, -D and —CH₃,
R″ is -alkyl,
B₄ is selected from

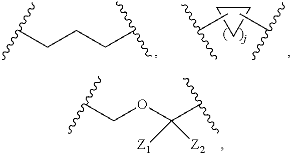

j is an integer from 1 to about 4,
Z₁, Z₂, independently selected from —H, -D, —F, -Me, —CF₃,
D₁, D₂, D₃ and D₄ are each independently selected from —CH₂—, —CF₂—, —CD₂- and

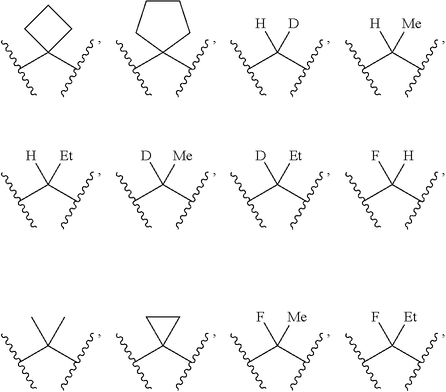

provided that at least one of D₁ or D₂ or D₃ or D₄ is a cycloalkyl group selected from:

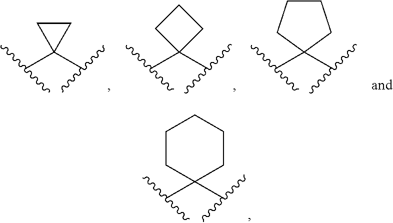 [SMILES: *C1(*)CC1 ; *C1(*)CCC1 ; *C1(*)CCCC1 ; *C1(*)CCCCC1]

Y₄ is selected from -Alkyl, —H, -D, —F, —I, —Cl, Br, —CF₃, —CN, —NCS, —N₃—NO₂, —SO₂F, —ONO₂, —OH, —SH, —NH₂, —(NH)-Alkyl, —N(Alkyl)₂, -heterocyclic ring, -carbocyclic ring, -Ph, —OPh, —SPh, —SOPh, —SO₂Ph and

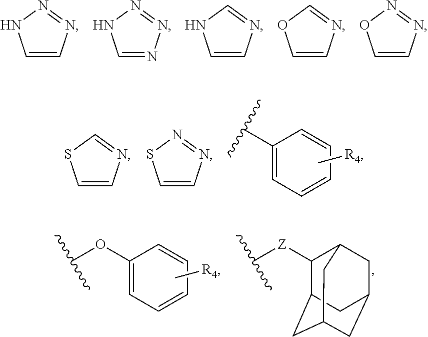

unless otherwise specifically denoted, each of the aforementioned groups is connected to the main structure from any possible position given that it is fulfilling the valency requirements,
R₄ is selected from —F, —CN, —CF₃,
Z is —NH—, —CH₂NH—, —NHCONH—, —COO—, —COOCH₂— or —CH₂—.

In another aspect, a compound, or a pharmaceutically acceptable salt thereof, including all stereoisomers and enantiomers, is disclosed, wherein the compound is selected from the group consisting of:

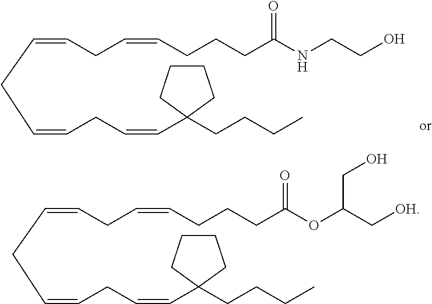

In another aspect, a compound of formula V or a pharmaceutically acceptable salt thereof, including all stereoisomers and enantiomers is disclosed:

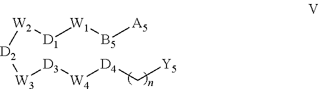

wherein:
A₅ is selected from

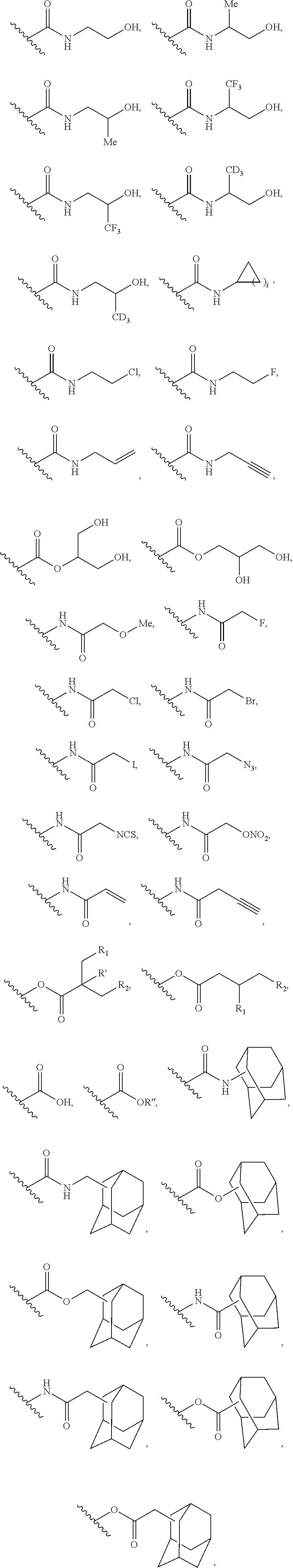

R₁, R₂ are independently selected from —H, -D, —Cl, —Br, —F, —OH, -Me, —CHF₂, —CH₂F, —CF₃,
R′ is selected from —H, -D and —CH₃,
R″ is -alkyl,
i is an integer from 1 to about 5,
B₅ is selected from

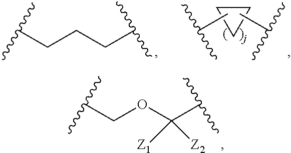

j is an integer from 1 to about 4,
Z₁, Z₂, independently selected from —H, -D, —F, -Me, —CF₃,
W₁, W₂, W₃ and W₄ are independently selected from ═, ≡ or a π electron system selected from the groups shown below

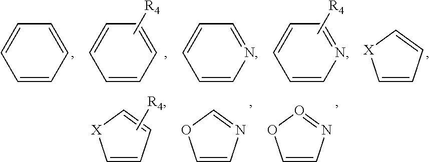

provided that the general structure V encompasses at least one of the aforementioned π electron systems and each of the π electron systems is connected to the main structure in any possible position given that it is fulfilling the valency requirements,
R₄ is selected from -D, —F, —CF₃, —CN,
X is selected from CH₂, NH, O, S,
D₁, D₂, D₃ and D₄ are independently selected from —CH₂—, —CF₂—, —CD₂- and

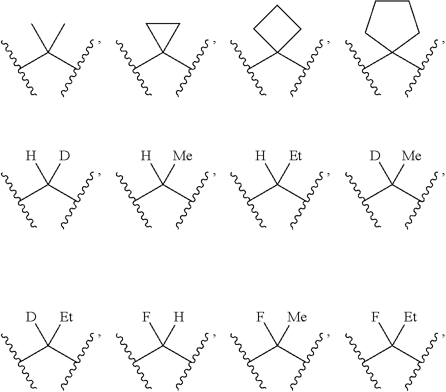

n is an integer from 1 to about 7,
Y₅ is selected from -Alkyl, —H, -D, —F, —I, —Cl, Br, —CF₃, —CN, —NCS, —N₃—NO₂, —SO₂F, —ONO₂, —OH, —SH, —NH₂, —(NH)-Alkyl, —N(Alkyl)₂, -heterocyclic ring, -carbocyclic ring, -Ph, —OPh, —SPh, —SOPh, —SO₂Ph and

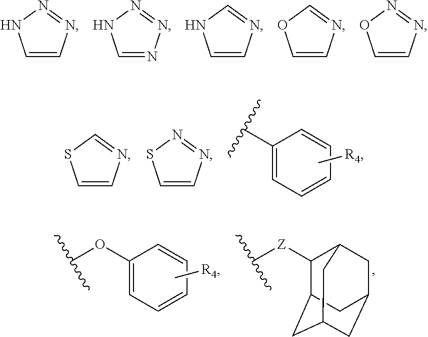

unless otherwise specifically denoted, each of the aforementioned groups is connected to the main structure from any possible position given that it is fulfilling the valency requirements,
R₄ is selected from —F, —CN, —CF₃,
Z is —NH—, —CH₂NH—, —NHCONH—, —COO—, —COOCH₂— or —CH₂—.

In another aspect, a compound, or a pharmaceutically acceptable salt thereof, including all stereoisomers and enantiomers, is disclosed, wherein the compound is selected from the group consisting of:

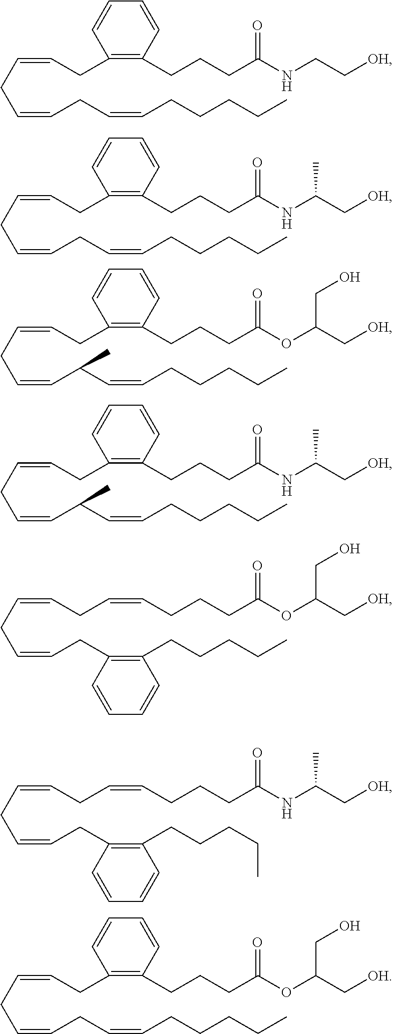

In another aspect, a pharmaceutical composition comprising a therapeutically effective amount of a compound of any preceding claim or a pharmaceutically acceptable salt thereof and a pharmaceutically acceptable carrier is disclosed.

In another aspect, method of treating a condition in a subject in need thereof is disclosed, the method comprising administering to the subject a therapeutically effective amount of a compound or pharmaceutically acceptable salt or a pharmaceutical composition disclosed herein; wherein the condition is selected from the group consisting of pain; central pain; peripheral pain; neuropathic pain; neuropathy; inflammatory pain; neurodegenerative diseases including multiple sclerosis, Parkinson's disease, Huntington's chorea, Alzheimer's disease and amyotrophic lateral sclerosis; mental disorders such as schizophrenia and depression; mood disorders; addiction disorders; memory disorders; gastrointestinal motility disorders such as irritable bowel syndrome and diarrhea; dyskinesia; migraine; osteoporosis, osteoarthritis; high blood pressure disease or hypertension; peripheral vascular disease; coronary artery disease; abnormal heart rate; cardiac insufficiency; pulmonary hypertension; ocular hypertension or glaucoma; endotoxic shock; hypotensive shock; appetite disorders; immune system disorders; fertility disorders; diseases associated with motor dysfunction such as Tourette's syndrome; inflammation; neurological disorders; epilepsy; nausea; AIDS wasting syndrome; HIV and HIV related disorders; autism and autism spectrum disorder; cancer.

In another aspect, a method of stimulating (activating) a cannabinoid receptor in a subject is disclosed, the method comprising: administering to the subject an effective amount of a compound or pharmaceutically acceptable salt or a pharmaceutical composition disclosed herein.

In another aspect, a method of selectively stimulating a CB1 cannabinoid receptor in subject is disclosed (see the definitions section and data Tables for examples of novel compounds disclosed herein and their selectivity for CB1 receptors), the method comprising: administering to the subject an effective amount of a compound or pharmaceutically acceptable salt or a pharmaceutical composition disclosed herein.

In another aspect, a method of selectively stimulating a cannabinoid receptor either in the CNS or in the periphery of a subject is disclosed (for more details see the definitions section and especially the section related to polar characteristics of the novel disclosed compounds in Table 6), the method comprising administering to the subject an effective amount of a compound or pharmaceutically acceptable salt or a pharmaceutical composition disclosed herein.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A is a graph providing data of the % inhibition of FSK stimulated cAMP for compound 5.4.3d for rCB1.

FIG. 1B is a graph providing data of the % inhibition of FSK stimulated cAMP for the standard full agonist CP-55,940 for hCB2.

FIG. 2A is a graph providing data of the % inhibition of FSK stimulated cAMP for compound 6.1.1 for hCB2.

FIG. 2B is a graph providing data of the % inhibition of FSK stimulated cAMP for the standard full agonist CP-55,940 for hCB2.

### DETAILED DESCRIPTION

A description of example embodiments follows.

The present disclosure relates generally to chemical compounds with cannabinergic activity; to pharmaceutical compositions comprising the compounds; and to methods of preparing said compounds as well as their uses. The present disclosure is also related to novel compounds disclosed herein for methods of: treating diseases, disorders, or conditions; imaging; selectively stimulating a cannabinoid receptor; and assessing a compound as a CB modulator. The invented cannabinoid compounds are structurally not related to plant derived cannabinoids or similar synthetic derivatives such as THC or CBD or Nabilone. More specifically, the present disclosure is concerned with novel ligands that bind to and modulate (activate or deactivate) cannabinoid receptors. The novel ligands disclosed herein are termed “endocannabinoid analogs” (“eCB analogs”) because they possess structural similarities with the endogenously produced cannabinoids, or eCB lipids, namely anandamide (AEA) and 2-arachidonoyl glycerol (2-AG). The disclosure is also concerned with the synthetic precursors of these eCB analogs. The synthetic precursors are carboxylic acid or ester derivatives isolated during the synthesis of the eCB analogs which have the potential to be biologically active on their own. The novel eCB analogs and their precursors disclosed herein comprise advantageous and unique structural features when compared to currently known, naturally occurring eCBs, as well as other synthetic eCB analogs. These structural features include a) chiral centers, b) π-electron rings (for example, phenyl) that resemble the double bonds of the endocannabinoids AEA and 2-AG, c), groups with enhanced polarity (e.g., CN or ONO₂), d) cycloalkyl groups replacing linear alkyl groups of the AEA or 2-AG structure, or cycloalkyl groups added at key positions of the AEA or 2-AG structure to constrain their conformational properties, and e) combinations thereof. The disclosure is also concerned with pharmaceutical preparations employing these ligands and methods of administering therapeutically effective amounts of the ligands to provide a physiological effect.

The advantageous and unique structural features are incorporated injudiciously chosen positions within the endocannabinoid structural prototype in order to maintain or even enhance the pharmacodynamic and pharmacokinetic performance of the novel eCB analogs when compared to the endogenous cannabinoid lipids or to other synthetic eCB analogs reported in the literature (see for example, biological testing results (Tables 1-5 and 7) of the novel disclosed compounds and how they compare to the endocannabinoids AEA and 2-AG as well as to the earlier reported compounds such as AMG315). Information regarding the meaning of the Ki and EC50 values, shown on Tables 1-5 and 7, is provided in the definitions section. As presented herein, the biological data indicate that the novel endocannabinoid analogs disclosed herein exhibit enhanced pharmacodynamic profiles (for example, higher binding affinities and/or higher agonist potencies for the cannabinoid receptors).

Particularly worthy of note is the incorporation of n-electron systems that replace double bonds of the eCBs. The incorporation of such n-electron systems in place of double bonds is disclosed herein for the first time and is the first reported successful modification of the tetraolefinic moiety of the eCB template. The novel structural modification in the tetraolefinic portion of the eCB template maintains or even increases the binding affinity of the ligand for CB receptors (for example, as shown in Table 5, the analogs 4.3.4, 4.3.5 and 4.5.3 exhibit substantially higher binding affinities for the cannabinoid CB1 receptor when compared to the endogenous AEA). In addition to the enhancement of the pharmacodynamic profile of the phenyl ring-containing eCB analogs, other benefits of the structural modification include: a) conformational restriction of the flexible lipid-like structure imposed by the presence of the flat and rigid phenyl ring which, thus, increases the drug-like profile of the molecule, and b) avoidance of metabolic epoxidation of the double bond by oxidases/epoxygenases (a well know liability of molecules carrying double bonds). Earlier reported attempts to modify the tetraolefinic moiety of the eCBs have generally failed.

Additionally, in Table 6, the polar characteristics of the novel disclosed compounds are compared to the endogenous ligands AEA and 2-AG, and to the earlier reported AMG315. Based on the calculated c Log P and tPSA values (see also definitions section) the novel compounds appear to be more polar which is a highly sought after characteristic in the field of drug discovery.

The present disclosure is also concerned with pharmaceutical preparations employing these novel endocannabinoid analogs and methods of administering therapeutically effective amounts of these compounds to provide a physiological effect.

The invented compounds, when administered in a therapeutically effective amount to an individual or animal, result in a sufficiently high level of that compound in the individual or animal to cause a physiological response. The physiological response may be useful to treat a number of physiological conditions. As detailed in the invention, in addition to chemical compounds with cannabinergic activity the carboxylic acid derivatives/precursors to eCB analogs disclosed herein, have high potential to be biologically active on their own, or after their conversion by oxidative enzymes including but not limited to P450, cyclooxygenases and lipoxygenases. Therefore, the carboxylic acid derivatives/precursors of the eCB analogs represented by the general formulas I, II, IV and V where A₁ or A₂, or A₄ or A₅ is COOH (carboxylic acid group) or A₁ or A₂, or A₄, or A₅ is COOR″ (carboxylic ester group) or each equivalent COOR″ (carboxylic acid ester group) are also included in this disclosure.

As detailed under the definitions section, the carboxylic acid derivatives/precursors to eCB analogs disclosed herein (for examples see synthetic schemes below) have high potential to be biologically active on their own, or after their conversion by oxidative enzymes including but not limited to P450, cyclooxygenases and lipoxygenases. These carboxylic acid precursors of the eCB analogs disclosed herein, or their oxidative metabolites, have high potential to modulate CB receptors or other biological targets (examples of which are given in definitions section). Therefore, the carboxylic acid derivatives/precursors of the eCB analogs represented by the general formulas I, II, IV and V where A₁ or A₂, or A₄, or A₅ is COOH (carboxylic acid group) or A₁ or A₂, or A₄, or A₅ is COOR″ (carboxylic ester group) or each equivalent COOR″ (carboxylic acid ester group) are also included in the present disclosure.

Embodiments or aspects of the disclosure described as a method should also be understood to include, but are not limited to, purpose-limited products, purpose-limited processes, first or subsequent “medical use” embodiments or aspects of the disclosure, a compound for treatment of a disease or condition, use of a compound for treatment of a disease or condition, or “Swiss use” of a compound in the manufacture of a medicament for treatment of a disease or condition. A compound in any one of these formats is understood to mean any one or combination of the compounds disclosed in the present disclosure or in combination with known compounds to achieve the same function or response.

Unless otherwise defined, all technical and scientific terms used herein have the same meaning as commonly understood by one of ordinary skill in the art to which this disclosure belongs. Although methods and materials similar or equivalent to those described herein can be used in accordance with the present disclosure, suitable methods and materials are described below. Those skilled in the art to which the present disclosure pertains may make modifications resulting in other embodiments or aspects employing principles of the present disclosure without departing from its spirit or characteristics, particularly upon considering the foregoing teachings. All publications, patent applications, patents, and other references mentioned herein are incorporated by reference in their entirety. In case of conflict, the present specification, including definitions, will control. In addition, the materials, methods, embodiments, aspects, and examples are illustrative only and not intended to be limiting. Other features and advantages of the present disclosure will be apparent from the following detailed description, and from the claims. While the present disclosure includes references to particular embodiments and aspects, modifications of structure, method, materials and the like apparent to those skilled in the art still fall within the scope as claimed.

### A. DEFINITIONS

The compounds of this disclosure include any and all possible isomers, stereoisomers, enantiomers diastereomers, tautomers, pharmaceutically-acceptable salts, and solvates thereof. Thus, the terms “compound” and “compounds” as used in this disclosure refer to the compounds of this disclosure and any of all possible isomers, stereoisomers, enantiomers diastereomers, tautomers, pharmaceutically-acceptable salts, and solvates thereof.

As used herein, the term “about” or “approximately” refers to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In some embodiments, the terms “about” or “approximately” when preceding a numerical value indicates the value plus or minus a range of 15%, 10%, 5%, or 1%.

As used herein, the articles “a,” “an,” and “the” are used herein to refer to one or to more than one (i.e., to at least one) of the grammatical object of the article. By way of example, “an element” can mean one element or more than one element.

Also, the use of “or” means “and/or” unless stated otherwise. Similarly, “comprise,” “comprises,” “comprising” “include,” “includes,” and “including” are interchangeable and not intended to be limiting.

Unless otherwise specifically defined, “alkyl” refers to a linear or branched hydrocarbon radical which may be fully saturated, mono- or polyunsaturated and can include divalent radicals, having from 1 to about 15 carbon atoms if it is saturated, or from 2 to about 15 carbon atoms if it is unsaturated. Examples for saturated hydrocarbon radicals include, but are not limited to, groups such as methyl (Me), ethyl (Et), n-propyl, isopropyl, n-butyl, t-butyl, isobutyl, sec-butyl, homologs and isomers of, for example, n-pentyl, n-hexyl, n-heptyl, n-octyl, 1,1-dimethyl-heptyl, 1,2-dimethyl-heptyl, and the like. An unsaturated alkyl group includes one or more double bonds, triple bonds or combinations thereof. Examples of unsaturated alkyl groups include but are not limited to, vinyl, propenyl, isopropenyl, crotyl, 2-isopentenyl, allenyl, butenyl, butadienyl, pentenyl, pentadienyl, 3-(1,4-pentadienyl), hexenyl, hexadienyl, ethynyl, propynyl, butynyl, and higher homologs and isomers. The term “divalent alkyl radicals” unless otherwise specifically defined refers to the general formula: -alkyl-. The term “C₁₋ₙ-alkyl” refers to an alkyl having from 1 to about n carbon atoms.

Unless otherwise specifically defined, “alkylamino” refers to the general formula —(NH)-alkyl.

Unless otherwise specifically defined, “di-alkylamino” refers to the general formula —N-(alkyl)₂. Unless otherwise specifically limited di-alkylamino includes cyclic amine compounds such as piperidine, piperazine, azetidine, pyrrolidine, morpholine and their derivatives.

Unless otherwise specifically defined, “heterocyclic moiety”, “heterocyclic” or “heterocyclic ring” refers to a saturated ring structure having about 3 to about 8 ring members that has carbon atoms and one or more heteroatoms, including oxygen, nitrogen and/or sulfur, as ring atoms. The term “heterocyclic moiety”, “heterocyclic” or “heterocyclic ring” can include “divalent radicals”. The term “divalent heterocyclic radicals” unless otherwise specifically defined refers to the general formula: -heterocyclic-. Examples of heterocyclic moieties include but are not limited to, oxetane, thietane, azetidine, diazetidine, tetrahydrofuran, tetrahydropyran, thiolane, pyrrolidine, dioxolane, oxathiolane, imidazolidine, dioxane, piperidine, morpholine, piperazine, and their derivatives.

The phrase “pharmaceutically acceptable” is employed in this disclosure to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The terms “salt” and “salts”, as employed in the disclosure, denotes acidic and/or basic salts formed with inorganic and/or organic acids and bases. Salts further include, by way of example only, salts of non-toxic organic or inorganic acids, such as halides, such as, chloride and bromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate, and the like.

As used herein “pharmaceutically acceptable salt” refers to any salt of a compound disclosed herein which retains its biological properties and which is not toxic or otherwise undesirable for veterinary, or pharmaceutical use. Such salts may be derived from a variety of organic and inorganic counter-ions known in the art. Such salts include: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2 naphthalenesulfonic, 4-toluenesulfonic, camphoric, camphorsulfonic, 4 methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid, and like acids.

In certain embodiments, the disclosed compounds are isolated from a naturally occurring or synthetic material. In some embodiments, the isolated compound is contemporaneously or subsequently “purified” or “substantially purified”. As used herein a “purified” or “substantially purified” compound means a compound that has been processed to a desired purity. A person of ordinary skill can establish the desired purity for a use and method to achieve that purity without undue effort. The purified compound may be used in any disclosed embodiment.

As used herein a “therapeutically effective amount” of a compound, is the quantity of a compound which, when administered to an individual or animal, results in a discernible physiological effect in the individual or animal. The compounds disclosed herein, and pharmaceutically acceptable salts thereof, have pharmacological properties when administered in therapeutically effective amounts for providing a physiological effect useful to treat a number of physiological conditions. Typically, a “therapeutically effective amount” of a compound is believed to range from about 5 mg/day to about 1,000 mg/day.

A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient.

As used herein, an “individual” refers to a human. An “animal” refers to, for example, veterinary animals, such as dogs, cats, horses and the like, and farm animals, such as cows, pigs and the like.

As used herein, “treatment”, “treat”, and “treating” refer to reversing, alleviating, mitigating, or slowing the progression of, or inhibiting the progress of, a disorder, a condition, or disease or symptoms associated with such disorder, condition, or disease, and as described in more detail herein.

As used in chemical formulas herein, when an integer is 0, the structural portion modified by that integer is absent and the adjacent subunits are directly connected.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “composition” as used herein is intended to encompass a product comprising specific ingredients in specified amounts, as well as any product which results, directly or indirectly, from combination of the specified ingredients in the specified amounts.

The compositions of the disclosure may be alternately formulated to comprise, consist of, or consist essentially of, any appropriate components herein disclosed. The compositions of the disclosure may additionally, or alternatively, be formulated so as to be devoid, or substantially free, of any components, materials, ingredients, adjuvants or species used in the prior art compositions or that are otherwise not necessary to the achievement of the function and/or objectives of the present disclosure.

The compounds of the present disclosure may have unnatural ratios of atomic isotopes at one or more of their atoms. For example, the compounds may be labeled with isotopes, such as deuterium tritium carbon-11, carbon-14, iodine-123, iodine-125 or fluorine-18. The present disclosure encompasses all isotopic variations of the described compounds, whether radioactive or not.

Currently, it is very well known that the endocannabinoids AEA and 2-AG are hydrolyzed to the respective carboxylic acid, namely, arachidonic acid, upon the action of amidases (e.g., FAAH), lipases (e.g., MGL) or esterases. It is also very well known that the respective endocannabinoid carboxylic acid (arachidonic acid) is bioactive on its own and, also, upon further biotransformation (induced by oxidative enzymes, e.g., COX-2, LOX, P450) leads to biologically active compounds that act either on CB receptors or on other targets. Examples are, but, not limited to: a) the lipoxygenase product Lipoxin A4 which is an allosteric modulator at CB1 receptor as disclosed in Pamplona et al. (2012), PNAS, 109:51, 21134-21139 and in Iliopoulos-Tsoutsouvas et al. (2020) ‘Natural Compounds and Synthetic Drugs to Target Type-1 Cannabinoid (CB1) Receptor’ in Maccarrone M. editor, New Tools to Interrogate Endocannabinoid Signaling; RSC (2020), b) Prostaglandins that target proteins different than CB receptors, c) epoxy-arachidonic acids that target TRPV channels (as disclosed in Nilius B. et al. (2004), Nature, 424). The 13-Methylarachidonic acid which is the respective carboxylic acid metabolite of the 13-methyl 2-AG analogs (5.4.3c, 5.4.3d, Table 3) which, as we disclosed in Kudalkar, S. N. et al., (2015), J. Biol. Chem. 7897, behaves as an allosteric modulator at COX-2. In conclusion, the disclosed eCB analogs and pharmaceutically acceptable salts thereof could be metabolized by hydrolytic enzymes such as amidases, or lipases or esterases into the body of an individual or animal to give the corresponding carboxylic acids (general formulas I, if, IV and V where A₁ or A₂, or A₄ or A₅ is COOH (carboxylic acid group) or A₁ or A₂, or A₄ or A₅ is a COOR″ (carboxylic acid ester group which is biologically and/or chemically, equivalent to carboxylic acid group, see for example, definition of prodrugs below). As discussed above, these carboxylic acid/carboxylic acid ester derivatives/precursors to eCB analogs disclosed herein, have high potential to be biologically active by their own, or after conversion by hydrolytic and/or oxidative enzymes including but not limited to P450, cyclooxygenases and lipoxygenases. The corresponding carboxylic acids/esters or their oxidative metabolites could target CB receptors or other biological targets (examples of which are given above). Therefore, the carboxylic acid/ester derivatives/precursors of the eCB analogs represented by the general formulas I, II, IV and V, where A₁ or A₂, or A₄ or A₅ is COOH (carboxylic acid group) or A₁ or A₂, or A₄, or A₅ is COOR″ (carboxylic ester group) are also included in this invention.

The disclosed compounds, and pharmaceutically acceptable salts thereof may be used to prepare prodrugs. As used herein, the term “prodrug” refers to any derivative of the compounds of general formula I, II, III, IV and V that are metabolized or otherwise converted into an active form upon introduction into the body of an individual or animal. Prodrugs are well known to those skilled in the art of pharmaceutical chemistry and provide benefits such as increased adsorption and half-life. Those skilled in the art of drug delivery will readily appreciate that the pharmacokinetic properties of general formulas I, II, III, IV and V may be controlled by an appropriate choice of moieties to produce prodrug derivatives.

One or more disclosed compounds, typically after purification, can be incorporated into a pharmaceutical composition or medicament. The disclosed compounds can be administered by a variety of known methods, including, for example, orally, rectally, or by parenteral routes (e.g., intramuscular, intravenous, subcutaneous, nasal or topical). The form in which the compounds are administered will be determined by the route of administration. Such forms include, but are not limited to, capsular and tablet formulations (for oral and rectal administration), liquid formulations (for oral, intravenous, intramuscular, subcutaneous, ocular, intranasal, inhalation-based and transdermal administration), powder formulations, and slow releasing microcarriers (for rectal, intramuscular or intravenous administration). The pharmaceutical composition or medicament can also contain a pharmaceutically acceptable vehicle, diluent, excipient or carrier and optional adjuvants, flavorings, colorants, wetting agents, emulsifying agents, pH buffering agents and preservatives. Some suitable pharmaceutically acceptable vehicles include, for example, saline, sterile water, Ringer's solution and isotonic sodium chloride solutions. The specific dosage level of active ingredient will depend upon a number of factors, including, for example, biological activity of the particular preparation, age, body weight, sex and general health of the individual being treated.

For topical use, creams, ointments, jellies, solutions or suspensions containing the compounds of the present disclosure are employed. Topical application is also meant to include the use of mouth washes and gargles.

Powder formulations may be delivered through mechanical power sprayers, nasal inhalers and nebulizers/atomizers. Prior to delivery, powder formulations may be solubilized in suitable solvents including water and saline solutions. In one aspect described herein the compound may be solubilized in a saline solution. In another aspect described herein a therapeutically effective amount of the compound delivered to the lungs.

Commonly used pharmaceutical ingredients which may be used as appropriate to formulate the composition for its intended route of administration include: acidifying agents, for example, but are not limited to, acetic acid, citric acid, fumaric acid, hydrochloric acid, nitric acid; and alkalinizing agents such as, but are not limited to, ammonia solution, ammonium carbonate, diethanolamine, monoethanolamine, potassium hydroxide, sodium borate, sodium carbonate, sodium hydroxide, triethanolamine, or trolamine.

Other pharmaceutical ingredients include, for example, but are not limited to, adsorbents (e.g., powdered cellulose and activated charcoal); aerosol propellants (e.g., carbon dioxide, CCI2F2, F2CIC—CCIF2 and CCIF3); air displacement agents (e.g., nitrogen and argon); antifungal preservatives (e.g., benzoic acid, butylparaben, ethylparaben, methylparaben, propylparaben, sodium benzoate); antimicrobial preservatives (e.g., benzalkonium chloride, benzethonium chloride, benzyl alcohol, cetylpyridinium chloride, chlorobutanol, phenol, phenylethyl alcohol, phenylmercuric nitrate and thimerosal); antioxidants (e.g., ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite); binding materials (e.g., block polymers, natural and synthetic rubber, polyacrylates, polyurethanes, silicones and styrene-butadiene copolymers); buffering agents (e.g., potassium metaphosphate, potassium phosphate monobasic, sodium acetate, sodium citrate anhydrous and sodium citrate dihydrate); carrying agents (e.g., acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection); chelating agents (e.g., edetate disodium and edetic acid); colorants (e.g., FD&C Red No. 3, FD&C Red No. 20, FD&C Yellow No. 6, FD&C Blue No. 2, D&C Green No. 5, D&C Orange No. 5, D&C Red No. 8, caramel and ferric oxide red); clarifying agents (e.g., bentonite); emulsifying agents (includes but are not limited to, acacia, cetomacrogol, cetyl alcohol, glyceryl monostearate, lecithin, sorbitan monooleate, polyethylene 50 stearate); encapsulating agents (e.g., gelatin and cellulose acetate phthalate); flavorants (e.g., anise oil, cinnamon oil, cocoa, menthol, orange oil, peppermint oil and vanillin); humectants (e.g., glycerin, propylene glycol and sorbitol); levigating agents (e.g., mineral oil and glycerin); oils (e.g., arachis oil, mineral oil, olive oil, peanut oil, sesame oil and vegetable oil); ointment bases (e.g., lanolin, hydrophilic ointment, polyethylene glycol ointment, petrolatum, hydrophilic petrolatum, white ointment, yellow ointment, and rose water ointment); penetration enhancers (transdermal delivery) (e.g., monohydroxy or polyhydroxy alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas); plasticizers (e.g., diethyl phthalate and glycerin); solvents (e.g., alcohol, corn oil, cottonseed oil, glycerin, isopropyl alcohol, mineral oil, oleic acid, peanut oil, purified water, water for injection, sterile water for injection and sterile water for irrigation); stiffening agents (e.g., cetyl alcohol, cetyl esters wax, microcrystalline wax, paraffin, stearyl alcohol, white wax and yellow wax); suppository bases (e.g., cocoa butter and polyethylene glycols (mixtures)); surfactants (e.g., benzalkonium chloride, nonoxynol 10, octoxynol 9, polysorbate 80, sodium lauryl sulfate and sorbitan monopalmitate); suspending agents (e.g., agar, bentonite, carbomers, carboxymethylcellulose sodium, hydroxyethyl cellulose, hydroxypropyl cellulose, hydroxypropyl methylcellulose, kaolin, methylcellulose, tragacanth and veegum); sweetening e.g., aspartame, dextrose, glycerin, mannitol, propylene glycol, saccharin sodium, sorbitol and sucrose); tablet anti-adherents (e.g., magnesium stearate and talc); tablet binders (e.g., acacia, alginic acid, carboxymethylcellulose sodium, compressible sugar, ethylcellulose, gelatin, liquid glucose, methylcellulose, povidone and pregelatinized starch); tablet and capsule diluents (e.g., dibasic calcium phosphate, kaolin, lactose, mannitol, microcrystalline cellulose, powdered cellulose, precipitated calcium carbonate, sodium carbonate, sodium phosphate, sorbitol and starch); tablet coating agents (e.g., liquid glucose, hydroxyethyl cellulose, hydroxypropyl cellulose, hydroxypropyl methylcellulose, methylcellulose, ethylcellulose, cellulose acetate phthalate and shellac); tablet direct compression excipients (e.g., dibasic calcium phosphate); tablet disintegrants (e.g., alginic acid, carboxymethylcellulose calcium, microcrystalline cellulose, polacrilin potassium, sodium alginate, sodium starch glycollate and starch); tablet glidants (e.g., colloidal silica, corn starch and talc); tablet lubricants (e.g., calcium stearate, magnesium stearate, mineral oil, stearic acid and zinc stearate); tablet/capsule opaquants (e.g., titanium dioxide); tablet polishing agents (e.g., carnuba wax and white wax); thickening agents (e.g., beeswax, cetyl alcohol and paraffin); tonicity agents (e.g., dextrose and sodium chloride); viscosity increasing agents (e.g., alginic acid, bentonite, carbomers, carboxymethylcellulose sodium, methylcellulose, povidone, sodium alginate and tragacanth); and wetting agents (e.g., heptadecaethylene oxycetanol, lecithins, polyethylene sorbitol monooleate, polyoxyethylene sorbitol monooleate, and polyoxyethylene stearate).

Some disclosed compounds were tested for CB1 and CB2 receptor binding affinity (Tables 1, 2, 3, 4, 5). As used herein “binding affinity” is represented by the Ki value. The Ki value is the affinity constant and describes the affinity of the compound for the receptor. The lower the Ki value, the higher the affinity of the compound for the receptor. A detailed description of the methods used to test “binding affinity” of compounds is given in Makriyannis, et al., US 2007/0135388 the content of which is hereby incorporated by reference.

The activity of some of the disclosed compounds for the CB1 and CB2 receptors was assessed by using the GTPγ-S binding assay (Tables 2,3,4). As used herein the “effect” of the compound on the GTPγ-S assay is represented by the EC₅₀ (GTP) value. The lower the EC₅₀ (GTP) value the higher the potency of the compound for the receptor A detailed description of the methods used to test compounds using the GTPγ-S assay is given in Parkkari, et al., Biorg. Med. Chem. Letters 2006, 16(9), 2437 and Griffin, et al., J. Pharmacol. Exp. Ther. 1998, 285(2), 553-560 the content of which is hereby incorporated by reference.

Further functional characterization of some of the disclosed compounds tested initially for “binding affinity” or for GTPγ-S binding was performed by using the cyclic adenosine monophosphate (cAMP) assay. Key compounds initially characterized functionally using the GTPγ-S assay were also doubly tested using the cAMP assay to unambiguously confirm their functional profiles. The functional potency of the compounds tested using the cyclase assay is also represented by the EC₅₀ (cAMP) value and similarly the lower the EC₅₀ (cAMP) value the higher the potency of the compound for the receptor. A detailed description of the methods used to test compounds using the cAMP assay is given in Yingpeng, et al. J. Med. Chem. (2018) 61, 8639-8657 the content of which is hereby incorporated by reference. The EC₅₀ values (potency of a test compound) of the functional assays may or may not be accompanied by the E₍ₘₐₓ₎ value in tables 1-5 (efficacy of the test compound). E₍ₘₐₓ₎/efficacy is the maximum GTP stimulation or cAMP inhibition levels and is presented as the percentage of maximum response evoked by a standard CB1/CB2 full agonist (e.g., CP-55,940) for reference see the paper cited above. If a test compound has an efficacy close to 100% of the standard full agonist, then the test compound is also classified as full agonist. If the efficacy of the test compound is lower than that of the standard, then the test compound is classified as partial agonist.

Unless otherwise specifically defined, “polarity” or “polar characteristics” of a compound refers to the calculated log P (c log P) and total polar surface area (tPSA) of each respective compound. These values have been calculated using the ChemBioDraw Ultra 14.0 software. The polar characteristics/polarity of a compound are important parameters that determine in vivo efficacy, potency, water solubility, tissue exposure and retention and in some cases toxicity. (See Nikas, et al. J. Med. Chem. (2015) 58, 609). Generally, the lower the c log P and the higher the tPSA values of a compound the higher the polarity/polar character of that given compound. This is a highly desirable property in the drug discovery process because more polar compounds are expected to exhibit enhanced druggability profiles including for instance better water solubility, absorption, oral bioavailability, excretion and detoxification.

In an aspect is a pharmaceutical composition comprising a therapeutically effective amount of a compound disclosed herein or a pharmaceutically acceptable salt thereof and a pharmaceutically acceptable carrier.

In another aspect, method of treating a condition, disorder, or disease in a subject in need thereof is disclosed, the method comprising administering to the subject a therapeutically effective amount of a compound or pharmaceutically acceptable salt or a pharmaceutical composition disclosed herein; wherein the condition, disorder, or disease is selected from the group consisting of pain; central pain; peripheral pain; neuropathic pain; neuropathy; inflammatory pain; neurodegenerative diseases including multiple sclerosis, Parkinson's disease, Huntington's chorea, Alzheimer's disease and amyotrophic lateral sclerosis; mental disorders such as schizophrenia and depression; mood disorders; addiction disorders; memory disorders; gastrointestinal motility disorders such as irritable bowel syndrome and diarrhea; dyskinesia; migraine; osteoporosis, osteoarthritis; high blood pressure disease or hypertension; peripheral vascular disease; coronary artery disease; abnormal heart rate; cardiac insufficiency; pulmonary hypertension; ocular hypertension or glaucoma; endotoxic shock; hypotensive shock; appetite disorders; immune system disorders; fertility disorders; diseases associated with motor dysfunction such as Tourette's syndrome; inflammation; neurological disorders; epilepsy; nausea; AIDS wasting syndrome; HIV and HIV related disorders; autism and autism spectrum disorder; cancer.

Testing of some compounds disclosed herein for their binding affinities and/or their functional properties for the CB1 and the CB2 cannabinoid receptors, showed very high affinity and/or very high potency for the two cannabinoid receptors (see Tables 1-5). Therefore, another aspect of the present disclosure is use of at least one of the disclosed compounds, and physiologically acceptable salts thereof, to bind to and to stimulate cannabinoid receptors.

In another aspect, a method of stimulating a cannabinoid receptor in a subject is disclosed, the method comprising: administering to the subject an effective amount of a compound or pharmaceutically acceptable salt or a pharmaceutical composition disclosed herein.

In another aspect, use of a compound or pharmaceutically acceptable salt or a pharmaceutical composition disclosed herein as: (a) a standard (either radioactive or not) for assay development, and/or (b) an imaging agent for imaging of cannabinoid receptors on cells, tissues, or organs, especially those carrying radioactive atoms. For example, following the same chemistry, compound 4.8.5 can be synthesized with radioactive tritium atoms instead of deuterium atoms (and could be a novel radioligand for developing bio-assays). Compound 4.7.4 can be synthesized carrying radioactive fluorine ¹⁸F (and could be useful as a radiotracer, for example, for positron emission tomography imaging).

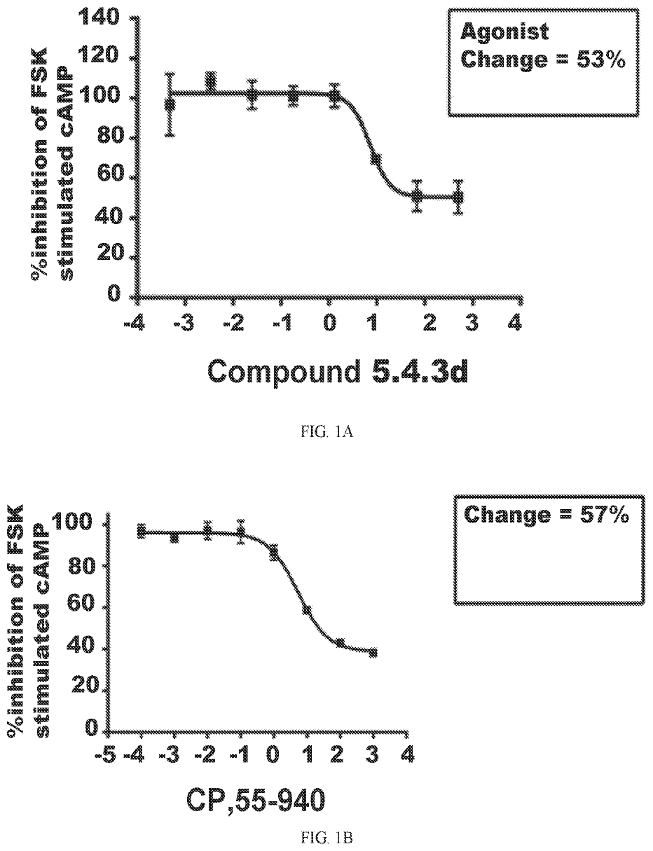

As concluded from the functional assays (e.g. cAMP) in some embodiments, the compound is a cannabinoid receptor full agonist. (e.g., Compound 5.4.3d, Table 3 and FIG. 1A and FIG. 1B)

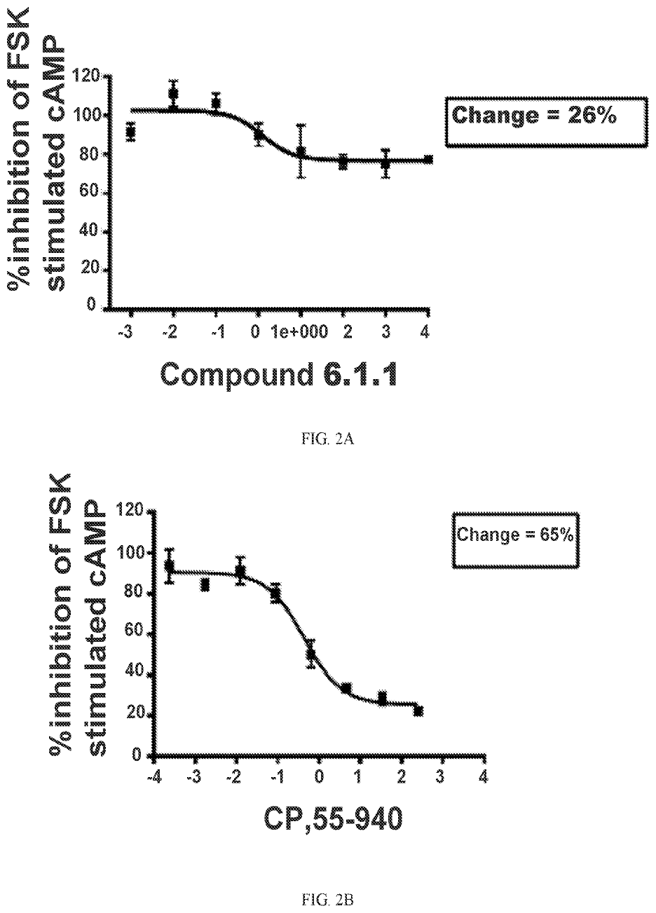

In some embodiments, the compound is a cannabinoid receptor partial agonist (e.g. Compound 6.1.1, Table 3 and FIG. 2A and FIG. 2B)

As seen in Tables 1-5 some of the invented compounds (e.g: nitrate ester ethanolamide analog 4.1.5b, table 1, and the phenyl AEA analog 4.3.5, table 5) bind to and stimulate preferentially the CB1 receptors. Meaning that these disclosed CB1 selective analogs are able to stimulate the CB1 receptor without affecting the CB2 receptor to the same degree. Therefore, in another aspect, a method of selectively stimulating a CB1 cannabinoid receptor in subject is disclosed, the method comprising: administering to the subject an effective amount of a compound or pharmaceutically acceptable salt or a pharmaceutical composition disclosed herein.

As seen in Table 6 where the polar characteristics of some compounds are provided, some of the invented compounds have very high polarity (e.g. omega substituted nitrate ester and cyano-analogs 4.1.5b, 4.1.5a in Tables 1 and 6). The high polarity of these ligands decreases their potential to cross the blood brain barrier and penetrate the CNS. Therefore, in another aspect, a method of selectively stimulating a cannabinoid receptor in the periphery of a subject is disclosed, the method comprising administering to the subject an effective amount of a compound or pharmaceutically acceptable salt or a pharmaceutical composition disclosed herein.

The disclosed compounds and pharmaceutically acceptable salts thereof, have high potential to be used as research tools to probe cannabinoid receptors and to uncover the biological roles of such receptors. For example, the disclosed compounds can be used as in vivo imaging agents; as molecular probes to help obtaining information about the exact binding site; as labels to screen for cells which express cannabinoid receptors (CB1 or CB2). The cannabinoid receptor ligands disclosed in this application can also be used as an aid in drug design, for example as new druggable leads, or as controls in assays for testing other compounds for their ability to bind to cannabinoid receptors. Other potential cannabinoid receptors that may be modulated by compounds of the present invention include GPR55, GPR18, and GPR119.

The disclosed compounds and physiologically acceptable salts thereof, when administered in therapeutically effective amounts, have high potential to bind to and modulate the CB1/CB2 cannabinoid receptors and thereby provide a physiological effect in an individual or animal that is useful to treat a condition in that individual or animal. Diseases, disorders, or conditions that may be treated by modulation of the CB1/CB2 cannabinoid receptors include for example: pain; central pain; peripheral pain; neuropathic pain; neuropathy; inflammatory pain; neurodegenerative diseases including multiple sclerosis, Parkinson's disease, Huntington's chorea, Alzheimer's disease and amyotrophic lateral sclerosis; mental disorders such as schizophrenia and depression; mood disorders; addiction disorders; memory disorders; gastrointestinal motility disorders such as irritable bowel syndrome and diarrhea; dyskinesia; migraine; osteoporosis, osteoarthritis; high blood pressure disease or hypertension; peripheral vascular disease; coronary artery disease; abnormal heart rate; cardiac insufficiency; pulmonary hypertension; ocular hypertension or glaucoma; to prevent or reduce endotoxic shock and hypotensive shock; to modulate appetite; to modulate the immune system; to modulate fertility; to prevent or reduce diseases associated with motor dysfunction such as Tourette's syndrome; to prevent or reduce inflammation; to provide neuroprotection; to produce peripheral vasodilation; to treat epilepsy; to treat nausea such as associated with cancer chemotherapy; AIDS wasting syndrome; HIV and HIV related disorders; autism and autism spectrum disorder; to treat several types of cancer as well as other ailments in which cannabinoid system is implicated.

In another aspect, the compounds of the present disclosure may be administered in combination with one or more additional therapeutic agent to improve the efficacy of other drugs. Potential other drugs include but not limited to: chemotherapeutic drugs including but not limited to camptothecin, indolizino, irinotecan, diflomotecan, exatecan, gimatecan, irinotecan, karenitecin, lurtorecan, rubitecan, silatecan, topotecan; NSAIDs including but not limited to salicylates, p-amino phenol derivatives, propionic acid derivatives, carboxylic acid derivatives, enolic acid derivatives, fenamic acid derivatives, sulphonanilides, and selective COX-2 inhibitors. “NSAID salicylates” include, but are not limited to, aspirin (acetylsalicylic acid), drfiunisal, and salsalate. “NSAID p-amino phenol derivatives” include, but are not limited to, paracetamol and phenacetin. “NSAID propionic acid derivatives” include, but are not limited to, ibuprofen, naproxen, fenoprofen, ketoprofen, dexketoprofen, fluribiprofen, oxaprozin, and loxoprofen. “NSAID carboxylic acid derivatives” include, but are not limited to, indomethacin, sulindac, etodolac, ketorolac, diclofenac. NSAID carboxylic acid derivatives include NSAID acetic acid derivatives. NSAID carboxylic acid derivatives are also referred to herein as “carboxylic acid NSAIDs.” “NSAID enolic acid derivatives” include, but are not limited to, piroxicam, meloxicam, tenoxicam, droxicam, lomoxicam, and isoxicam. “NSAID fenamic acid derivatives” include, but are not limited to, mefenamic acid, meclofenamic acid, flufenamic acid, and tolfenamic acid. “NSAID sulphonanilides” include, but are not limited to, nimesulide. “NSAID selective COX-2 inhibitors” include, but are not limited to, celecoxib, rofecoxib, valdecoxib, parecoxib, lumiracoxib, etoricoxib, and firocoxib; antibiotics including not limited to cephalosporins such as cefixime and cefpodoxime, clindamycin, penicillins, fluoroquinolones such as ciprofloxacin and levofloxacin; immunosuppressants and anti-rejection drugs including but not limited to tacrolimus and cyclosporine, mycophenolate mofetil, mycophenolate sodium, azathioprine, sirolimus and prednisone; other p-glucuronidase substrate drugs including but not limited to morphine, paracetamol, oxazepam, androsterone, carbamazepine, codeine, lamotrigine, lorazepam, temazepam, testosterone, and zidovudine; analgesics including but not limited to analgesics suitable for use in the pharmaceutical compositions described herein include, for example, opioids, natural opium alkaloids, morphine, opium, hydromorphone, nicomorphine, oxycodone, dihydrocodeine, diamorphine, tapentadol, papaveretum, codeine, phenylpiperidine derivatives, ketobemidone, pethidine, fentanyl, diphenylpropylamine derivatives, dextromoramide, piritramide, dextropropoxyphene, bezitramide, methadone, benzomorphan derivatives, pentazocine, phenazocine, oripavine derivatives, buprenorphine, morphinan derivatives, butorphanol, nalbuphine, tilidine, tramadol, dezocine, salicylic acid and derivatives, acetylsalicylic acid, aloxiprin, choline salicylate, sodium salicylate, salicylamide, salsalate, ethenzamide, morpholine salicylate, dipyrocetyl, benorilate, diflunisal, potassium salicylate, guacetisal, carbasalate calcium, imidazole salicylate, pyrazolones, phenazone, metamizole sodium, aminophenazone, propyphenazone, nifenazone, anilides, paracetamol, phenacetin, bucetin, propacetamol, other analgesics and antipyretics such as, for example, rimazolium, glafenine, floctafenine, viminol, nefopam, flupirtine, or ziconotide.

Depending on the individual medicaments utilized in a combination therapy for simultaneous administration, they may be formulated in combination (where a stable formulation may be prepared and where desired dosage regimes are compatible) or the medicaments may be formulated separately (for concomitant or separate administration through the same or alternative routes).

The therapeutically effective dosage of the compounds of this disclosure may readily be determined for treatment of each desired indication. The amount of the active ingredient (e.g., compounds) to be administered in the treatment of one of these conditions may vary widely according to such considerations as the particular compound and dosage unit employed, the mode of administration, the period of treatment, the age and sex of the patient treated, and the nature and extent of the condition treated.

The total amount of the active ingredient to be administered may generally range from about 0.0001 mg/kg to about 10 mg/kg, and preferably from about 0.001 mg/kg to about 10 mg/kg body weight per day. A unit dosage may contain from about 0.05 mg to about 500 mg of active ingredient, and may be administered one or more times per day. The daily dosage for administration by injection, including intravenous, intramuscular, subcutaneous, and parenteral injections, and use of infusion techniques may be from about 0.0001 mg/kg to about 10 mg/kg. The daily rectal dosage regimen may be from 0.0001 mg/kg to 10 mg/kg of total body weight. The transdermal concentration may be that required to maintain a daily dose of from 0.0001 mg/kg to 10 mg/kg. The daily inhaled concentration may be that required to maintain a daily dose of from 0.0001 mg/kg to 10 mg/kg.

Those skilled in the art will recognize, or be able to ascertain, using no more than routine experimentation, numerous equivalents to the specific substances and procedures described herein. Such equivalents are intended to be encompassed in the scope of the claims that follow the examples below.

### EXAMPLE ADVANTAGES

The disclosure provides novel design approaches for eCB lipids and presents the development of cannabinoid compounds with enhanced druggability profiles that resemble the structures of the endogenously produced cannabinoids AEA and 2-AG.

When compared to AEA or 2-AG, or currently existing eCB analogs, the compounds of the disclosure exhibit superior and/or unique pharmacodynamic and pharmacokinetic profiles that can be summarized as follows: a) higher binding affinities for the cannabinoid receptors CB1 and/or CB2; b) greater functional potencies for CB1 and/or CB2; c) diverse functional profiles (partial or full agonists at CB1 and CB2); d) enhanced rigidity of the flexible lipid chain imposed by novel design approaches that incorporate phenyl groups, cycloalkyl rings, chiral centers, and small polar groups at judiciously chosen positions within the arachidonoyl prototype or combinations thereof; e) enhanced stability to metabolizing enzymes; f) increased polarity; and g) improved chemical stability.

In some embodiments, advantages include increased safety and reduced abuse liability.

### EXAMPLE FEATURES

The present disclosure provides novel cannabinoid ligands that modulate cannabinoid receptors (for examples see Tables 1-5). The novel ligands disclosed here are termed “endocannabinoid analogs” (“eCB analogs”) because they possess structural similarities with the endogenously produced cannabinoids, or eCB lipids, namely anandamide (AEA) and 2-arachidonoyl glycerol (2-AG). AEA is characterized by an ethanolamide head group while 2-AG is characterized by a glyceride head. Despite possessing distinctly different head groups, AEA and 2-AG have three common pharmacophoric regions namely: a) a tetraolefinic chain (T.C., a moiety carrying four double bonds), b) a propyl linker that connects the tetraolefinic chain with the head group, and c) an n-pentyl tail that is connected to and terminates the tetraolefinic chain. Thus, the present disclosure encompasses two families of eCB analogs, AEA analogs, and 2-AG analogs.

Example unusual structural modifications of the invented AEA and 2-AG analogs over existing AEA and 2-AG compounds are: a) incorporation of a-electron rings (for example, phenyl) in the tetraolefinic chain of both AEA and 2-AG analogs; b) replacement of the linear propyl linker with cycloalkyl groups in both AEA and 2-AG analogs; c) addition of cycloalkyl groups at the allylic carbon where the tail connects with the tetraolefinic chain in both AEA and 2-AG analogs; d) addition of select, small, polar groups (e.g., cyano and nitrate ester) at the terminal carbon of the tail in both AEA and 2-AG analogs; e) incorporation of chiral centers in the structures of both AEA and 2-AG analogs (we have reported this modification for AEA analogs but not for 2-AG analogs); and f) hybrid modifications in both AEA and 2-AG analogs, meaning combinations of more than two modifications selected from a), b), c), d), and e) above.

These hybrid modifications can also combine one or more modification of the unusual a), b), c), d), or e) above, with an earlier reported modification at the head group (for example amide or ester) to provide structurally novel (unusual) AEA and 2-AG analogs.

It should be noted that AEA and 2-AG as well as other polyunsaturated fatty acid ethanolamides and glycerides are very sensitive to structural modifications, especially in the very flexible pharmacophores such as the tetraolefinic or polyunsaturated chain, and usually such modifications result in loss of biological activity. Surprisingly, many invented TC modified AEA and 2-AG analogs in this disclosure exhibit remarkably high affinities (K_{iS}) or functional potencies (EC₅₀s for the cannabinoid CB1 and CB2 receptors (see for example compounds in Tables 1, 2, 3, 4 and 5 and compare them with the endogenous AEA and 2-AG as well as with our earlier reported AEA analog AMG 315, that is currently the most potent AEA analog known) (see in another example in Table 7 the surprising 49-fold augmentation in the potency of AMG 315 for CB2 by changing the 20-CH₃ group to 20-CF₃ group seen in the hybrid analog 4.1.5c). It should be noted that currently no 2-AG analogs exist with potency similar to that of the endogenous 2-AG. Striking result is the exceptional potency of 2-AG analogs 5.1.6b, and 5.7.3b (Table 1, potency ~10-20 fold higher than 2-AG) which is comparable to the exogenous cannabinoid standard CP-55,940. In addition to enhancing affinity and in vitro potency of the AEA and 2-AG analogs our structural modifications confer to the molecules other desirable properties such as: a) increased polarity (drug-likeness property, see and compare analogs in Table 6); b) provide eCB analogs with variable functional profile such as partial agonists, e.g., 6.1.1. or full agonists, e.g., 5.4.3c, 5.4.3d, Table 3; and c) increase the enzymatic stability of the compounds.

Described herein is the successful incorporation of the flat and rigid phenyl ring in the tetraolefinic chain of the eCB template, chiral centers in the 2-AG and AEA templates, and conformationally constrained cycloalkyl rings as linker replacements in the endocannabinoid framework without compromising, or even enhancing, activity of the molecules for the cannabinoid receptors.

### EXAMPLE USES

The broad distribution of cannabinoid CB1 and CB2 receptors and their involvement in many biological and (patho)physiological processes makes them excellent targets for development of new therapeutics.

The design and synthesis of exogenous, THC-like, molecules to study cannabinoid receptors and develop THC-based drugs is well explored. However, there is only little progress towards developing endogenous-like molecules to probe the pharmacology of the endocannabinoid system and develop drugs based on the endocannabinoid chemical structure. The present disclosure provides endogenous-like cannabinoids that are useful as probe molecules to study the endocannabinoid system and to develop endocannabinoid-based therapeutics. Endocannabinoid based probes or drugs are expected to mimic the actions of the compounds produced in the body and therefore, they are expected not to exhibit the unwanted toxic side effects of the exogenous THC-like compounds. In summary, the endocannabinoid analogs disclosed herein are useful as safer probe molecules to explore cannabinoid pharmacology as well as drugs for an array of pathological and physiological conditions.

Pathological and physiological diseases, disorders, or conditions that may be treated by the modulation of CB1/CB2 cannabinoid receptors with endogenous AEA or 2-AG-like analogs include for example: diabetes, several types of cancer; neurodegenerative diseases including multiple sclerosis, Parkinson's disease, Huntington's chorea, Alzheimer's disease and amyotrophic lateral sclerosis; mental disorders such as schizophrenia and depression; mood disorders; addiction disorders; memory disorders; gastrointestinal motility disorders such as irritable bowel syndrome and diarrhea; dyskinesia; migraine; osteoporosis, osteoarthritis; high blood pressure disease or hypertension; peripheral vascular disease; coronary artery disease; abnormal heart rate; cardiac insufficiency; pulmonary hypertension; ocular hypertension or glaucoma; pain; central pain; peripheral pain; neuropathic pain; neuropathy; inflammatory pain; to prevent or reduce endotoxic shock and hypotensive shock; to modulate appetite; to modulate the immune system; to modulate fertility; to prevent or reduce diseases associated with motor dysfunction such as Tourette's syndrome; to image, prevent, or reduce inflammation; to provide neuroprotection; to produce peripheral vasodilation; to treat epilepsy; to treat nausea such as associated with cancer chemotherapy; AIDS wasting syndrome; HIV and HIV related disorders; autism and autism spectrum disorder, as well as other ailments in which the cannabinoid system is implicated.

In some embodiments, applications include therapeutics for pain, inflammation, glaucoma, cancer, and neurodegeneration.

In some embodiments, applications include 1) technologies for basic research on the endocannabinoid biochemical system in both academic and pharmaceutical institutions; and 2) high throughput and endogenous ligand-based assays for compounds targeting CB1 and CB2 cannabinoid receptors.

### B. EXAMPLES

It is understood that the skilled artisan may modify any of the examples, protocols and procedures in order to generate and/or test suitable compounds as described herein.

### Synthesis and Biological Testing of Compounds of General Formulas I, II, III, IV and V.

Synthesized compounds represented by the general structures I, II, III, IV and V are depicted in Tables 1, 2, 3, 4 and 5 respectively, on the following pages. Biological testing results and their comparison to the endogenous lipids 2-AG and AEA are also provided in Tables 1, 2, 3, 4 and 5. Table 6 depicts a comparison of the “polar characteristics”, (calculated c log P and total polar surface area) of some of the key disclosed compounds with either the endogenous lipid or previously reported endocannabinoid analogs. Syntheses of the corresponding carboxylic acid/ester precursors of these eCB analogs are depicted in Schemes 2, 4 and 5.

Receptor affinities are given as follows:
+: Ki=0.01-10 nM; ++: Ki=10-200 nM; +++: Ki=200-1,000 nM; ++++: Ki>1,000

TABLE 1
Compounds of the general formula I

| | | CB1 | | |
|---|---|---|---|---|
| | | receptor | | CB2 |
| | | affinity Ki | | receptor |
| | | (nM) | CB1 | Ki (nM) |
| | | and/or | Receptor Ki | and/or |
| Compound | | functional | with PMSF | functional |
| number | Structure | data | (nM) | data |
| AEA | 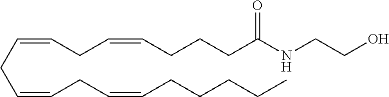 [SMILES: CCCCC/C=C\C/C=C\C/C=C\C/C=C\CCCC(=O)NCCO] | ++++ | ++ | ++ |
| 4.1.4 | 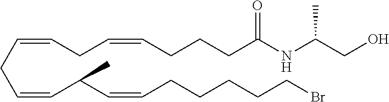 [SMILES: C[C@H](/C=C\C/C=C\C/C=C\CCCC(=O)N[C@H](C)CO)/C=C\CCCCCBr] | + | + | + |
| 4.1.5b | 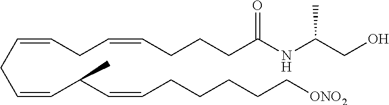 | + | + | ++ |
| 4.1.5a | 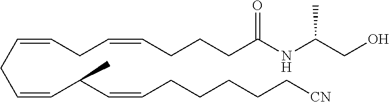 [SMILES: C[C@H](/C=C\C/C=C\C/C=C\CCCC(=O)N[C@H](C)CO)/C=C\CCCCCC#N] | Ki = + Functional cAMP data for CB1: EC₅₀ (cAMP) = 8.7 nM Eₘₐₓ = 76%, partial agonist | + | + |
| 4.1.5c | 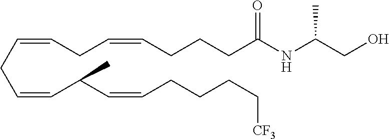 [SMILES: C[C@H](/C=C\C/C=C\C/C=C\CCCC(=O)N[C@H](C)CO)/C=C\CCCCC(F)(F)F] | − | − | − |
| 4.1.3 | 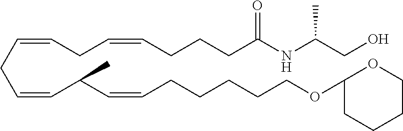 [SMILES: C[C@H](/C=C\C/C=C\C/C=C\CCCC(=O)N[C@H](C)CO)/C=C\CCCCCOC1CCCCO1] | ++++ | ++++ | ++++ |
| 4.7.2 | 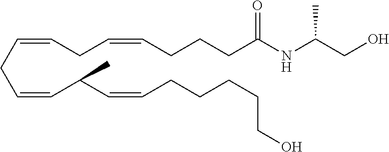 [SMILES: C[C@H](/C=C\C/C=C\C/C=C\CCCC(=O)N[C@H](C)CO)/C=C\CCCCCO] | ++ | − | ++ |
| 4.8.5 | 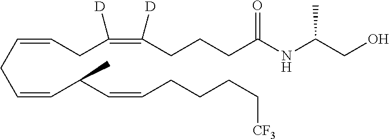 [SMILES: [2H]/C(C/C=C\C/C=C\[C@@H](C)/C=C\CCCCC(F)(F)F)=C(\[2H])CCCC(=O)N[C@H](C)CO] | − | − | − |
| 4.7.4 | 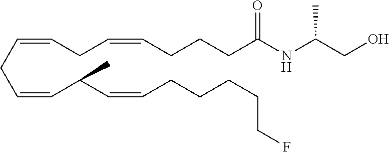 [SMILES: C[C@H](/C=C\C/C=C\C/C=C\CCCC(=O)N[C@H](C)CO)/C=C\CCCCCF] | + | − | ++ |
| 5.1.6b | 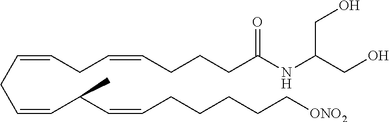 | EC₅₀ (cAMP) = + | − | EC₅₀ (cAMP) = ++ |
| 5.1.6a | 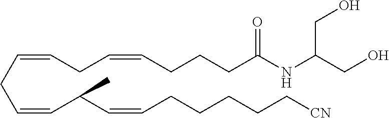 [SMILES: C[C@H](/C=C\C/C=C\C/C=C\CCCC(=O)NC(CO)CO)/C=C\CCCCCC#N] | EC₅₀ (cAMP) = ++ | − | EC₅₀ (cAMP) = ++ |
| 5.1.6c | 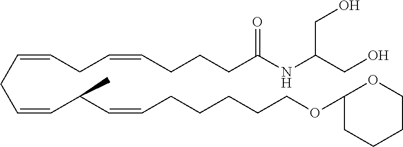 [SMILES: C[C@H](/C=C\C/C=C\C/C=C\CCCC(=O)NC(CO)CO)/C=C\CCCCCOC1CCCCO1] | EC₅₀ (cAMP) = ++++ | − | EC₅₀ (cAMP) = ++++ |
| 5.1.6d | 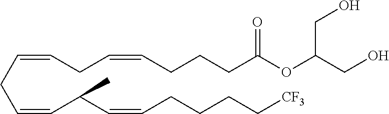 [SMILES: C[C@H](/C=C\C/C=C\C/C=C\CCCC(=O)OC(CO)CO)/C=C\CCCCC(F)(F)F] | − | − | − |
| 5.7.3b | 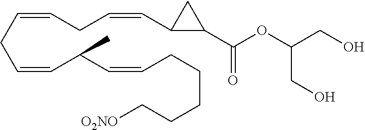 | EC₅₀ (cAMP) = + | − | − |
| 5.11.8 | 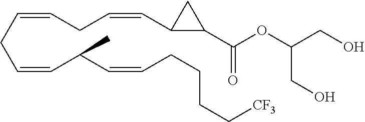 [SMILES: C[C@H](/C=C\C/C=C\C/C=C\C1CC1C(=O)OC(CO)CO)/C=C\CCCCC(F)(F)F] | EC₅₀ (cAMP) = + | − | EC₅₀ (cAMP) = ++ |
| 5.11.4 | 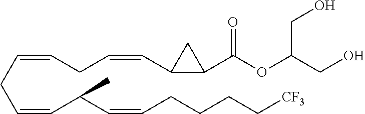 [SMILES: C[C@H](/C=C\C/C=C\C/C=C\C1CC1C(=O)OC(CO)CO)/C=C\CCCCC(F)(F)F] | EC₅₀ (cAMP) = ++ | − | EC₅₀ (cAMP) = ++ |
| 5.12.1 | 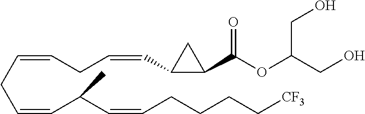 [SMILES: C[C@H](/C=C\C/C=C\C/C=C\[C@@H]1C[C@H]1C(=O)OC(CO)CO)/C=C\CCCCC(F)(F)F] | EC₅₀ (cAMP) = ++ | − | EC₅₀ (cAMP) = ++ |
| 5.12.2 | 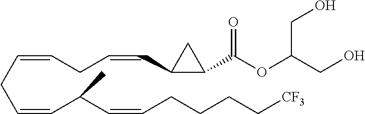 [SMILES: C[C@H](/C=C\C/C=C\C/C=C\[C@H]1C[C@@H]1C(=O)OC(CO)CO)/C=C\CCCCC(F)(F)F] | EC₅₀ (cAMP) = ++++ | − | EC₅₀ (cAMP) = ++ |
| 5.12.3 | 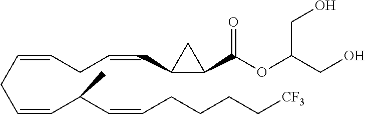 [SMILES: C[C@H](/C=C\C/C=C\C/C=C\[C@H]1C[C@H]1C(=O)OC(CO)CO)/C=C\CCCCC(F)(F)F] | EC₅₀ (cAMP) = + | − | EC₅₀ (cAMP) = ++ |
| 5.12.4 | 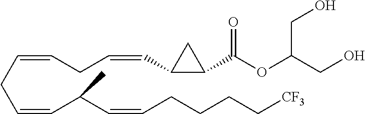 [SMILES: C[C@H](/C=C\C/C=C\C/C=C\[C@@H]1C[C@@H]1C(=O)OC(CO)CO)/C=C\CCCCC(F)(F)F] | EC₅₀ (cAMP) = ++ | − | EC₅₀ (cAMP) = ++ |

TABLE 2
Compounds of the general formula II

| Compound | | | |
|---|---|---|---|
| number | Structure | CB1 receptor | CB2 receptor |
| 2-AG | 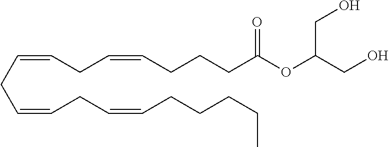 [SMILES: CCCCC/C=C\C/C=C\C/C=C\C/C=C\CCCC(=O)OC(CO)CO] | EC₅₀ (GTP) = +++ EC₅₀ (cAMP) = ++ | EC₅₀ (GTP) = +++ EC₅₀ (cAMP) = ++ |
| AEA | 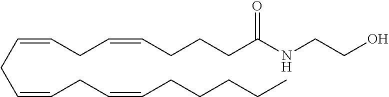 [SMILES: CCCCC/C=C\C/C=C\C/C=C\C/C=C\CCCC(=O)NCCO] | Ki (PMSF) = ++ | Ki = ++ |
| 5.2.2a | (trans) 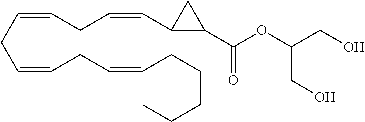 [SMILES: CCCCC/C=C\C/C=C\C/C=C\C/C=C\C1CC1C(=O)OC(CO)CO] | EC₅₀ (cAMP) = ++ | − |
| 4.2.2a | (trans) 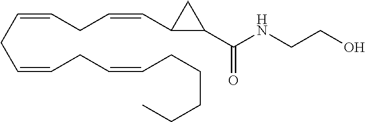 [SMILES: CCCCC/C=C\C/C=C\C/C=C\C/C=C\C1CC1C(=O)NCCO] | Ki = ++++ | Ki = ++++ |
| 5.2.2b | (cis) 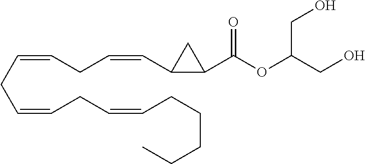 [SMILES: CCCCC/C=C\C/C=C\C/C=C\C/C=C\C1CC1C(=O)OC(CO)CO] | EC₅₀ (cAMP) = ++ | EC₅₀ (cAMP) = ++ |
| 4.2.2b | (cis) 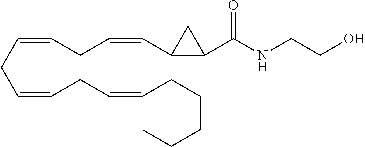 [SMILES: CCCCC/C=C\C/C=C\C/C=C\C/C=C\C1CC1C(=O)NCCO] | Ki = ++++ | Ki = ++++ |
| 5.2.2c | 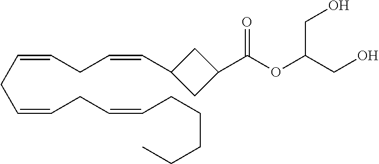 [SMILES: CCCCC/C=C\C/C=C\C/C=C\C/C=C\C1CC(C(=O)OC(CO)CO)C1] | EC₅₀ (cAMP) = ++++ | EC₅₀ (cAMP) = ++++ |
| 4.2.2c | 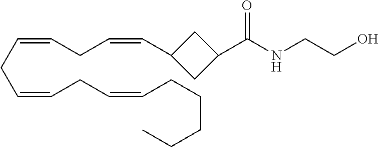 [SMILES: CCCCC/C=C\C/C=C\C/C=C\C/C=C\C1CC(C(=O)NCCO)C1] | Ki (PMSF) = +++ | Ki = +++ |
| 5.2.2d | 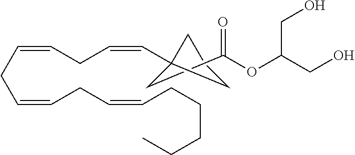 [SMILES: CCCCC/C=C\C/C=C\C/C=C\C/C=C\C1(CC(=O)OC(CO)CO)CCC1] | EC₅₀ (cAMP) = +++ | EC₅₀ (cAMP) = ++++ |
| 4.2.2d | 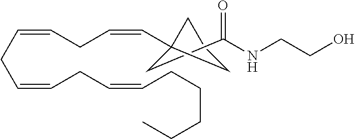 [SMILES: CCCCC/C=C\C/C=C\C/C=C\C/C=C\C1(CC(=O)NCCO)CCC1] | Ki (PMSF) = +++ | − |
| 5.2.2e | 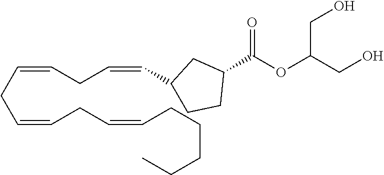 | EC₅₀ (GTP) = ++++ | EC₅₀ (cAMP) = +++ |
| 4.2.2e | 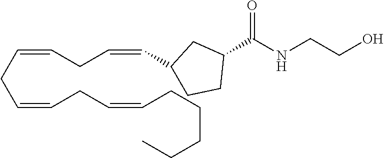 | Ki (PMSF) = +++ | Ki = +++ |
| 5.2.2f | 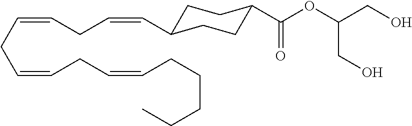 [SMILES: CCCCC/C=C\C/C=C\C/C=C\C/C=C\C1CCC(C(=O)OC(CO)CO)CC1] | EC₅₀ (cAMP) = ++++ | EC₅₀ (cAMP) = ++++ |
| 4.2.2f | 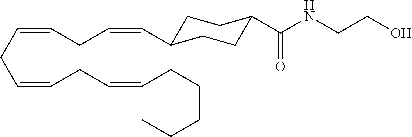 [SMILES: CCCCC/C=C\C/C=C\C/C=C\C/C=C\C1CCC(C(=O)NCCO)CC1] | Ki (PMSF) = +++ | Ki = +++ |
| 5.3.3 | 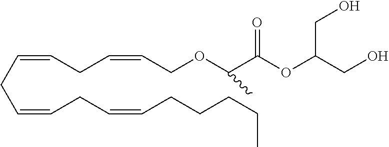 [SMILES: CCCCC/C=C\C/C=C\C/C=C\C/C=C\COC(C)C(=O)OC(CO)CO] | EC₅₀ (GTP) = ++++ | − |
| 5.8.3a | 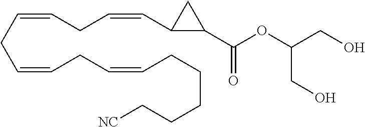 [SMILES: N#CCCCCC/C=C\C/C=C\C/C=C\C/C=C\C1CC1C(=O)OC(CO)CO] | EC₅₀ (cAMP) = ++ | − |
| 5.8.3b | 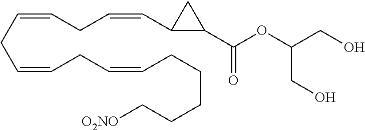 | EC₅₀ (cAMP) = ++ | − |

TABLE 3
Compounds of the general formula III

| Compound | | | |
|---|---|---|---|
| number | Structure | CB1 receptor | CB2 receptor |
| 2-AG | 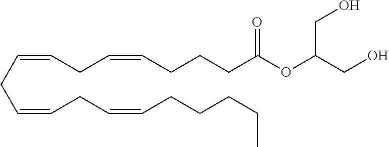 [SMILES: CCCCC/C=C\C/C=C\C/C=C\C/C=C\CCCC(=O)OC(CO)CO] | EC₅₀ (GTP) = +++ EC₅₀ (cAMP) = ++ | EC₅₀ (GTP) = +++ EC₅₀ (cAMP) = ++ |
| 5.4.3c | 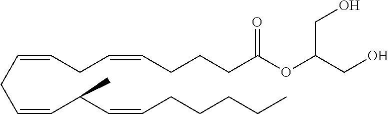 [SMILES: CCCCC/C=C\[C@H](C)/C=C\C/C=C\C/C=C\CCCC(=O)OC(CO)CO] | EC50 (cAMP) = 10.3 nM Eₘₐₓ = 99%, full agonist | EC50 (cAMP) = + |
| 5.4.3d | 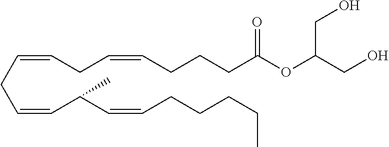 [SMILES: CCCCC/C=C\[C@@H](C)/C=C\C/C=C\C/C=C\CCCC(=O)OC(CO)CO] | EC₅₀ (cAMP) = 4.1 nM Eₘₐₓ = 95%, full agonist | EC50 (cAMP) = + |
| 6.2.2 | 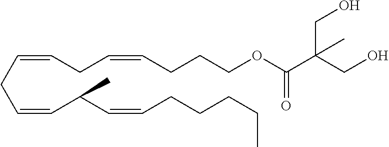 [SMILES: CCCCC/C=C\[C@H](C)/C=C\C/C=C\C/C=C\CCCOC(=O)C(C)(CO)CO] | EC₅₀ (cAMP) = ++ | EC₅₀ (cAMP) = ++ |
| 6.1.1 | 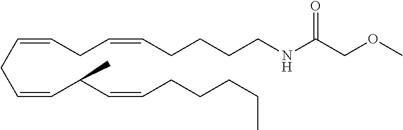 [SMILES: CCCCC/C=C\[C@H](C)/C=C\C/C=C\C/C=C\CCCCNC(=O)COC] | Ki = + | Ki = + EC₅₀ (cAMP) = 1.2 nM Eₘₐₓ = 40%, partial agonist |
| 5.4.3a | 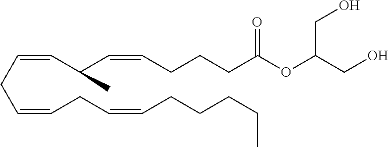 [SMILES: CCCCC/C=C\C/C=C\C/C=C\[C@H](C)/C=C\CCCC(=O)OC(CO)CO] | EC₅₀ (GTP) = ++++ | EC₅₀ (GTP) = +++ |
| 5.4.3b | 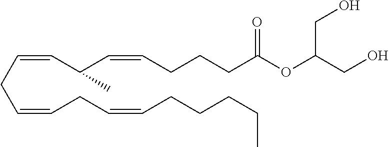 [SMILES: CCCCC/C=C\C/C=C\C/C=C\[C@@H](C)/C=C\CCCC(=O)OC(CO)CO] | EC₅₀ (GTP) = ++++ | EC₅₀ (GTP) = ++++ |
| 5.7.3a | 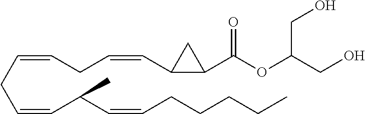 [SMILES: CCCCC/C=C\[C@H](C)/C=C\C/C=C\C/C=C\C1CC1C(=O)OC(CO)CO] | − | − |
| 5.7.3c | 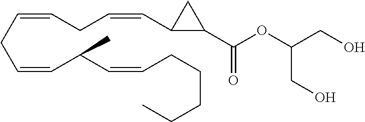 [SMILES: CCCCC/C=C\[C@H](C)/C=C\C/C=C\C/C=C\C1CC1C(=O)OC(CO)CO] | − | − |

TABLE 4
Compounds of the general formula IV

| Compound | | | |
|---|---|---|---|
| number | Structure | CB1 receptor | CB2 receptor |
| 2-AG | 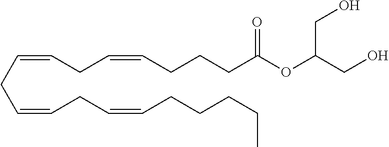 [SMILES: CCCCC/C=C\C/C=C\C/C=C\C/C=C\CCCC(=O)OC(CO)CO] | EC₅₀ (GTP) = +++ EC₅₀ (cAMP) = ++ | EC₅₀ (GTP) = +++ EC₅₀ (cAMP) = ++ |
| AEA | 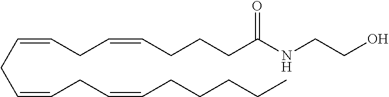 [SMILES: CCCCC/C=C\C/C=C\C/C=C\C/C=C\CCCC(=O)NCCO] | Ki (PMSF) = ++ | Ki = ++ |
| 4.4.3 | 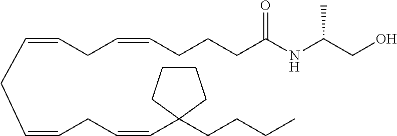 [SMILES: CCCCC1(/C=C\C/C=C\C/C=C\C/C=C\CCCC(=O)N[C@H](C)CO)CCCC1] | Ki (PMSF) = +++ | Ki (PMSF) = ++++ |
| 5.5.2 | 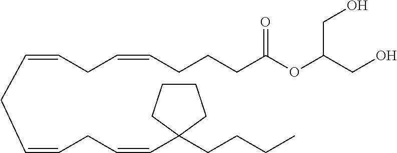 [SMILES: CCCCC1(/C=C\C/C=C\C/C=C\C/C=C\CCCC(=O)OC(CO)CO)CCCC1] | − | − |

TABLE 5
Compounds of the general formula V

| | | | CB1 | |
|---|---|---|---|---|
| | | | Receptor | CB2 |
| | | CB1 | Ki with | receptor |
| Compound | | receptor | PMSF | Ki |
| number | Structure | Ki (nM) | (nM) | (nM) |
| AEA | 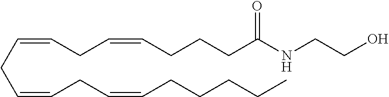 [SMILES: CCCCC/C=C\C/C=C\C/C=C\C/C=C\CCCC(=O)NCCO] | ++++ | ++ | ++ |
| 4.3.4 | 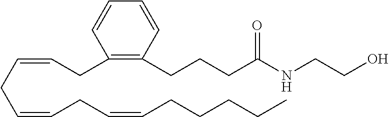 [SMILES: CCCCC/C=C\C/C=C\C/C=C\Cc1ccccc1CCCC(=O)NCCO] | ++ | ++ | ++ |
| 4.3.5 | 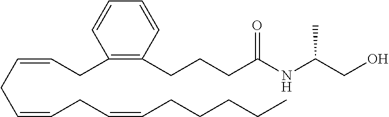 [SMILES: CCCCC/C=C\C/C=C\C/C=C\Cc1ccccc1CCCC(=O)N[C@H](C)CO] | + | ++ | ++ |
| 4.5.3 | 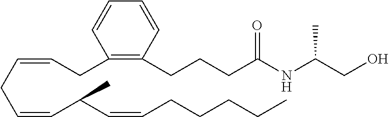 [SMILES: CCCCC/C=C\[C@H](C)/C=C\C/C=C\Cc1ccccc1CCCC(=O)N[C@H](C)CO] | + | + | − |
| 5.6.3 | 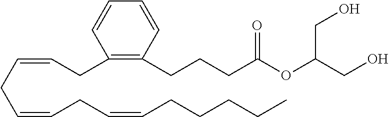 [SMILES: CCCCC/C=C\C/C=C\C/C=C\Cc1ccccc1CCCC(=O)OC(CO)CO] | − | − | − |
| 5.9.3 | 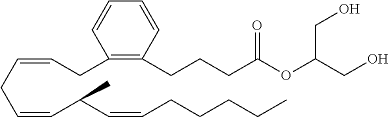 [SMILES: CCCCC/C=C\[C@H](C)/C=C\C/C=C\Cc1ccccc1CCCC(=O)OC(CO)CO] | EC₅₀ (cAMP) ++++ | − | EC₅₀ (cAMP) ++++ |
| 4.6.3 | 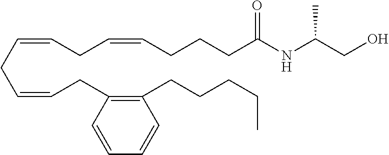 [SMILES: CCCCCc1ccccc1C/C=C\C/C=C\C/C=C\CCCC(=O)N[C@H](C)CO] | ++ | ++ | ++ |
| 5.10.3 | 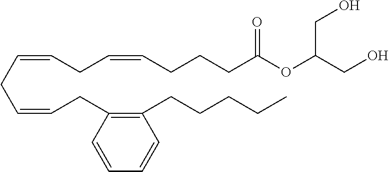 [SMILES: CCCCCc1ccccc1C/C=C\C/C=C\C/C=C\CCCC(=O)OC(CO)CO] | − | − | − |

TABLE 6
Comparison of polar characteristics

| Compound | | | |
|---|---|---|---|
| number | Structure | cLogP | tPSA |
| AEA | 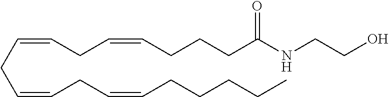 [SMILES: CCCCC/C=C\C/C=C\C/C=C\C/C=C\CCCC(=O)NCCO] | 6.1 | 49.33 |
| 2-AG | 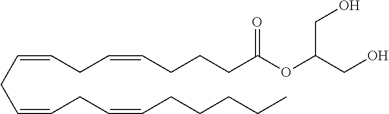 [SMILES: CCCCC/C=C\C/C=C\C/C=C\C/C=C\CCCC(=O)OC(CO)CO] | 6.8 | 66.76 |
| AMG315 Yingpeng, et al. J. Med. Chem. (2018) 61, 8639- 8657 | 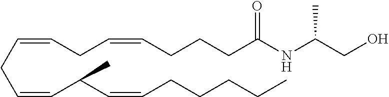 [SMILES: CCCCC/C=C\[C@H](C)/C=C\C/C=C\C/C=C\CCCC(=O)N[C@H](C)CO] | 6.8 | 49.33 |
| 4.1.5b | 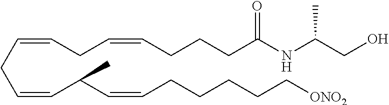 | 3.0 | 110.37 |
| 4.1.5a | 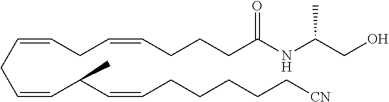 [SMILES: C[C@H](/C=C\C/C=C\C/C=C\CCCC(=O)N[C@H](C)CO)/C=C\CCCCCC#N] | 5.2 | 73.12 |

TABLE 7
Comparison of binding affinities for CB2 of the AEA analogs AM 356, AMG
315, and 4.1.5c. Compound 4.1.5c exhibits 49- and 61-fold higher binding affinity when
compared to AMG 315 and AM 356 respectively.

| | | hCB2 receptor |
|---|---|---|
| | | affinity Ki |
| Compound number | Structure | (nM) |
| AM 356 (one structural modification at C1′). Reported in Liu, Y., et al., J. Med. Chem., 2018, 61, 8639- 8657. | 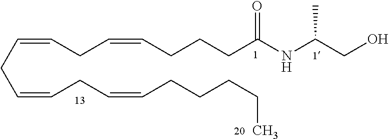 [SMILES: CCCCC/C=C\C/C=C\C/C=C\C/C=C\CCCC(=O)N[C@H](C)CO] | 220 |
| AMG 315 (two structural modifications at C1′ and C13). Reported in Liu, Y., et al., J. Med. Chem., 2018, 61, 8639- 8657. | 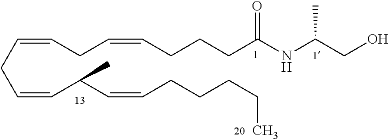 [SMILES: CCCCC/C=C\[C@H](C)/C=C\C/C=C\C/C=C\CCCC(=O)N[C@H](C)CO] | 176 |
| 4.1.5c (three structural modifications at C1′, C13, and C20). | 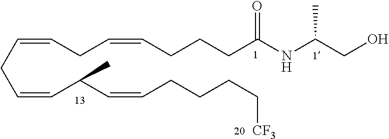 [SMILES: C[C@H](/C=C\C/C=C\C/C=C\CCCC(=O)N[C@H](C)CO)/C=C\CCCCC(F)(F)F] | 3.6 |

### Preparation of Compounds of Formulas I, II, III, IV and V

### 1. Synthesis of Chiral Synthons

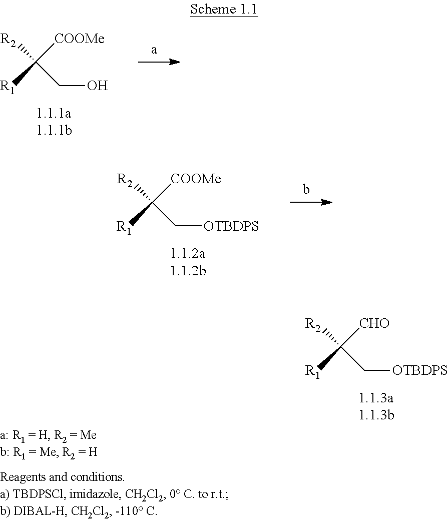

The experimental procedures as well as the detailed spectroscopic data are identical to those disclosed in Yingpeng, et al. J. Med. Chem. (2018) 61, 8639-8657, the content of which is hereby incorporated b reference.

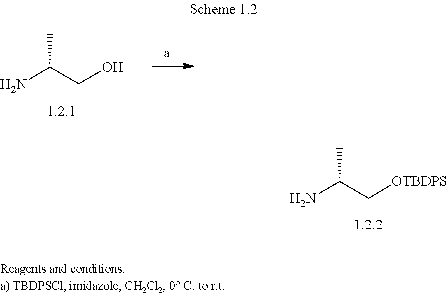

The experimental procedures as well as the detailed spectroscopic data are identical to those disclosed in Yingpeng, et al. J. Med. Chem. (2018) 61, 8639-8657, the content of which is hereby incorporated by reference.

### 2. Synthesis of Methyl- or Ethyl-Arachidonate-Like Precursors/Carboxylic Acids

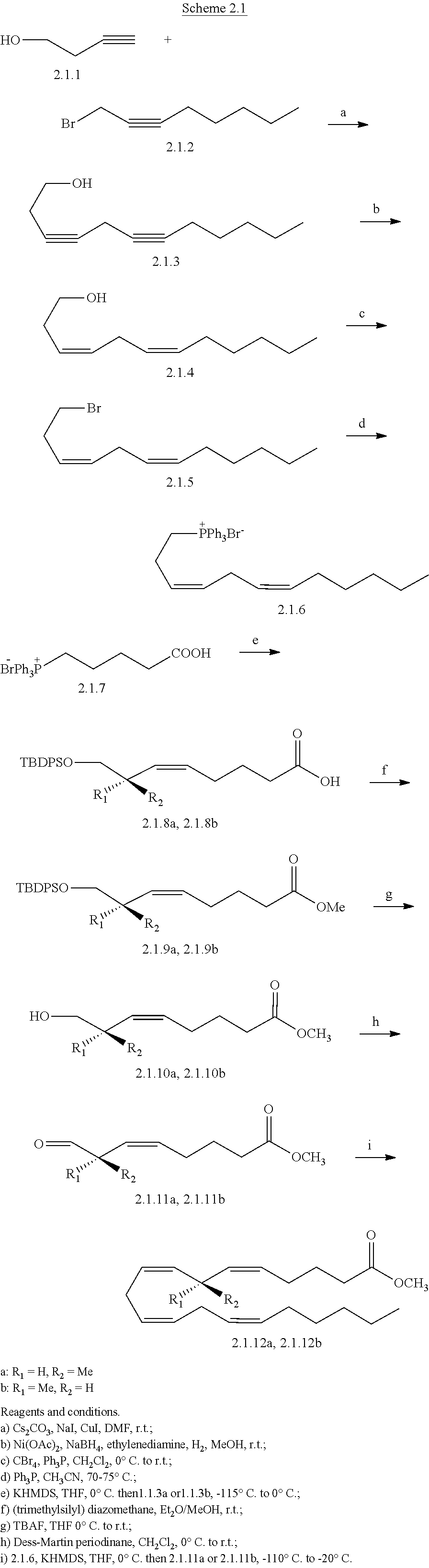

The experimental procedures as well as the detailed spectroscopic data are identical to those disclosed in Yingpeng, et al. J. Med. Chem. (2018) 61, 8639-8657, the content of which is hereby incorporated by reference.

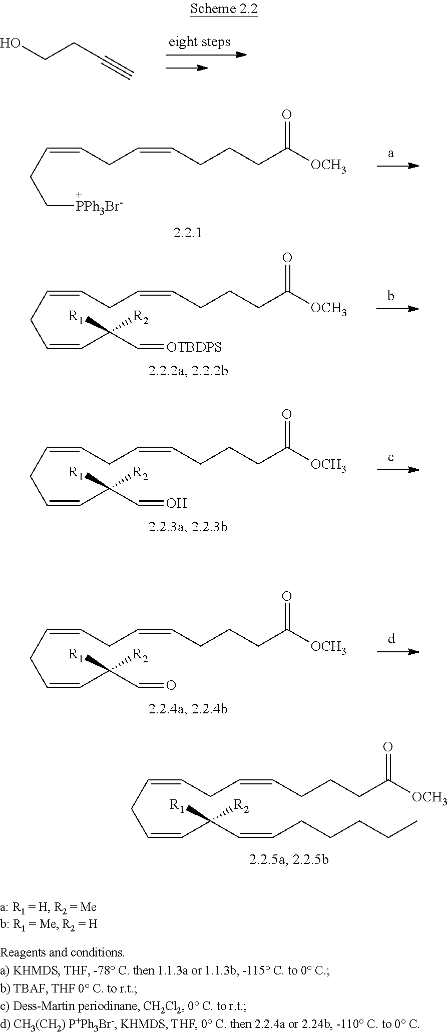

The experimental procedures as well as the detailed spectroscopic data are identical to those disclosed in Yingpeng, et al. J. Med. Chem. (2018) 61, the content of which is hereby incorporated by reference.

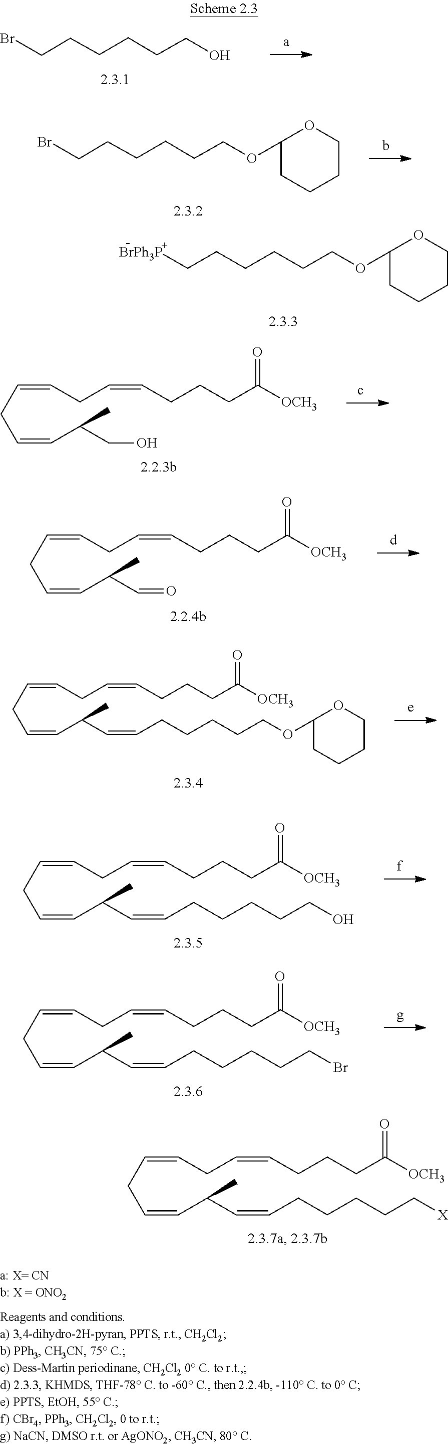

### General Procedure:

### 2-((6-Bromohexyl)oxy)tetrahydro-2H-pyran (compound 2.3.2)

To a stirred mixture of 6-bromohexan-1-ol (1 equiv.) and 3,4-dihydro-2H-pyran (1.5 equiv.) in dry dichloromethane (0.33 M) was added pyridinium p-toluenesulfonate (0.1 equiv.) and the mixture was stirred at room temperature, under an argon atmosphere for 4 hours. The reaction mixture was then diluted with dichloromethane and washed with water. The organic layer was separated, dried over MgSO₄ and the solvent was removed under reduced pressure. The residue was chromatographed on silica gel (10-20% diethyl ether-hexanes) to afford title compound in 78% yield as a colorless oil.

### Triphenyl(6-((tetrahydro-2H-pyran-2-yl)oxy)hexyl)phosphonium Bromide (Compound 2.3.3)

To a stirred solution of 2-((6-bromohexyl)oxy)tetrahydro-2H-pyran (1 equiv.) in dry acetonitrile (0.25 M) was added triphenylphosphine (2 equiv.) and the solution was stirred at 75° C., under argon, for 7-8 days. The solvent was evaporated under reduced pressure. The resulting residue was chromatographed on silica gel (7-20% methanol-dichloromethane) to afford title phosphonium salt in 85% yield as a yellow gum.

### Methyl (S,5Z,8Z,11Z)-13-methyl-14-oxotetradeca-5,8,11-trienoate (Compound 2.2.4b)

To a stirred solution of methyl (S,5Z,8Z,11Z)-14-hydroxy-13-methyltetradeca-5,8,11-trienoate (1 equiv.) in dichloromethane (0.05 M) at 0° C., under an argon atmosphere was added Dess-Martin periodinane (1.7 equiv.) The reaction mixture was stirred at 0° C. for 15 minutes and at room temperature for 3 hours. The reaction mixture was quenched by adding a mixture of Na₂S₂O₃ (10% in H₂O) and saturated Sodium bicarbonate (1:1) and diluted with Et₂O. The mixture was stirred vigorously and extracted with diethyl ether. The organic layer was washed with brine, dried over MgSO₄ and the solvent was evaporated under reduced pressure. The sensitive crude aldehyde was used for the next step immediately without further purification.

### Methyl (5Z,8Z,11Z,13S,14Z)-13-methyl-20-((tetrahydro-2H-pyran-2-yl)oxy)icosa-5,8,11,14-tetraenoate (Compound 2.3.4)

To a stirred solution of triphenyl(6-((tetrahydro-2H-pyran-2-yl)oxy)hexyl)phosphonium bromide (3 equiv.) in anhydrous THF (0.2 M) at −78° C. under an argon atmosphere was added potassium bis(trimethylsilyl)amide (2 equiv.) The mixture was stirred for 30 min to ensure complete formation of the orange ylide, and then it was cooled to −115° C. Subsequently, a solution of the above crude aldehyde (1 equiv.) in anhydrous THF was added dropwise. The reaction mixture was stirred for 25 minutes at −115° C., and then warmed to 0° C. over a 2 hour period. The reaction mixture was then cooled to −115° C. and quenched with a saturated aqueous sodium bicarbonate solution. The mixture was warmed to room temperature, extracted with Et₂O and the combined organic extracts were washed with brine, dried over MgSO₄ and concentrated under reduced pressure. The residue was chromatographed on silica gel (0-10% diethyl ether-hexanes) to afford title compound in 85% yield as a yellow oil. ¹H NMR (400 MHz, CDCl₃) δ 5.46-5.32 (m, 4H), 5.31-5.17 (m, 4H), 4.59 (dd, J=6.9, 3.8 Hz, 1H), 3.92-3.83 (m, 1H), 3.73 (dt, J=9.6, 6.9 Hz, 1H), 3.55-3.47 (m, 1H), 3.43 (ddq, J=15.9, 7.6, 7.3 Hz, 1H), 3.42-3.33 (m, 1H), 2.82 (dd, J=11.9, 5.9 Hz, 4H), 2.35 (t, J=7.5 Hz, 2H), 2.18-1.99 (m, 4H) [overlapping patterns i.e., 2.12 (t, J=7.5 Hz, 2H), 2.09-2.01 (m, 2H)], 1.82 (ddd, J=14.3, 10.0, 4.5 Hz, 1H), 1.76-1.65 (m, 3H)) [overlapping patterns i.e., 1.71 (quintet, J=7.5 Hz, 2H), 1.70 (quintet, J=7.5 Hz, 1H)], 1.64-1.56 (m, 4H), 1.57-1.47 (m, 4H), 1.41-1.32 (m, 4H), 1.01 (d, J=6.7 Hz, 3H).

### Methyl (S,5Z,8Z,11Z,14Z)-20-hydroxy-13-methylicosa-5,8,11,14-tetraenoate (Compound 2.3.5)

To a stirred solution of methyl (5Z,8Z,11Z,13S,14Z)-13-methyl-20-((tetrahydro-2H-pyran-2-yl)oxy)icosa-5,8,11,14-tetraenoate (1 equiv.) in ethanol (0.12 M) at room temperature under an argon atmosphere was added pyridinium p-toluenesulfonate (0.1 equiv.). The reaction mixture was stirred at 55° C. for 3 hours. The solvent was evaporated under reduced pressure and the resulting residue was chromatographed on silica gel (20-40% diethyl ether-hexanes) to afford title alcohol in 70% yield as a light yellow oil.

### Methyl (S,5Z,8Z,11Z,14Z)-20-bromo-13-methylicosa-5,8,11,14-tetraenoate (Compound 2.3.6)

To a stirred solution of methyl (S,5Z,8Z,11Z,14Z)-20-hydroxy-13-methylicosa-5,8,11,14-tetraenoate (1 equiv.) in dry dichloromethane (0.2 M) at −25° C. under an argon atmosphere, was added carbon tetrabromide (1.3 equiv.) and triphenylphosphine (1.3 equiv.). The reaction mixture was stirred at −25° C. for 1 hour and then at 0° C. for 30 minutes. The volatile materials were removed under reduced pressure and the resulting residue was chromatographed on silica gel (0-20% diethyl ether-hexanes) to afford title bromide in 97% yield as a light yellow oil. ¹³C NMR (100 MHz, CDCl₃) δ 174.00, 134.9, 134.7, 129.0, 128.9, 128.3, 128.2, 127.6, 125.7, 51.5, 33.8, 33.5, 32.7, 30.5, 28.9, 27.9, 27.3, 26.6, 25.8, 25.6, 24.8, 22.0.

### Methyl (S,5Z,8Z,11Z,14Z)-20-cyano-13-methylicosa-5,8,11,14-tetraenoate (Compound 2.3.7a)

To a stirred solution of methyl (S,5Z,8Z,11Z,14Z)-20-bromo-13-methylicosa-5,8,11,14-tetraenoate (1 equiv.) in anhydrous dimethyl sulfoxide (0.1 M) at room temperature, under an argon atmosphere was added sodium cyanide (10 equiv.) and the reaction mixture was stirred at ambient temperature overnight. The reaction mixture was quenched with ice-cold water and extracted with diethyl ether. The organic layer was washed with brine, dried over MgSO₄ and the solvent was evaporated under reduced pressure. The resulting crude residue was chromatographed on silica gel (20-30% diethyl ether-hexanes) to afford title nitrile in 94% yield as a colourless oil. ¹H NMR (500 MHz, CDCl₃) δ 5.49-5.35 (m, 3H), 5.31-5.17 (m, 4H), 3.66 (s, 3H), 3.53-3.39 (m, 1H), 2.89-2.73 (m, 4H), 2.37-2.27 (m, 4H) [overlapping patterns i.e., 2.34 (t, J=7.5 Hz, 2H), 2.29 (t, J=7.5 Hz, 2H)], 2.15-2.01 (m, 4H), 1.70 (quintet, J=7.5 Hz, 2H), 1.66 (quintet, J=7.3 Hz, 2H), 1.51-1.42 (m, 2H), 1.39 (dd, J=14.9, 7.3 Hz, 2H), 1.01 (d, J=6.8 Hz, 3H).

### Methyl (S,5Z,8Z,11Z,14Z)-13-methyl-20-(nitrooxy)icosa-5,8,11,14-tetraenoate (Compound 2.3.7b)

Ta a stirred solution of methyl (S,5Z,8Z,11Z,14Z)-20-bromo-13-methylicosa-5,8,11,14-tetraenoate (1 equiv.) in anhydrous acetonitrile (0.05 M) at room temperature, under an argon atmosphere was added silver nitrate (10 equiv.) and the reaction mixture was stirred at 80° C. overnight. The reaction mixture was diluted with diethyl ether and filtered through a pad of Celite. The filtrate was washed with ether and the solvent was evaporated under reduced pressure. The resulting crude residue was chromatographed on silica gel (0-15% diethyl ether-hexanes) to afford title nitrile in 78% yield as a light yellow oil. ¹H NMR (500 MHz, CDCl₃) δ 5.49-5.35 (m, 3H), 5.31-5.17 (m, 4H), 4.45 (t, J=7.5 Hz, 2H), 3.66 (s, 3H), 3.53-3.39 (m, 1H), 2.89-2.73 (m, 4H), 2.33 (t, J=7.5 Hz, 2H), 2.15-2.01 (m, 4H), 1.82-1.65 (m, 4H) [overlapping patterns i.e. 1.69 (quintet, J=7.3 Hz, 2H), 1.66 (quintet, J=7.5 Hz, 2H)], 1.47-1.34 (m, 4H), 1.01 (d, J=6.8 Hz, 3H).

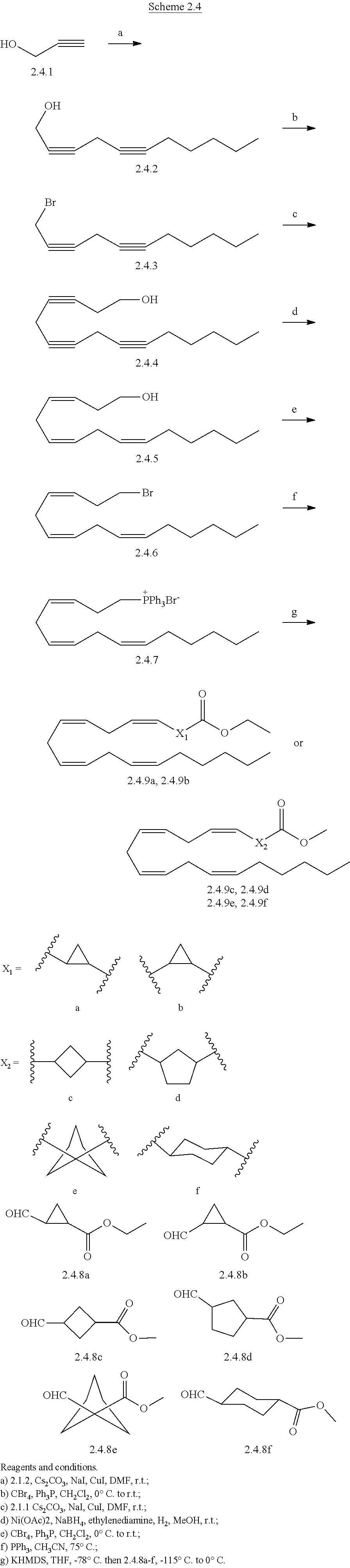

### Undeca-2,5-diyn-1-ol (Compound 2.4.2)

To a stirred mixture of Cs₂CO₃ (1 equiv.), NaI (1 equiv.) and CuI (1 equiv.) in anhydrous DMF [0.5 M] under an argon atmosphere at room temperature was added a solution of prop-2-yn-1-ol (1.2 equiv.) in anhydrous DMF. The suspension was stirred for 30 min. A solution of 1-bromooct-2-yne (1 equiv.) in anhydrous DMF was added and stirring was continued at room temperature for 2 h. The reaction mixture was then cooled to 0° C., diluted with Et₂O, quenched with dropwise addition of Sat. NH₄Cl and insoluble materials were dissolved by the addition of H₂O. The mixture was brought to room temperature and extracted with Et₂O. The combined organic extracts were washed with brine, dried over MgSO₄ and concentrated under reduced pressure. The resulting crude was chromatographed on silica gel (10-35% Et₂O/hexane) to afford pure title compound in 88% yield as light yellow oil. ¹H NMR (500 MHz, CDCl₃) δ 4.93 (br s, 1H), 4.27 (t, J=2.3 Hz, 2H), 3.19 (quintet, J=2.3 Hz, 2H), 2.15 (tt, J=7.2, J=2.3 Hz, 2H), 1.50 (quintet, J=7.2 Hz, 2H), 1.38-1.27 (m, 4H), 0.90 (t, J=7.1 Hz, 3H).

### 1-Bromoundeca-2,5-diyne (Compound 2.4.3)

Compound 2.4.3 was synthesized in a similar manner to compound 2.3.6. The volatile materials were removed under reduced pressure and the resulting residue was chromatographed on silica gel (0-10% Et₂O/hexanes) to afford title bromide in 91% yield as yellow oil. ¹H NMR (500 MHz, CDCl₃) δ 3.92 (t, J=2.3 Hz, 2H), 3.22 (quintet, J=2.3 Hz, 2H), 2.15 (tt, J=7.2, 2.3 Hz, 2H), 1.49 (quintet, J=7.2 Hz, 2H), 1.38-1.27 (m, 4H), 0.90 (t, J=7.1 Hz, 3H).

### Pentadeca-3,6,9-triyn-1-ol (Compound 2.4.4)

To a stirred mixture of Cs₂CO₃ (1 equiv.), NaI (1 equiv.) and CuI (1 equiv.) in anhydrous DMF [0.5 M] under an argon atmosphere at room temperature was added a solution of but-3-yn-1-ol (1.2 equiv.) in anhydrous DMF. The suspension was stirred for 30 min. A solution of 1-bromoundeca-2,5-diyne (1 equiv.) in anhydrous DMF was added and the reactions was kept at room temperature for 2 h. The reaction mixture was then cooled to 0° C., diluted with Et₂O, quenched by Sat. NH₄Cl and dissolved with the addition of a minimal amount of H₂O. The mixture was warmed to room temperature and extracted with Et₂O. The combined organic extracts were washed with brine, dried over MgSO₄ and concentrated under reduced pressure. The resulting crude was chromatographed on silica gel (10-35% Et₂O/hexane) to afford pure title compound in 88% yield as light yellow oil. ¹H NMR (500 MHz, CDCl₃) δ 5.02 (br s, 1H), 3.71 (t, J=6.2 Hz, 2H), 3.17-3.13 (m, 4H) [overlapping patterns i.e. 3.16 (quintet, J=2.3 Hz, 2H), 3.14 (quintet, J=2.3 Hz, 2H)], 2.45 (tt, J=6.2, J=2.3 Hz, 2H), 2.15 (tt, J=7.2, J=2.3 Hz, 2H), 1.49 (quintet, J=7.2 Hz, 2H), 1.38-1.27 (m, 4H), 0.90 (t, J=7.1 Hz, 3H).

### (3Z,6Z,9Z)-Pentadeca-3,6,9-trien-1-ol (Compound 2.4.5)

To a stirred mixture of Ni(OAc)₂ (1.7 equiv.) in anhydrous MeOH [0.06 M] under an argon atmosphere at 0° C. was added NaBH₄ (2 equiv.). The suspension was stirred for 15 min. Ethylenediamine (10 equiv.) was added and then the argon atmosphere was changed to H2 after 15 min. A solution of pentadeca-3,6,9-triyn-1-ol (1 equiv.) in anhydrous MeOH was added and the reactions was kept at room temperature for 1.5 h. The reaction suspension was filtrated through a pad of Celite and washed with Et₂O. The filtrate was concentrated under reduced pressure and extracted with Et₂O. The combined organic extracts were washed with brine, dried over MgSO₄ and concentrated under reduced pressure. The resulting crude was chromatographed on silica gel (5-20% Et₂O/hexane) to afford pure title compound in 96% yield as light yellow oil. ¹H NMR (500 MHz, CDCl₃) δ 5.59-5.53 (m, 1H), 5.45-5.30 (m, 5H), 3.67 (t, J=6.4 Hz, 2H), 3.49 (br s, 1H), 2.86 (tt, J=6.5 Hz, J=0.6 Hz, 2H), 2.82 (tt, J=6.5 Hz, J=0.6 Hz, 2H), 2.37 (qt, J=6.4 Hz, J=0.6 Hz, 2H), 2.05 (q, J=7.2 Hz, 2H), 1.41-1.24 (m, 6H), 0.89 (t, J=7.0 Hz, 3H).

### (3Z,6Z,9Z)-1-Bromopentadeca-3,6,9-triene (Compound 2.4.6)

Compound 2.4.6 was synthesized in a similar manner to compound 2.3.6. The volatile materials were removed under reduced pressure and the resulting residue was chromatographed on silica gel (0-10% Et₂O/hexane) to afford title bromide in 91% yield as yellow oil. ¹H NMR (500 MHz, CDCl₃) δ 5.56-5.50 (m, 1H), 5.44-5.30 (m, 5H), 3.38 (t, J=7.1 Hz, 2H), 2.86-2.79 (m, 4H) [overlapping patterns i.e. 2.83 (t, J=6.8 Hz, 2H), 2.81 (t, J=6.8 Hz, 2H)], 2.65 (qt, J=7.1 Hz, J=0.6 Hz, 2H), 2.05 (q, J=7.2 Hz, 2H), 1.41-1.24 (m, 6H), 0.89 (t, J=7.0 Hz, 3H).

### ((3Z,6Z,9Z)-Pentadeca-3,6,9-trien-1-yl(triphenylphosphonium Bromide (Compound 2.4.7)

Compound 2.4.7 was synthesized in a similar manner to compound 2.3.3. The resulting residue was chromatographed on silica gel (2-8% MeOH/CH₂Cl₂) to afford title phosphonium salt in 88% yield as yellow gum. ¹H NMR (500 MHz, CDCl₃) δ 7.89 (dd, J=12.6 Hz, J=0.8 Hz, 6H), 7.79 (td, J=7.7 Hz, J=1.5 Hz, 3H), 7.70 (td, J=7.7 Hz, J=3.3 Hz, 6H), 5.58-5.60 (m, 1H), 5.42-5.13 (m, 5H), 4.00 (dt, J=12.2 Hz, J=7.7 Hz, 2H), 2.63 (t, J=7.2 Hz, 2H), 2.56 (t, J=7.2 Hz, 2H)], 2.48 (dt, J=15.1 Hz, J=7.0 Hz, 2H), 1.98 (q, J=7.1 Hz, 2H), 1.37-1.20 (m, 6H), 0.89 (t, J=6.5 Hz, 3H).

### Ethyl 2-((1Z,4Z,7Z,10Z)-hexadeca-1,4,7,10-tetraen-1-yl)cyclopropane-1-carboxylate (Compound 2.4.9a)

To a stirred solution of ((3Z,6Z,9Z)-pentadeca-3,6,9-trien-1-yl)triphenylphosphonium bromide (3 equiv.) in anhydrous THF [0.17 M] at −78° C. under an argon atmosphere was added potassium bis(trimethylsilyl)amide (2 equiv.) The mixture was stirred for 30 min to ensure complete formation of the orange ylide, and then it was cooled to −115° C. Subsequently, a solution of ethyl 2-formylcyclopropane-1-carboxylate (1 equiv.) in anhydrous THF was added dropwise. The reaction mixture was stirred for 25 minutes at −115° C., and then warmed to 0° C. for 2 h. The reaction mixture was then cooled to −78° C., diluted with Et₂O and quenched with Sat. NaHCO₃. The mixture was brought to room temperature and extracted with Et₂O. The combined organic extracts were washed with brine, dried over MgSO₄ and concentrated under reduced pressure. The residue was chromatographed on silica gel (0-10% Et₂O/hexane) to afford title compound in 70% yield as light yellow oil. ¹H NMR (500 MHz, CDCl₃) δ 5.47-5.29 (m, 7H), 4.82 (tt, J=9.6 Hz, J=1.6 Hz, 1H), 4.14 (qd, J=7.1 Hz, J=0.6 Hz, 2H), 2.97-2.92 (m, 2H), 2.84 (t, J=5.2 Hz, 2H), 2.81 (t, J=6.3 Hz, 2H), 2.22-2.14 (m, 1H), 2.05 (q, J=6.9 Hz, 2H), 1.98 (quintet of d, J=4.0 Hz, J=1.0 Hz, 1H), 1.43-1.23 (m, 10H), 0.93-0.86 (m, 4H). Compounds 2.4.9b-f were synthesized in a similar manner.

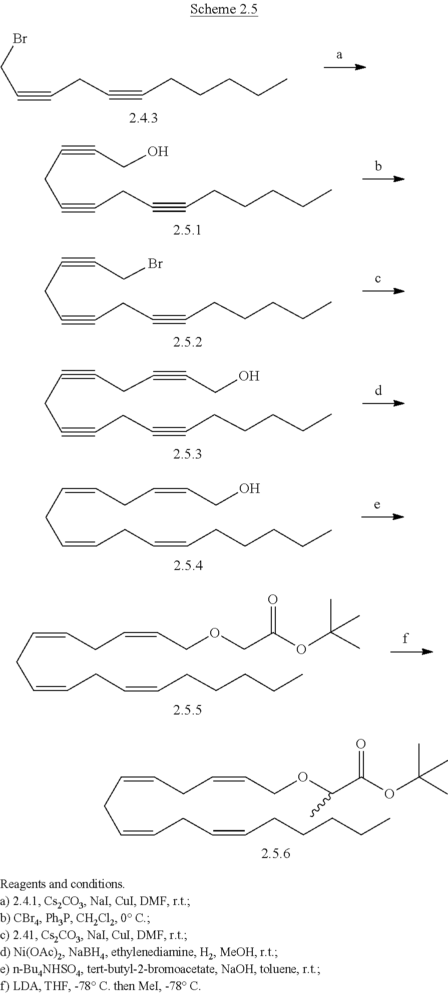

### Tetradeca-2,5,8-triyn-1-ol (Compound 2.5.1)

Compound 2.5.1 was synthesized in a similar manner to compound 2.4.2. The resulting crude product was chromatographed on silica gel (10-35% Et₂O/hexane) to afford pure title compound in 88% yield as light yellow oil. ¹H NMR (500 MHz, CDCl₃) δ 5.06 (br s, 1H), 4.26 (t, J=2.1 Hz, 2H), 3.21 (quintet, J=2.3 Hz, 2H), 3.14 (quintet, J=2.3 Hz, 2H), 2.15 (tt, J=7.2 Hz, J=2.3 Hz, 2H), 1.49 (quintet, J=7.2 Hz, 2H), 1.40-1.23 (m, 4H), 0.89 (t, J=7.1 Hz, 3H).

### 1-Bromotetradeca-2,5,8-triyne (Compound 2.5.2)

Compound 2.5.2 was synthesized in a similar manner to compound 2.3.6. The volatile materials were removed under reduced pressure and the resulting residue was chromatographed on silica gel (0-10% Et₂O/hexane) to afford title bromide in 91% yield as yellow oil. 1H NMR (500 MHz, CDCl₃) δ 5.06 (br s, 1H), 3.90 (t, J=2.3 Hz, 2H), 3.21 (quintet, J=2.3 Hz, 2H), 3.14 (quintet, J=2.3 Hz, 2H), 2.15 (tt, J=7.2 Hz, J=2.3 Hz, 2H), 1.49 (quintet, J=7.2 Hz, 2H), 1.40-1.23 (m, 4H), 0.89 (t, J=7.1 Hz, 3H).

### Heptadeca-2,5,8,11-tetrayn-1-ol (Compound 2.5.3)

Compound 2.5.3 was synthesized in a similar manner to compound 2.4.2. The resulting crude product was chromatographed on silica gel (10-35% Et₂O/hexane) to afford pure title compound in 88% yield as light yellow oil. ¹H NMR (500 MHz, CDCl₃) δ 5.04 (br s, 1H), 4.28-4.24 (m, 2H), 3.20 (quintet, J=2.2 Hz, 2H), 3.17-3.12 (m, 4H) [overlapping patterns i.e. 3.16 (quintet, J=2.2 Hz, 2H), 3.14 (quintet, J=2.2 Hz, 2H)], 2.15 (tt, J=7.2 Hz, J=2.3 Hz, 2H), 1.49 (quintet, J=7.2 Hz, 2H), 1.38-1.23 (m, 4H), 0.89 (t, J=7.1 Hz, 3H).

### (2Z,5Z,8Z,11Z)-Heptadeca-2,5,8,11-tetraen-1-ol (Compound 2.5.4)

Compound 2.5.4 was synthesized in a similar manner to compound 2.4.5. The resulting crude product was chromatographed on silica gel (5-20% Et₂O/hexane) to afford pure title compound in 96% yield as light yellow oil. ¹H NMR (500 MHz, CDCl₃) δ 5.67-5.59 (m, 1H), 5.57-5.50 (m, 1H), 5.44-5.29 (m, 6H), 4.23 (t, J=4.7 Hz, 2H), 4.20 (br s, 1H), 2.90-2.74 (m, 6H), 2.05 (dt, J=7.1 Hz, J=7.1 Hz, 2H), 1.40-1.23 (m, 6H), 0.89 (t, J=7.1 Hz, 3H).

### tert-Butyl 2-(((2Z,5Z,8Z,11Z)-heptadeca-2,5,8,11-tetraen-1-yl)oxy)acetate (Compound 2.5.5)

To a stirred mixture of compound 2.5.4 (1 equiv.) in toluene [0.5 M] under an argon atmosphere at room temperature was added n-Bu₄NHSO₄ (1.2 equiv.), tert-butyl-2-bromoacetate (6 equiv.) a sat. solution of NaOH (25 equiv.) in H₂O. The mixture was then stirred overnight. The reaction mixture was diluted with Et₂O, quenched by sat. NH₄Cl and extracted with Et₂O. The combined organic extracts were washed with brine, dried over MgSO₄, and concentrated under reduced pressure. The resulting crude residue was chromatographed on silica gel (5-20% Et₂O/hexane) to afford pure title compound in 60% yield as light yellow oil. ¹H NMR (500 MHz, CDCl₃) δ 5.65-5.55 (m, 2H), 5.44-5.29 (m, 6H), 4.19-4.16 (m, 2H), 3.95 (s, 2H), 2.90-2.74 (m, 6H), 2.05 (dt, J=7.0 Hz, J=7.0 Hz, 2H), 1.48 (s, 9H), 1.40-1.24 (m, 6H), 0.89 (t, J=7.1 Hz, 3H).

### tert-Butyl-2-(((2Z,5Z,8Z,11Z)-heptadeca-2,5,8,11-tetraen-1-yl)oxy)propanoate (Compound 2.5.6)

To a stirred mixture of compound 2.5.5 (1 equiv.) in anhydrous THF [0.5 M] under an argon atmosphere at −78° C. was added LDA dropwise. The reaction was left at −78° C. for 1 hour. MeI was added to the mixture and left at −78° C. for another 2 hours. The reaction mixture was brought to room temperature, diluted with Et₂O, quenched by sat. NH₄Cl and extracted with Et₂O. The combined organic extracts were washed with brine, dried over MgSO₄ and concentrated under reduced pressure. The resulting crude residue was chromatographed on silica gel (5-20% Et₂O/hexane) to afford pure title compound in 70% yield as light yellow oil. ¹H NMR (500 MHz, CDCl₃) δ 5.63-5.53 (m, 2H), 5.44-5.29 (m, 6H), 4.18 (dd, J=11.1 Hz, J=4.4 Hz, 1H), 4.04 (dd, J=11.1 Hz, J=4.4 Hz, 1H), 3.87 (q, J=6.8 Hz, 1H), 2.90-2.74 (m, 6H), 2.05 (dt, J=7.0 Hz, J=7.0 Hz, 2H), 1.48 (s, 9H), 1.40-1.24 (m, 9H) [overlapping patterns i.e. 1.37 (d, J=6.8 Hz, 3H), 1.40-1.24 (m, 6H)], 0.89 (t, J=7.1 Hz, 3H).

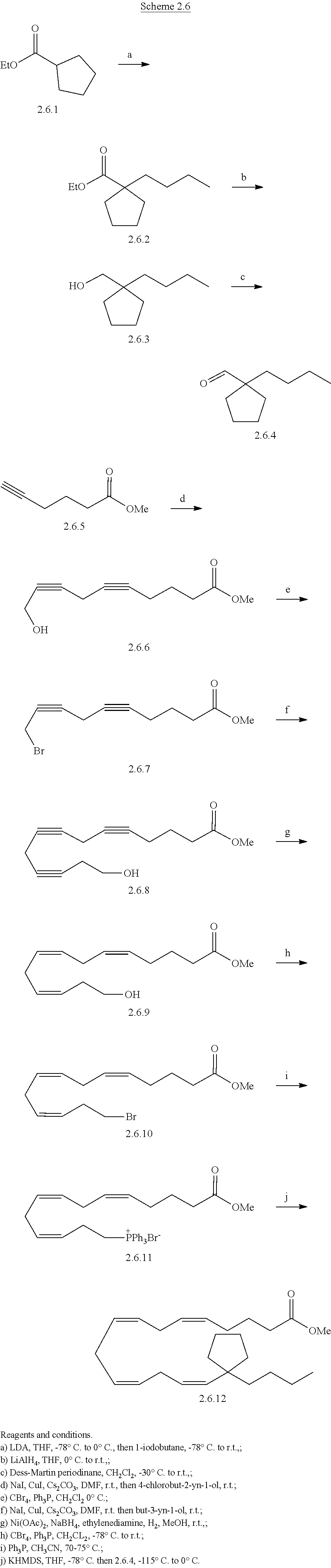

### 1-Butylcyclopentane-1-carbaldehyde (Compound 2.6.4)

The aldehyde 2.6.4 was synthesized in three steps from commercially available ethyl cyclopentanecarboxylate (2.6.1) by following a method described in Chisato Mukai et al. J. Am. Chem. Soc. (2012), 134, 19580-19583, the content of which is hereby incorporated by reference. The experimental procedures as well as the detailed spectroscopic data are identical to those disclosed in the above reference.

### Methyl (5Z,8Z,11Z)-14-hydroxytetradeca-5,8,11-trienoate (Compound 2.6.9)

The alcohol 2.6.9 was synthesized in four steps from commercially available methyl hex-5-ynoate (2.6.5) by following a method described in Fenmei Yao et al. Bioorg. Med. Chem. Lett. (2008), 18, 5912-5915, the content of which is hereby incorporated by reference. The experimental procedures as well as the detailed spectroscopic data are identical to those disclosed in the above reference.

### Methyl (5Z,8Z,11Z)-14-bromotetradeca-5,8,11-trienoate (Compound 2.6.10)

The bromide 2.6.10 was reported in Ming Yu et al. Bioorg. Med. Chem. (2003), 11, 2803-2821, of which the spectroscopic data are identical. However, in this synthesis the compound was synthesized by following our method described in Yingpeng, et al. J. Med. Chem. (2018) 61, 8639-8657, as shown below. To a stirred solution of 2.6.9 (1 equiv.) under an argon atmosphere at −78° C. in anhydrous dichloromethane (0.2 M) was added CBr₄ (3 equiv.) and PPh₃ (1 equiv.). The reaction mixture was left to room temperature and kept stirring overnight. Then the volatile materials were removed under reduced pressure. Purification by flash column chromatography on silica gel (0-20% diethyl ether in hexane) using the pre-absorption technique gave 2.6.10 in 98% yield, as a colorless oil.

### ((3Z,6Z,9Z)-14-Methoxy-14-oxotetradeca-3,6,9-trien-1-yl)triphenylphosphonium Bromide (Compound 2.6.11)

A stirred solution of 2.6.10 (1 equiv.) and dried triphenylphosphine (3 equiv.) in anhydrous CH₃CN (0.2 M) was heated (70-75° C.) for six days under an argon atmosphere. Solvent evaporation and purification by flash column chromatography on silica gel (5-15% MeOH/CH₂Cl₂) gave 2.6.11 in 87% yield as a brown gum. IR (neat): 3370, 3012, 2949, 2856, 1735 (C═O), 1651, 1436, 1153, 717, 667 cm¹; ¹H NMR (500 MHz, CDCl₃) δ 1.68 (quintet, J=7.5 Hz, 2H, 3-H), 2.05 (dt, J=7.2 Hz, J=7.2 Hz, 2H, 4-H), 2.30 (t, J=7.5 Hz, 2H, 1-H), 2.49 (m, 2H, 13-H), 2.57 (t, J=7.2 Hz, 2H), 2.63 (t, J=7.0 Hz, 2H), 3.65 (s, 3H, —COOCH₃), 4.01 (m, 2H, 14-H), 5.15-5.23 (m, 1H), 5.25-5.41 (m, 4H), 5.60-5.67 (m, 1H), 7.71 (m as td, J=7.4 Hz, J=3.4 Hz, 6H, 3-H, 5-H, —PPh₃), 7.80 (m as td, J=7.5 Hz, J=1.8 Hz, 3H, 4-H, —PPh₃), 7.90 (m as dd, J=12.5 Hz, J=7.5 Hz, 6H, 2-H, 6-H, —PPh₃). ¹³C NMR (100 MHz, CDCl₃) δ 173.9, 134.9, 134.9, 133.8, 133.7, 130.5, 130.4, 130.1, 129.1, 128.6, 128.5, 127.3, 126.8, 126.65, 118.8, 117.9, 53.4, 51.5, 33.3, 26.5, 25.5, 24.7, 23.2, 22.7, 20.5, 20.4.

### Methyl (5Z,8Z,11Z,14Z)-15-(1-butylcyclopentyl)pentadeca-5,8,11,14-tetraenoate (Compound 2.6.12)

To a solution of ((3Z,6Z,9Z)-14-Methoxy-14-oxotetradeca-3,6,9-trien-1-yl)triphenylphosphonium bromide (2.6.11) (1.2 equiv.) in anhydrous THF (0.15 M) at −78° C. under an argon atmosphere was added potassium bis(trimethylsilyl)amide 1M in THF (1.1 equiv.) dropwise. The mixture was stirred for 40 min to ensure complete formation of the orange ylide, and then it was cooled to −115° C. Subsequently, a solution of aldehyde 2.6.4 (1 equiv.) in anhydrous THF (1 mL) was added dropwise. The reaction mixture was stirred for 5 min at −115° C., and then warmed to 0° C. over a 3-hour period. The reaction mixture was then cooled to −115° C. and quenched with a saturated aqueous sodium bicarbonate solution. The mixture was warmed to room temperature, extracted with Et₂O and the combined organic extracts were washed with brine, dried over MgSO₄ and concentrated under reduced pressure. Purification by flash column chromatography on silica gel (3-10% Et₂O/hexane) gave 2.6.12 in 59% yield as a colorless oil. IR (neat): 3013, 2930, 2868, 1739 (C═O), 1462, 1430, 1106, 749, 721 cm⁻¹; ¹H NMR (500 MHz, CDCl₃) δ 5.44-5.31 (m, 7H), 5.19 (dt, J=11.6 Hz, J=7.5 Hz, 1H), 3.67 (s, 3H, —COOCH₃), 2.93-2.87 (m as t, J=7.4 Hz, 2H), 2.82 (dt, J=15.1 Hz, J=5.7 Hz, 4H), 2.32 (t, J=7.5 Hz, 2H, 2-H), 2.11 (dd, J=7.3 Hz, J=7.3 Hz, 2H, 4-H), 1.71 (quintet, J=7.4 Hz, 2H, 3-H), 1.69-1.64 (m, 2H), 1.63-1.51 (m, 6H), 1.48-1.40 (m, 2H), 1.29-1.21 (m, 4H), 0.88 (t, J=7.0 Hz, 3H, 20-H). ¹³C NMR (100 MHz, CDCl₃) δ 174.0, 139.2, 128.9, 128.8, 128.7, 128.1, 127.8, 127.5, 51.4, 47.9, 40.7, 39.6, 33.4, 28.0, 27.2, 26.5, 25.7, 25.6, 24.7, 24.1, 23.5, 14.1. LC/MS analysis (Waters MicroMass ZQ system) showed retention time 6.4 min for the title compound.

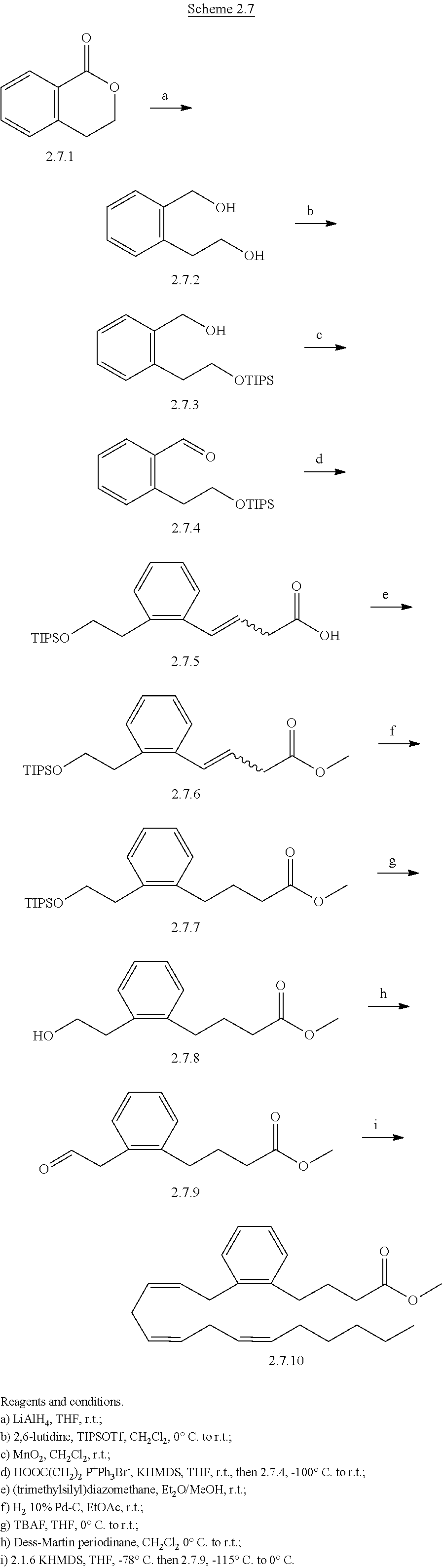

### 2-(2-(Hydroxymethyl)phenyl)ethan-1-ol (Compound 2.7.2)

To a stirred mixture of compound 2.7.1 (1 equiv.) in anhydrous THF [0.1 M] under an argon atmosphere at room temperature was added LiAlH₄ (2 equiv.). The reaction was stirred vigorously at room temperature overnight. The reaction mixture was cooled to 0° C. EtOAc and then 5% HCl in H₂O was added dropwise to the suspension. The mixture was then extracted with EtOAc. The combined organic extracts were washed with brine, dried over MgSO₄ and concentrated under reduced pressure. The resulting crude residue was chromatographed on silica gel (20-80% EtOAc/hexane) to afford pure title compound in 97% yield as light yellow oil. ¹H NMR (400 MHz, CDCl₃) δ 7.32-7.25 (m, 2H), 7.24-7.18 (m, 2H), 4.58 (s, 2H), 3.81 (t, J=5.8 Hz, 2H), 3.54 (br s, 1H), 2.89 (t, J=5.8 Hz, 2H). ¹³C NMR (100 MHz, CDCl₃) δ 139.24, 138.19, 130.05, 129.76, 128.54, 126.73, 63.32, 63.07, 35.03.

### (2-(2-((Triisopropylsilyl)oxy)ethyl)phenyl)methanol (Compound 2.7.3)

To a stirred mixture of compound 2.7.2 (1 equiv.) and 2,6-lutidine (2.2 equiv.) in anhydrous CH₂Cl₂ [0.6 M] under an argon atmosphere at 0° C. was added triisopropylsilyl trifluoromethanesulfonate (1.1 equiv.) dropwise. The mixture was then diluted with EtOAc, quenched with Sat. NH₄Cl, extracted with EtOAc. The combined organic extracts were washed with brine, dried over MgSO₄ and concentrated under reduced pressure. The resulting crude residue was chromatographed on silica gel (20-80% EtOAc/hexane) to afford pure title compound in 35% yield as light yellow oil. ¹H NMR (400 MHz, CDCl₃) δ 7.33-7.16 (m, 4H), 4.66 (d, J=5.6 Hz, 2H), 3.97 (t, J=5.9 Hz, 2H), 3.21 (t, J=5.6 Hz, 1H), 2.96 (t, J=5.9 Hz, 2H), 1.12-0.93 (m, 21H). ¹³C NMR (100 MHz, CDCl₃) δ 139.73, 138.22, 129.82, 129.32, 128.23, 126.55, 64.99, 63.53, 35.17, 17.72, 11.82.

### 2-(2-((Triisopropylsilyl)oxy)ethyl)benzaldehyde (Compound 2.7.4)

To a solution of compound 2.7.3 (1 equiv.) in dry CH₂Cl₂ [0.1 M] under an argon atmosphere at room temperature was added MnO₂ (10 equiv.). The reaction was stirred vigorously at room temperature overnight. The reaction mixture was filtered through filter paper and concentrated under reduced pressure. The resulting crude residue was chromatographed on silica gel (20-70% EtOAc/hexane) to afford pure title compound in 95% yield as light yellow oil. ¹H NMR (400 MHz, CDCl₃) δ 10.31 (s, 1H), 7.84 (dd, J=7.6 Hz, J=1.3 Hz, 1H), 7.50 (td, J=7.4 Hz, J=1.3 Hz, 1H), 7.40-7.30 (m, 2H), 3.93 (t, J=6.4 Hz, 2H), 3.27 (t, J=6.4 Hz, 2H), 1.12-0.90 (m, 21H). ¹³C NMR (100 MHz, CDCl₃) δ 192.47, 142.21, 134.48, 133.49, 132.03, 130.95, 126.75, 64.32, 35.66, 17.85, 11.86.

### 4-(2-(2-((Triisopropylsilyloxy)ethyl)phenylbut-3-enoic Acid (Compound 2.7.5)

To a solution of HOOC(CH₂)₂P⁺Ph₃Br⁻ (1.5 equiv.) in anhydrous THF under an argon atmosphere at room temperature was added a 1M solution of KHMDS (2.7 equiv.) in anhydrous THF dropwise. The reaction was left at room temperature for 1 hour. The reaction was cooled to −100° C. and a solution of compound 2.7.4 (1 equiv.) in anhydrous THF was added. The reaction was brought to room temperature over a period of 3.5 hours. The mixture was then diluted with EtOAc, quenched with Sat. NaHCO₃, extracted with EtOAc. The combined organic extracts were washed with brine, dried over MgSO₄ and concentrated under reduced pressure. The resulting crude was chromatographed on silica gel (20-80% EtOAc/hexane) to afford pure title compound in 84% yield as light yellow oil. ¹³C NMR (100 MHz, CDCl₃) δ 177.54, 137.38, 135.41, 131.96, 130.29, 129.08, 127.48, 126.11, 123.11, 63.75, 37.11, 33.61, 17.91, 11.92.

### Methyl 4-(2-(2-((triisopropylsilyl)oxy)ethyl)phenyl)but-3-enoate (Compound 2.7.6)

To a solution of compound 2.7.5 (1 equiv.) in a 2:1 mixture of Et₂O and MeOH under an argon atmosphere at room temperature was added (trimethylsilyl)diazomethane (3 equiv.). The reaction was left at room temperature for 1 hour. The mixture was concentrated under reduced pressure. The resulting crude residue was chromatographed on silica gel (10-40% EtOAc/hexane) to afford title compounds (cis and trans isomers) in 82% yield as light yellow oil. ¹H NMR (400 MHz, CDCl₃) δ 7.47-7.43 (m, 1H), 7.25-7.12 (m, 7H) [overlapping of cis and trans isomers], 6.83-6.75 (m, 2H), 6.17 (dt, J=12.5 Hz, J=5.7 Hz, 1H), 5.92 (dt, J=9.0 Hz, J=5.9 Hz, 1H), 3.83-3.76 (m, 4H) [overlapping of cis and trans isomers], 3.72 (s, 3H), 3.69 (s, 3H), 3.27 (dd, J=5.7 Hz, J=1.2 Hz, 2H), 3.17 (dd, J=5.9 Hz, J=1.4 Hz, 2H), 2.93 (t, J=6.0 Hz, 2H), 2.85 (t, J=5.8 Hz, 2H), 1.11-0.98 (m, 42H) [overlapping of cis and trans isomers].

### Methyl 4-(2-(2-((triisopropylsilyl)oxy)ethyl)phenyl)butanoate (Compound 2.7.7)

To a solution of compound 2.7.6 (1 equiv.) in anhydrous EtOAc under H2 at room temperature was added 10% Pd/C (17% w/w). The reaction was left for 1 hour. The reaction mixture was filtered through a pad of Celite, washed with CH₂Cl₂. The filtrate was concentrated under reduced pressure. The resulting crude residue was chromatographed on silica gel (10-40% EtOAc/hexane) to afford pure title compounds in 99% yield as light yellow oil. ¹³C NMR (100 MHz, CDCl₃) δ 173.80, 139.68, 136.74, 130.17, 129.20, 126.34, 126.09, 64.47, 51.47, 36.19, 33.72, 32.05, 26.30, 17.94, 11.94.

### Methyl 4-(2-(2-hydroxyethyl)phenyl)butanoate (Compound 2.7.8)

Compound 2.7.8 was synthesized in a similar manner to compound 2.1.10a. The resulting crude product was chromatographed on silica gel (10-40% EtOAc/hexane)) to afford pure title compound in 99% yield as light yellow oil. ¹³C NMR (100 MHz, CDCl₃) δ 173.89, 139.91, 136.10, 129.90, 129.57, 126.66, 126.35, 63.43, 51.54, 35.69, 33.58, 32.03, 26.20.

### Methyl 4-(2-(2-oxoethyl)phenylbutanoate (Compound 2.7.9)

To a solution of compound 2.7.8 (1 equiv.) in anhydrous CH₂Cl₂ under an argon atmosphere at 0° C. was added Dess-Martin periodinane (1.8 equiv.). The reaction was brought to room temperature over a period of 1 hour. The reaction mixture was quenched by adding a mixture of Na₂S₂O₃ (10% in H₂O) and saturated Sodium bicarbonate (1:1) and extracted with Et₂O, washed with saturated Sodium bicarbonate and brine, and dried over MgSO₄. The mixture was concentrated under reduced pressure. The resulting crude residue was chromatographed on silica gel (10-40% EtOAc/hexane) to afford pure title compounds in 88% yield as light yellow oil. ¹H NMR (400 MHz, CDCl₃) δ 9.72 (t, J=1.9 Hz, 1H), 7.28-7.11 (m, 4H), 3.74 (d, J=1.9 Hz, 2H), 3.67 (s, 3H), 2.61 (t, J=7.8 Hz, 2H), 2.37 (t, J=7.1 Hz, 2H), 1.87 (quintet, J=7.8 Hz, 2H). ¹³C NMR (100 MHz, CDCl₃) δ 199.40, 173.65, 140.45, 130.89, 130.13, 129.80, 127.84, 126.76, 51.54, 47.96, 33.39, 32.24, 25.87.

### Methyl 4-(2-((2Z,5Z,8Z)-tetradeca-2,5,8-trien-1-yl)phenyl)butanoate (Compound 2.7.10)

Compound 2.7.10 was synthesized in a similar manner to compound 2.2.2a. The resulting crude product was chromatographed on silica gel (0-40% Et₂O/hexane)) to afford pure title compound in 90% yield as light yellow oil. ¹H NMR (400 MHz, CDCl₃) δ 7.20-7.12 (m, 4H), 5.57-5.31 (m, 6H), 3.67 (s, 3H), 3.43 (d, J=5.6 Hz, 2H), 2.94 (t, J=5.2 Hz, 2H), 2.83 (t, J=5.6 Hz, 2H), 2.67 (m, 2H), 2.38 (t, J=7.3 Hz, 2H), 2.05 (q, J=7.3 Hz, 2H), 1.87 (quintet, J=7.8 Hz, 2H) 1.41-1.24 (m, 6H), 0.88 (t, J=6.7 Hz, 3H).

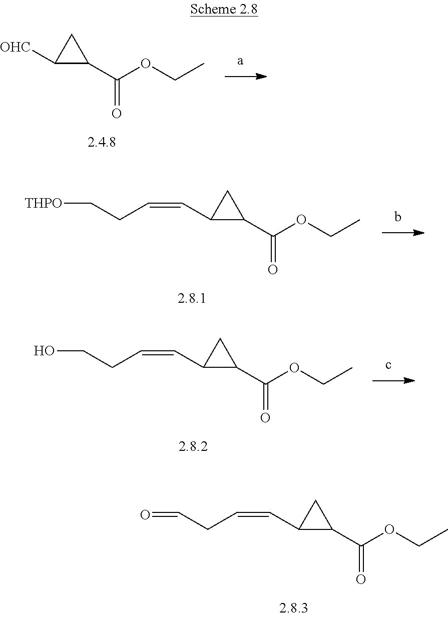

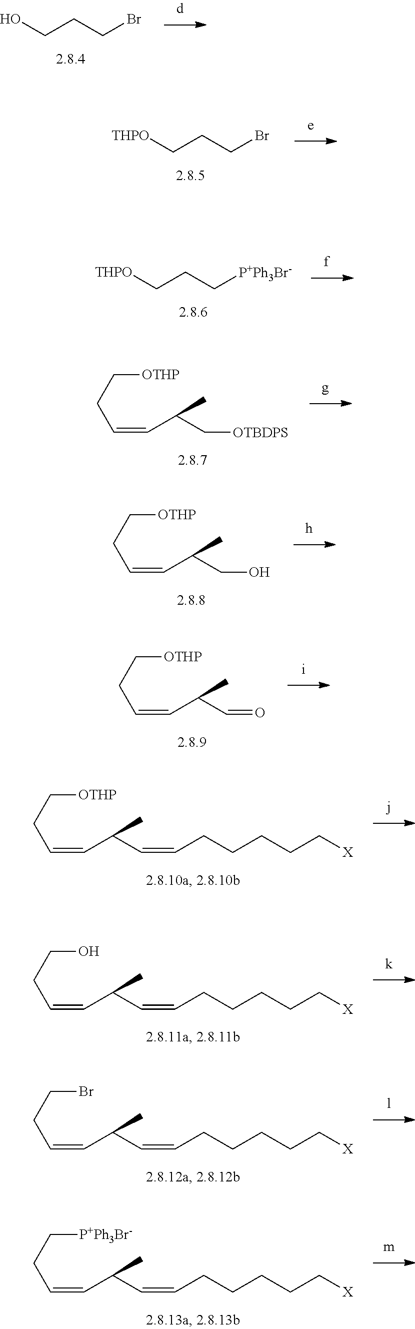

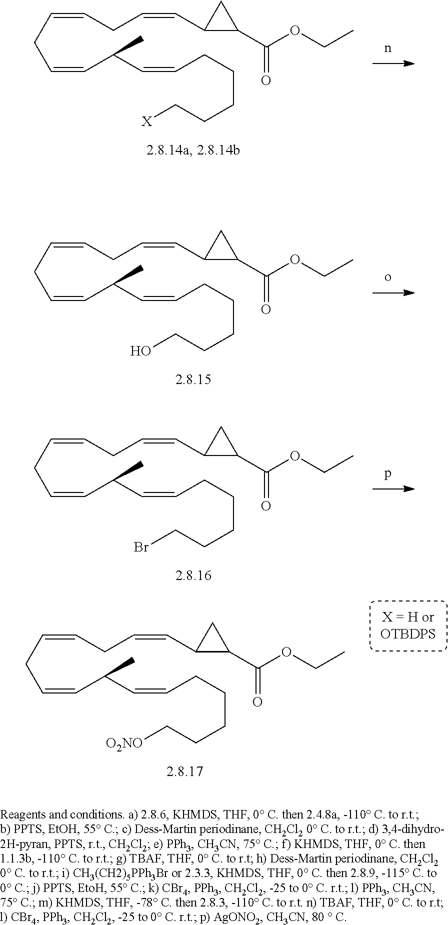

Experimental procedures for the syntheses of all compounds depicted in scheme 2.8 were similar to those described for related syntheses in scheme 2.3

### Ethyl 2-((S,1Z,4Z,7Z,10Z)-9-methylheptadeca-1,4,7,10-tetraen-1-yl)cyclopropane-1-carboxylate (Compound 2.8.14a)

This compound was synthesized in a manner similar to the one described for 2.3.4. The final compound was isolated as a colorless oil.

### Ethyl 2-((S,1Z,4Z,7Z,10Z)-9-methyl-16-(nitrooxy)hexadeca-1,4,7,10-tetraen-1-yl)cyclopropane-1-carboxylate (Compound 2.8.17)

This compound was synthesized in a manner similar to the one described for 2.3.7. The final compound was isolated as a colorless oil. ¹H NMR (500 MHz, CDCl₃) δ 5.44-5.34 (m, 3H), 5.31-5.20 (m, 4H), 4.82 (t, J=10.1 Hz, 1H), 4.44 (t, J=6.7 Hz, 2H), 4.14 (q, J=7.0 Hz, 2H), 3.45 (ddq, J=7.4, 7.3, 7.3 Hz, 1H), 2.98-2.90 (m, 2H), 2.90-2.78 (m, 2H), 2.21-2.14 (m, 1H), 2.12-2.05 (m, 2H), 1.72 (dt, J=7.7, 7.0 Hz, 2H), 1.61 (dt, J=8.7, 4.7 Hz, 1H), 1.41 (d, J=253.3 Hz, 5H), 1.27 (t, J=7.2 Hz, 3H), 1.02 (d, J=6.7 Hz, 3H), 0.91 (dddd, J=10.9, 8.6, 5.8 Hz, 1H).

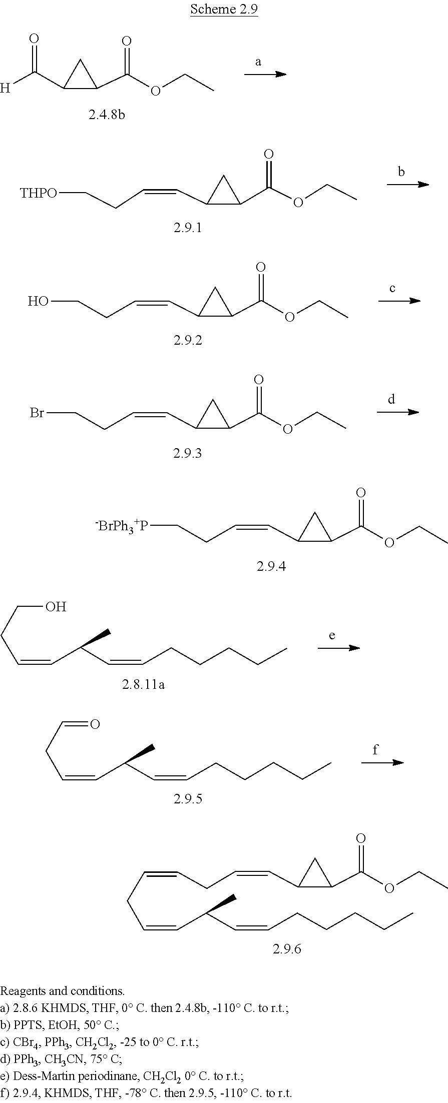

Experimental procedures for the syntheses of all compounds depicted in scheme 2.9 were similar to those described for related syntheses in scheme 2.3.

### Ethyl 2-((S,1Z,4Z,7Z,10Z)-9-methylhexadeca-1,4,7,10-tetraen-1-yl)cyclopropane-1-carboxylate (Compound 2.9.6)

This compound was synthesized in a manner similar to the one described for 2.3.4. The final compound was isolated as a colorless oil. ¹H NMR (500 MHz, CDCl₃) δ 5.49 (dt, J=10.8 Hz, J=7.3 Hz, 1H), 5.45-5.36 (m, 3H), 5.33-5.22 (m, 4H), 4.15 (qd, J=7.1 Hz, J=0.4 Hz, 2H), 3.48 (sextet, J=6.6 Hz, 1H), 3.01-2.81 (m, 4H), 2.15-2.04 (m, 3H) [overlapping patterns i.e., 2.13 (quintet, J=8.5 Hz, 1H), 2.11-2.04 (m, 2H)], 1.95 (q, J=6.3 Hz, 1H), 1.41-1.21 (m, 11H) [overlapping patterns i.e., 1.37 (quintet, J=7.1 Hz, 2H), 1.35-1.21 (m, 9H)], 1.03 (d, J=6.8 Hz, 3H), 0.91 (t, J=7.1 Hz, 3H).

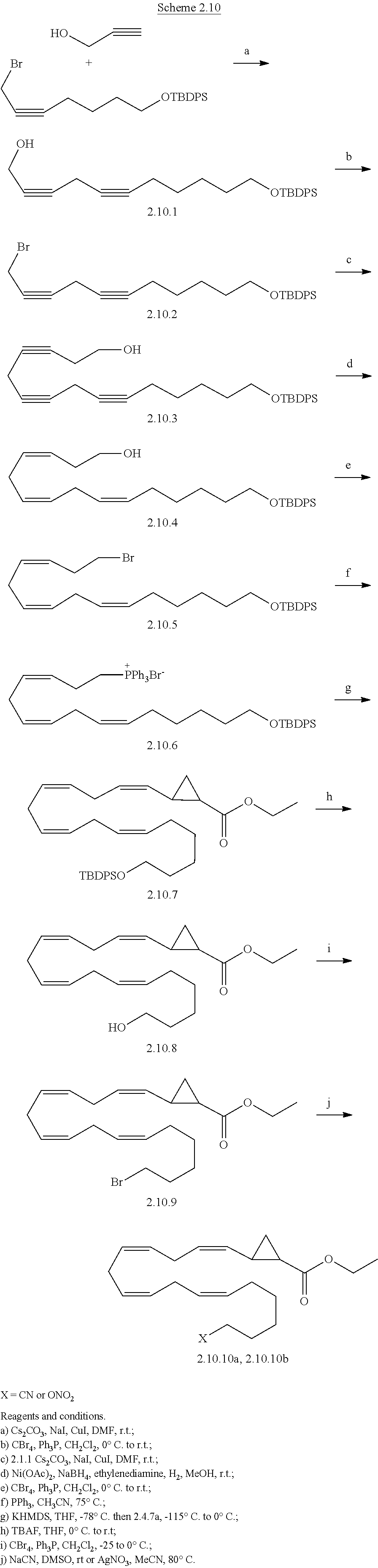

Experimental procedures for the syntheses of all compounds depicted in scheme 2.10 were similar to those described for related syntheses in scheme 2.4.

### Ethyl 2-((1Z,4Z,7Z,10Z)-16-cyanohexadeca-1,4,7,10-tetraen-1-yl)cyclopropane-1-carboxylate (Compound 2.10.10a)

This compound was synthesized in a manner similar to the one described for 2.3.7a. The final compound was isolated as a colorless oil. ¹H NMR (500 MHz, CDCl₃) δ 5.47-5.30 (m, 7H), 4.84 (tt, J=10.6 Hz, J=1.1 Hz, 1H), 4.15 (qd, J=7.1 Hz, J=0.6 Hz, 2H), 3.02-2.90 (m, 2H), 2.86 (t, J=4.9 Hz, 2H), 2.82 (t, J=4.8 Hz, 2H), 2.35 (t, J=7.1 Hz, 2H), 2.22-2.14 (m, 1H), 2.10 (q, J=5.8 Hz, 2H), 1.68 (quintet, J=7.5 Hz, 2H), 1.62 (quintet of d, J=4.0 Hz, J=1.1 Hz, 1H)], 1.52-1.35 (m, 5H) [overlapping patterns i.e., 1.48 (quintet of d, J=4.4 Hz, J=0.5 Hz, 1H), 1.45-1.35 (m, 4H)], 1.28 (t, J=7.5 Hz, 3H), 0.92 (ddd, J=8.2 Hz, J=6.3 Hz, J=4.3 Hz, 1H).

### Ethyl 2-((1Z,4Z,7Z,10Z)-16-(nitrooxy)hexadeca-1,4,7,10-tetraen-1-yl)cyclopropane-1-carboxylate (Compound 2.10.10b)

This compound was synthesized in a manner similar to the one described for 2.3.7b. The final compound was isolated as a colorless oil. ¹H NMR (500 MHz, CDCl₃) δ 5.47-5.35 (m, 7H), 4.84 (tt, J=10.4 Hz, J=0.6 Hz, 1H), 4.46 (t, J=6.7 Hz, 2H), 4.15 (qd, J=7.1 Hz, J=0.4 Hz, 2H), 3.00-2.93 (m, 2H), 2.87 (t, J=4.9 Hz, 2H), 2.83 (t, J=4.8 Hz, 2H), 2.23-2.16 (m, 1H), 2.14-2.02 (m, 2H), 1.75 (quintet, J=7.5 Hz, 2H), 1.63 (quintet of d, J=4.0 Hz, J=1.1 Hz, 1H), 1.49-1.37 (m, 5H), 1.28 (t, J=7.1 Hz, 3H), 0.93 (ddd, J=8.2 Hz, J=6.3 Hz, J=4.3 Hz, 1H).

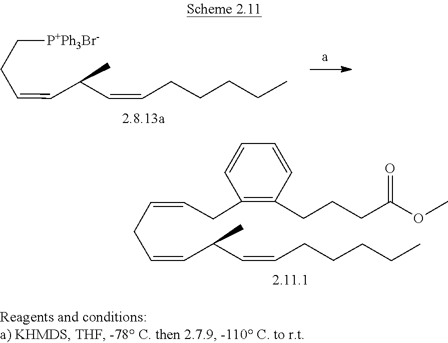

### Methyl 4-(2-((S,2Z,5Z,8Z)-7-methyltetradeca-2,5,8-trien-1-yl)phenyl)butanoate

This compound was synthesized in a manner similar to the one described for 2.3.4 The final compound was isolated as a colorless oil ¹H NMR (500 MHz, CDCl₃) δ 7.15 (s, 4H), 5.56-5.44 (m, 2H), 5.35-5.20 (m, 4H), 3.67 (s, 3H), 3.49 (ddq, J=8.1, 7.5, 6.5 Hz, 1H), 3.42 (d, J=5.4 Hz, 2H), 3.02-2.88 (m, 2H), 2.66 (t, J=8.1 Hz, 2H), 2.38 (t, J=7.4 Hz, 2H), 2.10-2.01 (m, 2H), 1.99-1.87 (m, 2H), 1.34 (d, J=6.8 Hz, 2H), 1.28 (m, 4H), 1.02 (d, J=6.7 Hz, 3H), 0.87 (t, J=6.8 Hz, 3H).

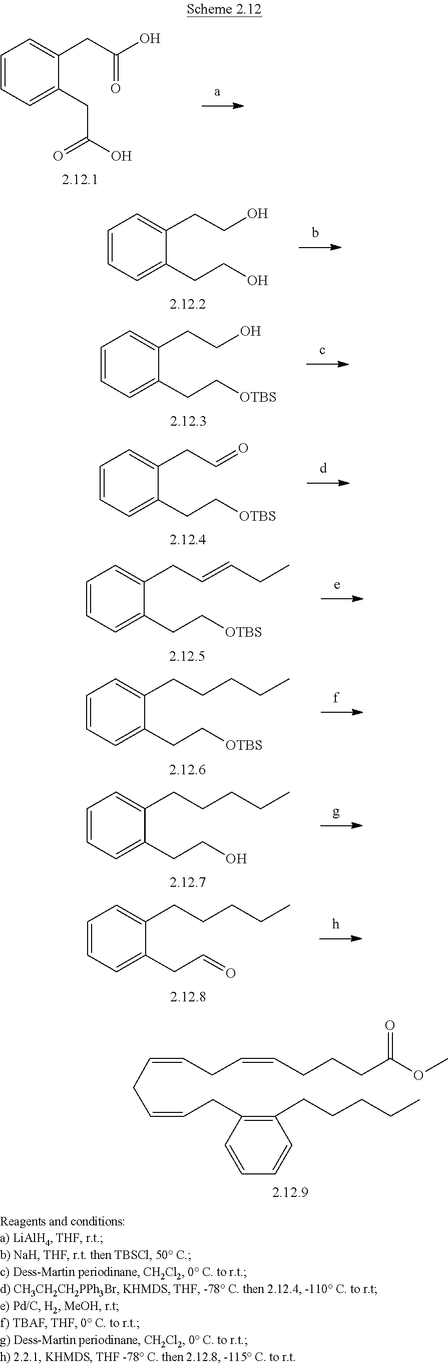

Experimental procedures for the syntheses of all compounds depicted in scheme 2.12 were similar to those described for related syntheses in schemes 2.2 and 2.7.

### Methyl (5Z,8Z,11Z)-13-(2-pentylphenyl)trideca-5,8,11-trienoate (Compound 2.12.9)

This compound was synthesized in a manner similar to the one described for 2.3.7a. The final compound was isolated as a colorless oil. ¹H NMR (500 MHz, CDCl₃) δ 7.19-7.13 (m, 4H), 5.58-5.35 (m, 6H), 3.68 (s, 3H), 3.44 (d, J=6.5 Hz, 2H), 2.96 (t, J=6.5 Hz, 2H), 2.84 (t, J=6.5 Hz, 2H), 2.63 (t, J=8.0 Hz, 2H), 2.33 (t, J=7.5 Hz, 2H), 2.13 (q, J=6.9 Hz, 2H), 1.73 (quintet, J=7.4 Hz, 2H), 1.60 (quintet, J=7.5 Hz, 2H), 1.42-1.34 (m, 4H), 0.92 (t, J=7.2 Hz, 3H).

### 3. Synthesis of Endocannabinoid-Like Amine/Alcohol Precursors

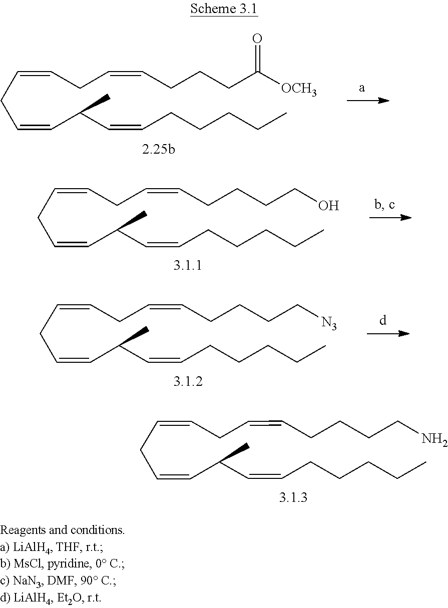 [SMILES: CCCCC/C=C\[C@H](C)/C=C\C/C=C\C/C=C\CCCC(=O)OC ; CCCCC/C=C\[C@H](C)/C=C\C/C=C\C/C=C\CCCCO ; CCCCC/C=C\[C@H](C)/C=C\C/C=C\C/C=C\CCCCN=[N+]=[N-] ; CCCCC/C=C\[C@H](C)/C=C\C/C=C\C/C=C\CCCCN]

### (S,5Z,8Z,11Z,14Z)-13-Methylicosa-5,8,11,14-tetraen-1-ol (Compound 3.1.1)

To a solution of compound 2.2.5b (1 equiv.) in anhydrous Et₂O [0.1 M] under an argon atmosphere at room temperature was added LiAlH₄ (2 equiv.). The reaction was left for 1 hour. The reaction mixture was quenched by Sat. NH₄Cl, extracted with Et₂O, and dried over MgSO₄. The mixture was concentrated under reduced pressure. The resulting crude residue was chromatographed on silica gel (10-40% EtOAc/hexane) to afford pure title compound in 85% yield as light yellow oil. ¹H NMR (500 MHz, CDCl₃) δ 5.44-5.31 (m, 4H), 5.31-5.19 (m, 4H), 3.65 (t, J=6.5 Hz, 2H), 3.46 (sextet, J=6.9 Hz, 1H), 2.92-2.74 (m, 4H), 2.15-2.00 (m, 5H), 1.59 (quintet, J=6.7 Hz, 2H), 1.44 (quintet, J=7.7 Hz, 2H), 1.39-1.21 (m, 6H), 1.01 (d, J=6.7 Hz, 3H), 0.88 (t, J=6.7 Hz, 3H).

### (S,5Z,8Z,11Z,14Z)-1-Azido-13-methylicosa-5,8,11,14-tetraene (Compound 3.1.2)

To a stirred solution of compound 3.1.1 (1 equiv.) in anhydrous pyridine [0.3 M] under an argon atmosphere at 0° C. was added mesyl chloride (1.5 equiv.). After stirring for 2 h, the reaction mixture was poured into cold water and extracted with Et₂O. The combined ether extracts were washed with 1 N sulfuric acid and sat. NaHCO₃ solution and concentrated under reduced pressure. The crude mesylate was dissolved in anhydrous DMF [0.1 M], and then a solution of sodium azide (5 equiv.) in anhydrous DMF [0.1 M] was added at room temperature. The reaction mixture was heated at 90° C. overnight. The mixture was cooled to room temperature and filtered through a pad of Celite. The filtrate was poured into cold water. The mixture was extracted with Et₂O, and dried over MgSO₄. The organic phase was concentrated under reduced pressure. The resulting crude product was chromatographed on silica gel (0-20% EtOAc/hexane) to afford pure title compound (50% yield) as a colorless oil. ¹H NMR (500 MHz, CDCl₃) δ 5.44-5.31 (m, 4H), 5.31-5.19 (m, 4H), 3.46 (sextet, J=7.0 Hz, 1H), 3.27 (t, J=6.9 Hz, 2H), 2.92-2.74 (m, 4H), 2.15-2.00 (m, 4H), 1.59 (quintet, J=6.7 Hz, 2H), 1.44 (quintet, J=7.7 Hz, 2H), 1.39-1.21 (m, 6H), 1.01 (d, J=6.7 Hz, 3H), 0.88 (t, J=6.7 Hz, 3H).

### (S,5Z,8Z,11Z,14Z)-13-Methylicosa-5,8,11,14-tetraen-1-amine (Compound 3.1.3)

Compound 3.1.3 was synthesized in a similar manner to compound 3.1.1. The resulting crude product was chromatographed on silica gel (0-10% MeOH/CH₂Cl₂) to afford pure title compound in 71% yield as light yellow oil. ¹H NMR (500 MHz, CDCl₃) δ 5.44-5.31 (m, 4H), 5.31-5.19 (m, 4H), 3.46 (sextet, J=6.9 Hz, 1H), 2.91-2.77 (m, 4H), 2.72 (t, J=6.7 Hz, 2H), 2.15-2.00 (m, 4H), 1.49 (quintet, J=6.7 Hz, 2H), 1.44-1.23 (m, 8H) [overlapping patterns i.e. 1.40 (quintet, J=7.7 Hz, 2H), 1.39-1.23 (m, 6H)], 1.01 (d, J=6.7 Hz, 3H), 0.88 (t, J=6.7 Hz, 3H).

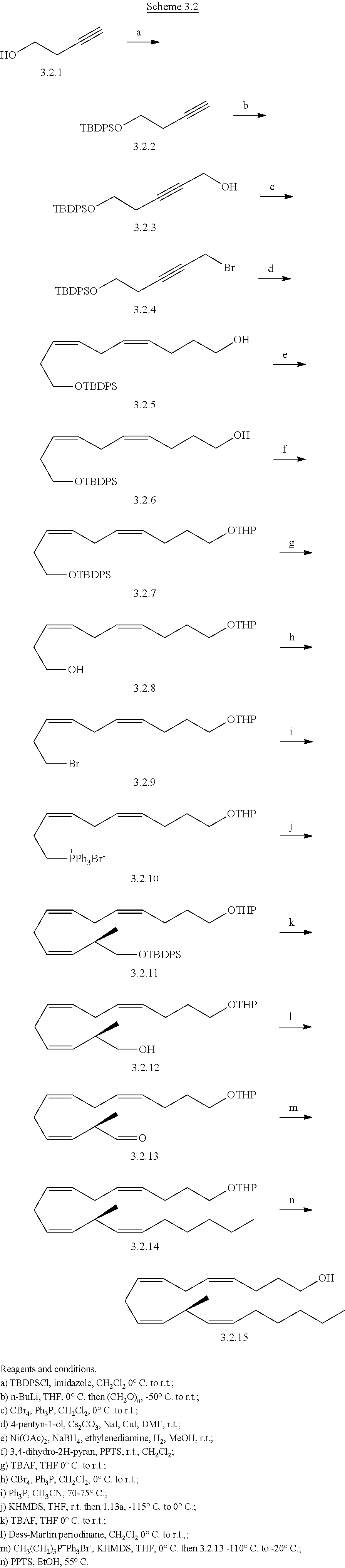

The first 3 steps of the synthesis leading to intermediate bromide 3.2.4 along with the spectroscopic data for the compounds are identical to those reported in Papahatjis et al. (2010), Chem. Eur. J, 16, 4091-4099 the content of which is hereby incorporated by reference.

### 10-((tert-Butyldiphenylsilyl)oxy)deca-4,7-diyn-1-ol (Compound 3.2.5)

To a stirred mixture of Cs₂CO₃ (1 equiv.), NaI (1 equiv.) and CuI (1 equiv.) in anhydrous DMF [0.5 M] under an argon atmosphere at room temperature was added a solution of 4-pentyn-1-ol (1.2 equiv.) in anhydrous DMF. The suspension was stirred for 30 min. A solution of ((5-bromopent-3-yn-1-yl)oxy)(tert-butyl)diphenylsilane (1 equiv.) in anhydrous DMF was added and the reaction was kept at room temperature for 2 h. The reaction mixture was then cooled to 0° C., diluted with Et₂O, quenched by Sat. NH₄Cl and dissolved in H₂O. The mixture was brought to room temperature and extracted with Et₂O. The combined organic extracts were washed with brine, dried over MgSO₄ and concentrated under reduced pressure. The resulting crude residue was chromatographed on silica gel (10-35% Et₂O/hexane) to afford pure title compound in 95% yield as light yellow oil. ¹H NMR (500 MHz, CDCl₃) δ 7.68 (dd, t, J=7.8 Hz, J=1.3 Hz, 4H), 7.45-7.36 (m, 6H), 3.77-3.70 (m, 4H) [overlapping patterns i.e. 3.75 (t, J=7.1 Hz, 2H), 3.72 (t, J=6.3 Hz, 2H)], 3.08 (t, J=2.3 Hz, 2H), 2.43 (tt, J=7.1 Hz, J=2.3 Hz, 2H), 2.27 (tt, J=7.1 Hz, J=2.3 Hz, 2H), 2.04 (s, 1H), 1.72 (quintet, J=6.3 Hz, 2H), 1.05 (s, 9H).

### (4Z,7Z)-10-((Tert-butyldiphenylsilyl)oxy)deca-4,7-dien-1-ol (Compound 3.2.6)

To a stirred mixture of Ni(OAc)₂ (1.7 equiv.) in anhydrous MeOH [0.06 M] under an argon atmosphere at 0° C. was added NaBH₄ (2 equiv.). The suspension was stirred for 15 min. Ethylenediamine (10 equiv.) was added and then the argon atmosphere was changed to H2 after 15 min. A solution of 10-((tert-butyldiphenylsilyl)oxy)deca-4,7-diyn-1-ol (1 equiv.) in anhydrous MeOH was added and the reactions was kept at room temperature for 1.5 h. The reaction suspension was filtrated through a pad of Celite and washed with Et₂O. The filtrate was concentrated under reduced pressure and extracted with Et₂O. The combined organic extracts were washed with brine, dried over MgSO₄ and concentrated under reduced pressure. The resulting crude residue was chromatographed on silica gel (5-20% Et₂O/hexane) to afford pure title compound in 85% yield as light yellow oil. ¹H NMR (500 MHz, CDCl₃) δ 7.68 (dd, t, J=7.8 Hz, J=1.3 Hz, 4H), 7.45-7.36 (m, 6H), 5.45-5.31 (m, 4H), 3.70-3.60 (m, 4H) [overlapping patterns i.e. 3.66 (t, J=6.9 Hz, 2H), 3.63 (t, J=6.2 Hz, 2H)], 2.76 (t, J=5.5 Hz, 2H), 2.34 (q, J=6.7 Hz, 2H), 2.13 (q, J=6.8 Hz, 2H), 1.63 (quintet, J=6.6 Hz, 2H), 1.34 (br s, 1H), 1.05 (s, 9H).

### tert-Butyldiphenyl(((3Z,6Z)-10-((tetrahydro-2H-pyran-2-yl)oxy)deca-3,6-dien-1-yl)oxy)silane (Compound 3.2.7)

Compound 3.2.7 was synthesized in a similar manner to compound 2.3.2. The resulting crude product was chromatographed on silica gel (5-20% Et₂O/hexane) to afford pure title compound in 91% yield as colorless oil. ¹H NMR (500 MHz, CDCl₃) δ 7.67 (dd, t, J=7.9 Hz, J=1.4 Hz, 4H), 7.45-7.35 (m, 6H), 5.45-5.28 (m, 4H), 4.57 (t, J=4.1 Hz, 1H), 3.90-3.83 (m, 1H), 3.74 (dt, J=9.6 Hz, J=6.7 Hz, 1H), 3.66 (t, J=6.8 Hz, 2H), 3.53-3.46 (m, 1H), 3.38 (dt, J=9.6 Hz, J=6.7 Hz, 1H), 2.74 (t, J=5.5 Hz, 2H), 2.33 (q, J=6.7 Hz, 2H), 2.13 (nonet, J=7.7 Hz, 2H), 1.87-1.77 (m, 1H), 1.75-1.62 (m, 3H) [overlapping patterns i.e. 1.75-1.62 (m, 1H), 1.66 (quintet, J=6.6 Hz, 2H), 1.61-1.46 (m, 4H), 1.04 (s, 9H).

### (3Z,6Z)-10-((Tetrahydro-2H-pyran-2-yl)oxy)deca-3,6-dien-1-ol (Compound 3.2.8)

To a stirred solution of tert-butyldiphenyl(((3Z,6Z)-10-((tetrahydro-2H-pyran-2-yl)oxy)deca-3,6-dien-1-yl)oxy)silane (1 equiv.) in anhydrous THF [0.05 M] under an argon atmosphere at 0° C. was added tetra-n-butylammonium fluoride (1.3 equiv. 1 M solution in THF). The reaction was kept at 0° C. for 10 minutes and then at room temperature for 1.5 hours. The reaction mixture was diluted by Et₂O, quenched by Sat. NH₄Cl, extracted with Et₂O. The combined organic extracts were washed with brine, dried over MgSO₄ and concentrated under reduced pressure. The resulting crude residue was chromatographed on silica gel (20-70% EtOAc/hexane) to afford pure title compound in 93% yield as light yellow oil. ¹H NMR (500 MHz, CDCl₃) δ 5.58-5.51 (m, 1H), 5.45-5.32 (m, 3H), 4.57 (t, J=4.0 Hz, 1H), 3.90-3.83 (m, 1H), 3.74 (dt, J=9.6 Hz, J=6.7 Hz, 1H), 3.65 (t, J=6.2 Hz, 2H), 3.53-3.46 (m, 1H), 3.40 (dt, J=9.6 Hz, J=6.7 Hz, 1H), 2.83 (t, J=6.8 Hz, 2H), 2.36 (q, J=6.8 Hz, 2H), 2.16 (nonet, J=7.2 Hz, 2H), 1.87-1.77 (m, 1H), 1.75-1.62 (m, 3H) [overlapping patterns i.e. 1.75-1.62 (m, 1H), 1.66 (quintet, J=6.6 Hz, 2H), 1.61-1.46 (m, 6H).

### 2-(((4Z,7Z)-10-Bromodeca-4,7-dien-1-yl)oxy)tetrahydro-2H-pyran (Compound 3.2.9)

Compound 3.2.9 was synthesized in a similar manner to compound 2.3.6. The volatile materials were removed under reduced pressure and the resulting residue was chromatographed on silica gel (0-20% diethyl Et₂O-hexane) to afford title bromide in 96% yield as light yellow oil. ¹H NMR (500 MHz, CDCl₃) δ 5.56-5.48 (m, 1H), 5.45-5.32 (m, 3H), 4.57 (t, J=4.0 Hz, 1H), 3.90-3.83 (m, 1H), 3.75 (dt, J=9.6 Hz, J=6.7 Hz, 1H), 3.53-3.46 (m, 1H), 3.43-3.34 (m, 3H) [overlapping patterns i.e. 3.40 (dt, J=9.6 Hz, J=6.7 Hz, 1H), 3.37 (t, J=7.1 Hz, 2H),)], 2.80 (t, J=7.1 Hz, 2H), 2.65 (q, J=7.2 Hz, 2H), 2.16 (nonet, J=7.2 Hz, 2H), 1.87-1.77 (m, 1H), 1.75-1.62 (m, 3H) [overlapping patterns i.e. 1.75-1.62 (m, 1H), 1.66 (quintet, J=6.6 Hz, 2H)], 1.61-1.46 (m, 4H).

### Triphenyl((3Z,6Z)-10-((tetrahydro-2H-pyran-2-yl)oxy)deca-3,6-dien-1-yl)phosphonium Bromide (Compound 3.2.10)

Compound 3.2.10 was synthesized in a similar manner to compound 2.3.3. The resulting residue was chromatographed on silica gel (2-8% MeOH/CH₂Cl₂) to afford title phosphonium salt in 95% yield as light brown gum. ¹H NMR (500 MHz, CDCl₃) δ 7.89 (dd, J=12.6 Hz, J=1.1 Hz, 6H), 7.79 (t, J=6.5 Hz, 3H), 7.70 (td, J=7.8 Hz, J=3.2 Hz, 6H), 5.65-5.58 (m, 1H), 5.40-5.30 (m, 2H), 5.21-5.13 (m, 1H), 4.52 (t, J=4.0 Hz, 1H), 4.00 (dt, J=12.3 Hz, J=7.8 Hz, 2H), 3.88-3.81 (m, 1H), 3.68 (dt, J=9.6 Hz, J=6.7 Hz, 2H), 3.52-3.44 (m, 1H), 3.33 (dt, J=9.6 Hz, J=6.7 Hz, 2H), 2.53 (t, J=7.3 Hz, 2H), 2.51-2.43 (m, 2H), 1.97 (nonet, J=7.4 Hz, 2H), 1.87-1.77 (m, 1H), 1.75-1.62 (m, 1H), 1.63-1.46 (m, 4H) [overlapping patterns i.e. 1.66 (quintet, J=7.0 Hz, 2H)], 1.61-1.46 (m, 2H).

### tert-Butyl(((2R,3Z,6Z,9Z)-2-methyl-13-((tetrahydro-2H-pyran-2-yl)oxy)trideca-3,6,9-trien-1-yl)oxy)diphenylsilane (Compound 3.2.11)

To a stirred solution of Triphenyl((3Z,6Z)-10-((tetrahydro-2H-pyran-2-yl)oxy)deca-3,6-dien-1-yl)phosphonium bromide (3 equiv.) in anhydrous THF [0.17 M] at −78° C. under an argon atmosphere was added potassium bis(trimethylsilyl)amide (2 equiv.) The mixture was stirred for 30 min to ensure complete formation of the orange ylide, and then it was cooled to −115° C. Subsequently, a solution of (S)-3-((tert-butyldiphenylsilyl)oxy)-2-methylpropanal (1 equiv.) in anhydrous THF was added dropwise. The reaction mixture was stirred for 25 minutes at −115° C., and then warmed to 0° C. for 2 h. The reaction mixture was then cooled to −78° C., diluted with Et₂O and quenched with Sat. NaHCO₃. The mixture was brought to room temperature and extracted with Et₂O. The combined organic extracts were washed with brine, dried over MgSO₄ and concentrated under reduced pressure. The residue was chromatographed on silica gel (0-10% Et₂O/hexane) to afford title compound in 70% yield as colorless oil. ¹H NMR (500 MHz, CDCl₃) δ 7.67 (dd, t, J=7.8 Hz, J=1.2 Hz, 4H), 7.45-7.35 (m, 6H), 5.45-5.28 (m, 5H), 5.20 (m as t, J=9.5 Hz, 1H), 4.57 (t, J=4.0 Hz, 1H), 3.86 (td, J=7.7 Hz, J=2.2 Hz, 1H), 3.75 (dt, J=9.6 Hz, J=6.7 Hz, 1H), 3.54-3.43 (m, 3H), 3.39 (dt, J=9.6 Hz, J=6.7 Hz, 1H), 2.82-2.77 (m, 2H), 2.77-2.68 (m, 2H), 2.15 (nonet, J=8.0 Hz, 2H), 1.87-1.77 (m, 1H), 1.75-1.62 (m, 3H), [overlapping patterns i.e. 1.75-1.63 (m, 1H), 1.67 (quintet, J=7.1 Hz, 2H)], 1.61-1.46 (m, 5H), 1.05 (s, 9H), 1.01 (d, J=6.7 Hz, 3H).

### (2R,3Z,6Z,9Z)-2-Methyl-13-((tetrahydro-2H-pyran-2-yl)oxy)trideca-3,6,9-trien-1-ol (Compound 3.2.12)

To a stirred solution of tert-butyl(((2R,3Z,6Z,9Z)-2-methyl-13-((tetrahydro-2H-pyran-2-yl)oxy)trideca-3,6,9-trien-1-yl)oxy)diphenylsilane (1 equiv.) in anhydrous THF [0.05 M] under an argon atmosphere at 0° C. was added tetra-n-butylammonium fluoride (1.3 equiv. 1 M solution in THF). The reaction was kept at 0° C. for 10 minutes and then at room temperature for 1.5 hours. The reaction mixture was diluted by Et₂O, quenched by Sat. NH₄Cl, extracted with Et₂O and the combined organic extracts were washed with brine, dried over MgSO₄ and concentrated under reduced pressure. The resulting crude residue was chromatographed on silica gel (10-50% EtOAc/hexane) to afford pure title compound in 90% yield as colorless oil. ¹³C NMR (125 MHz, CDCl₃) δ 132.51, 130.02, 129.61, 128.53, 128.15, 127.94, 98.88, 67.61, 66.97, 62.36, 34.91, 30.75, 29.63, 26.03, 25.62, 25.47, 23.93, 19.66, 16.97.

### (2R,3Z,6Z,9Z)-2-Methyl-13-((tetrahydro-2H-pyran-2-yl)oxy)trideca-3,6,9-trienal (Compound 3.2.13)

Compound 3.2.13 was synthesized in a similar manner to compound 2.2.4b. The sensitive crude aldehyde was used for the next step immediately without further purification.

### 2-(((S,4Z,7Z,10Z,13Z)-12-Methylnonadeca-4,7,10,13-tetraen-1-yl)oxy)tetrahydro-2H-pyran (Compound 3.2.14)

To a stirred solution of hexyltriphenylphosphonium bromide (3 equiv.) in anhydrous THF [0.2 M] at −78° C. under an argon atmosphere was added potassium bis(trimethylsilyl)amide (2 equiv.) The mixture was stirred for 30 min to ensure complete formation of the orange ylide, and then it was cooled to −115° C. Subsequently, a solution of the above crude aldehyde compound 3.2.13 (1 equiv.) in anhydrous THF was added dropwise. The reaction mixture was stirred for 25 minutes at −115° C., and then warmed to 0° C. for 2 h. The reaction mixture was then cooled to −115° C. and quenched with a saturated aqueous sodium bicarbonate solution. The mixture was warmed to room temperature, extracted with Et₂O and the combined organic extracts were washed with brine, dried over MgSO₄ and concentrated under reduced pressure. The residue was chromatographed on silica gel (0-10% diethyl ether-hexanes) to afford title compound in 85% yield as light yellow oil. ¹H NMR (500 MHz, CDCl₃) δ 5.54-5.30 (m, 4H), 5.30-5.19 (m, 4H), 4.57 (t, J=3.6 Hz, 1H), 3.86 (td, J=7.7 Hz, J=2.2 Hz, 1H), 3.75 (dt, J=9.6 Hz, J=6.7 Hz, 1H), 3.54-3.42 (m, 2H), 3.43-3.34 (m, 1H), 2.91-2.77 (m, 4H), 2.22-2.00 (m, 4H) [overlapping patterns i.e. 2.16 (nonet, J=7.9 Hz, 2H), 2.14-2.00 (m, 2H)], 1.88-1.76 (m, 1H), 1.75-1.63 (m, 3H), [overlapping patterns i.e. 1.75-1.63 (m, 1H), 1.67 (quintet, J=7.1 Hz, 2H)], 1.62-1.46 (m, 4H), 1.40-1.22 (m, 6H), 1.01 (d, J=6.7 Hz, 3H), 0.88 (t, J=7.0 Hz, 3H). ¹³C NMR (125 MHz, CDCl₃) δ 135.01, 134.15, 129.57, 128.29, 128.25, 128.24, 128.23 125.68, 98.85, 66.92, 62.30, 31.57, 30.76, 30.48, 29.67, 29.46, 27.50, 25.83, 25.58, 25.49, 23.89, 22.58, 22.03, 19.65, 14.07.

### (S,4Z,7Z,10Z,13Z)-12-Methylnonadeca-4,7,10,13-tetraen-1-ol (Compound 3.2.15)

Compound 3.2.15 was synthesized in a similar manner to compound 2.3.5. The solvent was evaporated under reduced pressure and the resulting residue was chromatographed on silica gel (20-40% Et₂O/hexane) to afford title alcohol in 70% yield as light yellow oil. ¹H NMR (500 MHz, CDCl₃) δ 5.45-5.31 (m, 4H), 5.30-5.19 (m, 4H), 3.86 (t, J=6.5 Hz, 2H), 3.46 (sextet, J=7.0 Hz, 1H), 3.26 (br s, 1H), 2.91-2.74 (m, 4H), 2.16 (dt, J=7.0 Hz, J=7.0 Hz, 2H), 2.10-2.00 (m, 2H)], 1.65 (quintet, J=6.7 Hz, 2H), 1.40-1.22 (m, 6H), 1.01 (d, J=6.7 Hz, 3H), 0.88 (t, J=6.6 Hz, 3H).

### 4. Synthesis of Amides and Ethanolamides

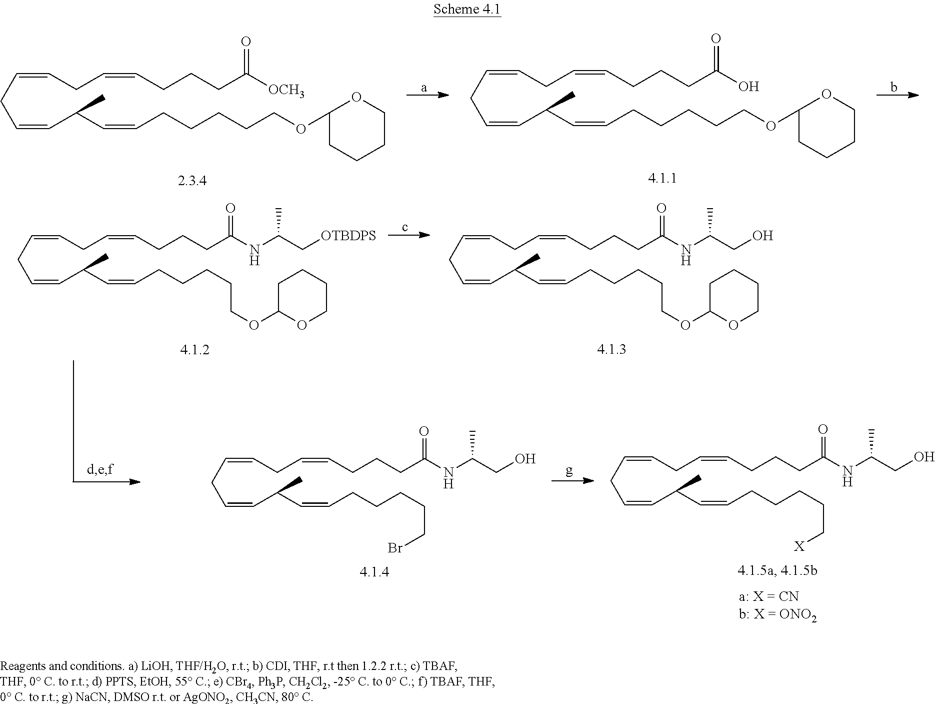

### (5Z,8Z,11Z,13S,14Z)-13-Methyl-20-((tetrahydro-2H-pyran-2-yl)oxy)icosa-5,8,11,14-tetraenoic Acid (Compound 4.1.1)

To a stirred solution of methyl (5Z,8Z,11Z,13S,14Z)-13-methyl-20-((tetrahydro-2H-pyran-2-yl)oxy)icosa-5,8,11,14-tetraenoate (1 equiv.) in THF (0.05 M) at room temperature under an argon atmosphere was added lithium hydroxide (5 equiv., 1 M in water). The reaction mixture was stirred at the same temperature overnight. the reaction mixture was acidified with 5% HCl to pH 3, and extracted with Et₂O. The combined organic extracts were washed with brine, dried over MgSO₄ and evaporated under reduced pressure. The resulting residue was chromatographed on silica gel (30-50% ethyl acetate-hexanes) to afford title carboxylic acid in 91% yield as a colorless oil.

### (5Z,8Z,11Z,13S,14Z)—N—((R)-1-((tert-Butyldiphenylsilyl)oxy)propan-2-yl)-13-methyl-20-((tetrahydro-2H-pyran-2-yl)oxy)icosa-5,8,11,14-tetraenamide (Compound 4.1.2)

A solution of methyl (5Z,8Z,11Z,13S,14Z)-13-methyl-20-((tetrahydro-2H-pyran-2-yl)oxy)icosa-5,8,11,14-tetraenoate (1 equiv.) and dried carbonyldiimidazole (4 equiv.) in anhydrous THF (0.06 M) at room temperature under an argon atmosphere, was stirred for 2 hours and then a solution of 2-((tert-butyldiphenylsilyl)oxy)ethan-1-amine (5 equiv.) in THF was added. The reaction mixture was stirred for 2 hour and then diluted with water and ethyl acetate. The organic phase was separated and the aqueous phase extracted with ethyl acetate. The combined organic layer was washed with brine, dried over MgSO₄ and concentrated under reduced pressure. The residue was chromatographed on silica gel (10-25% ethyl acetate-hexane) to afford title amide in 80% yield as light yellow oil.

### (S,5Z,8Z,11Z,14Z)-20-Bromo-N—((R)-1-hydroxypropan-2-yl)-13-methylicosa-5,8,11,14-tetraenamide (Compound 4.1.4)

To a stirred solution of (5Z,8Z,11Z,13S,14Z)—N—((R)-1-((tert-butyldiphenylsilyl)oxy)propan-2-yl)-13-methyl-20-((tetrahydro-2H-pyran-2-yl)oxy)icosa-5,8,11,14-tetraenamide (1 equiv.) in ethanol (0.12 M) at room temperature and under an argon atmosphere was added pyridinium p-toluenesulfonate (0.1 equiv.) and the mixture was heated to 55° C. and stirred at the same temperature for 4 hours. The solvent was evaporated under reduced pressure and the resulting residue was chromatographed on silica gel (10-30% ethyl acetate-hexanes) to afford pure (S,5Z,8Z,11Z,14Z)—N—((R)-1-((tert-butyldiphenylsilyl)oxy)propan-2-yl)-20-hydroxy-13-methylicosa-5,8,11,14-tetraenamide in 91% yield as light yellow oil.

A solution of the above alcohol (1 equiv.) in anhydrous dichloromethane (0.2 M) was cooled to −25° C. under an argon atmosphere and carbon tetrabromide (1.3 equiv.) and triphenylphosphine (1.3 equiv.) were added. The reaction mixture was stirred at −25° C. for 1 hour and then at 0° C. for 30 minutes. The volatile materials were removed under reduced pressure and the resulting residue was chromatographed on silica gel (0-20% ethyl acetate-hexanes) to afford pure (S,5Z,8Z,11Z,14Z)-20-bromo-N—((R)-1-((tert-butyldiphenylsilyl)oxy)propan-2-yl)-13-methylicosa-5,8,11,14-tetraenamide as light yellow oil.

The above protected bromide (1 equiv.) was dissolved in anhydrous tetrahydrofuran (0.05 M) and to the stirred mixture was added tetra-n-butylammonium fluoride (1.6 equiv. 1 M solution in tetrahydrofuran) at 0° C. under an argon atmosphere. Stirring was continued at 0° C. for 10 minutes and then for 1.5 hours at ambient temperature. The reaction was kept at 0° C. for 10 minutes and then at room temperature for 1.5 hours. The mixture was quenched by the addition of saturated aqueous NH₄Cl, extracted with diethyl ether and the combined organic extracts were washed with brine, dried over MgSO₄ and concentrated under reduced pressure. The residue was chromatographed on silica gel (30-60 ethyl acetate-hexanes) to afford pure title bromide a light yellow oil. ¹H NMR (500 MHz, CDCl₃) δ 5.57 (bs, 1H), 5.46-5.32 (m, 4H), 5.26 (m, 4H), 4.08 (ddq, J=7.3, 7.1, 6.3 Hz, 1H), 3.67 (dd, J=10.6, 2.7 Hz, 1H), 3.53 (dd, J=10.8, 6.2 Hz, 1H), 3.45 (dd, J=14.8, 7.8 Hz, 1H), 3.41 (t, J=6.8 Hz, 2H), 2.83 (dt, J=10.1, 5.7 Hz, 4H), 2.20 (t, J=8.1 Hz, 2H), 2.16-2.02 (m, 4H) [overlapping patterns i.e., 2.12 (t, J=7.5 Hz, 2H), 2.09-2.01 (m, 2H)], 1.86 (quintet, J=7.0 Hz, 2H), 1.73 (d, J=7.1 Hz, 2H), 1.58 (bs, 1H), 1.50-1.41 (m, 2H), 1.41-1.31 (m, 2H), 1.17 (d, J=6.9 Hz, 3H), 1.02 (d, J=6.7 Hz, 3H). Compound 4.1.3 was synthesized in a similar manner.

### (S,5Z,8Z,11Z,14Z)-20-Cyano-N—((R)-1-hydroxypropan-2-yl)-13-methylicosa-5,8,11,14-tetraenamide (Compound 4.1.5a)

Compound 4.1.5a was synthesized in a similar manner to compound 2.2.7a. Chromatography on silica gel (ethyl acetate) afforded the title nitrile in 66% yield as a colorless oil. ¹H NMR (500 MHz, CDCl₃) δ 5.65 (bs, 1H), 5.44-5.31 (m, 4H), 5.31-5.20 (m, 4H), 4.07 (ddq, J=7.3, 7.2, 6.4 Hz, 1H), 3.67 (dd, J=10.7, 4.3 Hz, 1H), 3.52 (dd, J=10.3, 5.4 Hz, 1H), 3.45 (dd, J=14.4, 7.2 Hz, 1H), 2.87-2.76 (m, 4H), 2.34 (t, J=7.1 Hz, 2H), 2.20 (t, J=7.6 Hz, 2H), 2.16-2.06 (m, 4H), 1.73 (t, J=7.8 Hz, 2H), 1.67 (t, J=7.5 Hz, 2H), 1.56 (bs, 1H), 1.51-1.43 (m, 2H), 1.40 (q, J=7.2 Hz, 2H), 1.17 (d, J=6.9 Hz, 3H), 1.02 (d, J=6.7 Hz, 3H).

### (S,6Z,9Z,12Z,15Z)-20-(((R)-1-Hydroxypropan-2-yl)amino)-8-methyl-20-oxoicosa-6,9,12,15-tetraen-1-yl Nitrate (Compound 4.1.5b)

Compound 4.1.5b was synthesized in a similar manner to compound 2.2.7b. Chromatography on silica gel (ethyl acetate) afforded title compound in 60% yield as a light yellow oil.

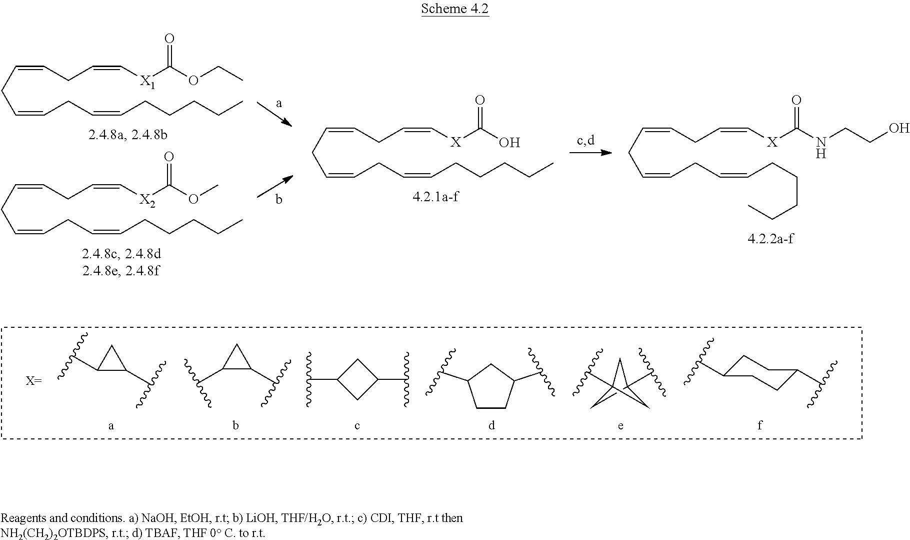

### 2-((1Z,4Z,7Z,10Z)-Hexadeca-1,4,7,10-tetraen-1-yl)cyclopropane-1-carboxylic Acid (Compound 4.2.1a)

To a stirred solution of Ethyl 2-((1Z,4Z,7Z,10Z)-hexadeca-1,4,7,10-tetraen-1-yl)cyclopropane-1-carboxylate (1 equiv.) in THF [0.15 M] under an argon atmosphere at room temperature was added a saturated solution of NaOH (5 equiv.) in EtOH. The suspension was stirred for 6 hours. The reaction mixture was quenched with 5% HCl and Sat. NH₄Cl, and extracted with EtOAc. The combined organic extracts were washed with brine, dried over MgSO₄ and evaporated under reduced pressure. The resulting crude was chromatographed on silica gel (5-30% EtOAc/hexane) to afford title carboxylic acid in 95% yield as colorless oil. ¹H NMR (500 MHz, CDCl₃) δ 11.36 (br s, 1H), 5.47-5.30 (m, 7H), 4.82 (tt, J=9.6 Hz, J=1.6 Hz, 1H), 3.51-3.39 (m, 2H), 3.02-2.90 (m, 2H), 2.84 (t, J=4.9 Hz, 2H), 2.81 (t, J=6.3 Hz, 2H), 2.30-2.22 (m, 1H), 2.05 (q, J=7.1 Hz, 2H), 1.61 (quintet of d, J=4.0 Hz, J=0.8 Hz, 1H), 1.47 (quintet, J=4.6 Hz, 1H), 1.40-1.24 (m, 6H), 0.99 (ddd, J=8.2 Hz, J=6.3 Hz, J=4.3 Hz, 1H), 0.88 (t, J=7.0 Hz, 3H). Compounds 4.2.1b was synthesized in a similar manner.

### 3-((1Z,4Z,7Z,10Z)-Hexadeca-1,4,7,10-tetraen-1-yl)bicyclo[1.1.1]pentane-1-carboxylic Acid (Compound 4.2.1d)

Compound 4.2.1d was synthesized in a similar manner as compound 4.1.1. The resulting crude was chromatographed on silica gel (5-30% EtOAc/hexane) to afford title carboxylic acid in 94% yield as colorless oil. ¹H NMR (500 MHz, CDCl₃) δ 10.07 (br s, 1H), 5.45-5.28 (m, 8H), 2.91 (t, J=6.7 Hz, 2H), 2.85-2.78 (m, 4H), 2.24 (s, 6H), 2.05 (q, J=7.0 Hz, 2H), 1.98 (quintet of d, J=4.0 Hz, J=0.8 Hz, 1H), 1.47 (quintet, J=4.6 Hz, 1H), 1.41-1.23 (m, 6H), 0.88 (t, J=7.0 Hz, 3H). Compounds 4.2.1c, 4.2.1e and 4.2.1f were synthesized in a similar manner.

### 2-((1Z,4Z,7Z,10Z)-Hexadeca-1,4,7,10-tetraen-1-yl)-N-(2-hydroxyethyl)cyclopropane-1-carboxamide (Compound 4.2.2a)

A mixture of 2-((1Z,4Z,7Z,10Z)-hexadeca-1,4,7,10-tetraen-1-yl)cyclopropane-1-carboxylic acid (1 equiv.) and dried carbonyldiimidazole (4 equiv.) in anhydrous THF [0.06 M] at room temperature under an argon atmosphere, was stirred for 2 hours and then a solution of 2-((tert-butyldiphenylsilyl)oxy)ethan-1-amine (5 equiv.) in THF was added. The reaction mixture was stirred for 1 hour and then diluted with EtOAc, quenched with H₂O and extracted with EtOAc. The combined organic layer was washed with brine, dried over MgSO₄ and concentrated under reduced pressure. The residue was chromatographed on silica gel (0-15% EtOAc/hexane) to afford protected amide in 91% yield as light yellow oil. To a stirred solution of above amide (1 equiv.) in anhydrous THF [0.05 M] under an argon atmosphere at 0° C. was added tetra-n-butylammonium fluoride (1.3 equiv. 1 M solution in THF). The reaction was kept at 0° C. for 10 minutes and then at room temperature for 1.5 hours. The reaction mixture was diluted by Et₂O, quenched by Sat. NH₄Cl, extracted with Et₂O and the combined organic extracts were washed with brine, dried over MgSO₄ and concentrated under reduced pressure. The resulting crude was chromatographed on silica gel (20-100% EtOAc/hexane) to afford pure title compound in 88% yield as light yellow oil. ¹H NMR (500 MHz, CDCl₃) δ 6.05 (br s, 1H), 5.47-5.30 (m, 7H), 4.82 (tt, J=10.5 Hz, J=1.5 Hz, 1H), 3.77-3.70 (m, 2H), 3.52-3.39 (m, 2H), 3.02-2.90 (m, 2H), 2.84 (t, J=5.6 Hz, 2H), 2.81 (t, J=6.1 Hz, 2H), 2.56 (br t, J=5.1 Hz, 1H), 2.22-2.15 (m, 1H), 2.05 (q, J=7.1 Hz, 2H), 1.44 (quintet of d, J=4.0 Hz, J=0.7 Hz, 1H), 1.40-1.23 (m, 6H), 0.89 (t, J=7.0 Hz, 3H), 0.84 (ddd, J=8.2 Hz, J=6.0 Hz, J=4.1 Hz, 1H). Compounds 4.2.2b-f were synthesized in a similar manner.

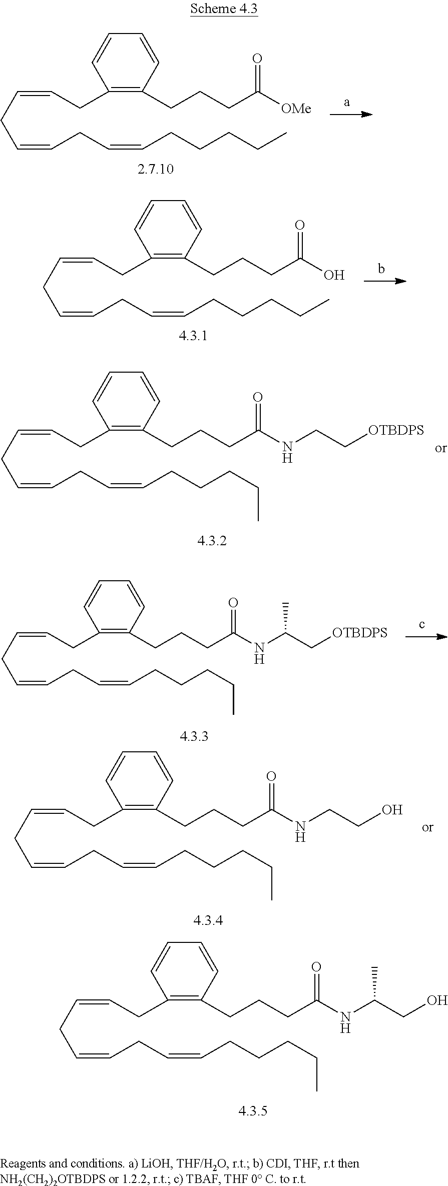

### 4-(2-((2Z,5Z,8Z)-Tetradeca-2,5,8-trien-1-yl)phenyl)butanoic Acid (Compound 4.3.1)

To a stirred solution of methyl 4-(2-((2Z,5Z,8Z)-tetradeca-2,5,8-trien-1-yl)phenyl)butanoate (1 equiv.) in THF (0.05 M) at room temperature under an argon atmosphere was added lithium hydroxide (5 equiv., 1 M in water). The reaction mixture was stirred at the same temperature overnight. The reaction mixture was quenched with 5% HCl and Sat. NH₄Cl, and extracted with EtOAc. The combined organic extracts were washed with brine, dried over MgSO₄ and evaporated under reduced pressure. The resulting crude was chromatographed on silica gel (5-30% EtOAc/hexane) to afford title carboxylic acid in 86% yield as colorless oil. ¹H NMR (400 MHz, CDCl₃) δ 10.50 (br s, 1H), 7.20-7.12 (m, 4H), 5.56-5.30 (m, 6H), 3.48-3.39 (m, 2H), 2.94 (t, J=5.0 Hz, 2H), 2.83 (t, J=5.8 Hz, 2H), 2.69 (dd, J=7.7 Hz, J=7.7 Hz, 2H), 2.42 (t, J=7.3 Hz, 2H), 2.05 (dt as q, J=6.8 Hz, 6.8 Hz, 2H), 1.94 (quintet, J=7.8 Hz, 2H), 1.44-1.24 (m, 6H), 0.89 (t, J=6.7 Hz, 3H). ¹³C NMR (100 MHz, CDCl₃) δ 179.1, 139.1, 138.6, 130.5, 129.2, 128.7, 128.7, 128.4, 127.6, 127.4, 126.4, 126.2, 65.8, 33.5, 31.9, 31.4, 30.5, 29.2, 27.2, 25.7, 25.6, 25.5, 22.5, 14.0.

### N-(2-((tert-Butyldiphenylsilyl)oxy)ethyl)-4-(2-((2Z,5Z,8Z)-tetradeca-2,5,8-trien-1-yl)phenyl)butanamide (Compound 4.3.2)

A mixture of 4-(2-((2Z,5Z,8Z)-tetradeca-2,5,8-trien-1-yl)phenyl)butanoic acid (1 equiv.) and dried carbonyldiimidazole (4 equiv.) in anhydrous THF [0.06 M] at room temperature under an argon atmosphere, was stirred for 2 hours and then a solution of 2-((tert-butyldiphenylsilyl)oxy)ethan-1-amine (5 equiv.) in THF was added. The reaction mixture was stirred for 1 hour and then diluted with EtOAc, quenched with H₂O and extracted with EtOAc. The combined organic layer was washed with brine, dried over MgSO₄ and concentrated under reduced pressure. The residue was chromatographed on silica gel (0-15% EtOAc/hexane) to afford title amide in 91% yield as light yellow oil. ¹H NMR (400 MHz, CDCl₃) δ 7.67-7.59 (m, 4H), 7.46-7.33 (m, 6H), 7.20-7.12 (m, 4H), 5.70 (t, J=4.8 Hz, 1H), 5.56-5.30 (m, 6H), 3.75 (t, J=5.0 Hz, 2H), 3.48-3.39 (m, 4H), 2.93 (t, J=5.0 Hz, 2H), 2.82 (t, J=5.8 Hz, 2H), 2.66 (dd, J=7.7 Hz, J=7.7 Hz, 2H), 2.17 (t, J=7.3 Hz, 2H), 2.05 (dt as q, J=6.8 Hz, 6.8 Hz, 2H), 1.91 (quintet, J=7.8 Hz, 2H), 1.40-1.20 (m, 6H), 1.06 (s, 9H), 0.89 (t, J=6.7 Hz, 3H).

### N—((R)-1-((tert-Butyldiphenylsilyl)oxy)propan-2-yl)-4-(2-((2Z,5Z,8Z)-tetradeca-2,5,8-trien-1-yl)phenyl)butanamide (Compound 4.3.3)

Compound 4.3.3 was synthesized in a similar manner as Compound 4.3.2. The residue was chromatographed on silica gel (0-15% EtOAc/hexane) to afford title amide in 90% yield as light yellow oil. ¹H NMR (400 MHz, CDCl₃) δ 7.67-7.59 (m, 4H), 7.46-7.31 (m, 6H), 7.20-7.12 (m, 4H), 5.55 (d, J=8.1 Hz, 1H), 5.54-5.30 (m, 6H), 4.17-4.06 (m, 1H), 3.67 (dd, J=10.1 Hz, J=4.0 Hz, 1H), 3.60 (dd, J=10.1 Hz, J=4.0 Hz, 1H), 3.41 (d, J=5.4 Hz, 2H), 2.93 (t, J=5.0 Hz, 2H), 2.82 (t, J=5.8 Hz, 2H), 2.65 (dd, J=7.7 Hz, J=7.7 Hz, 2H), 2.14 (t, J=7.3 Hz, 2H), 2.05 (dt as q, J=6.8 Hz, 6.8 Hz, 2H), 1.91 (quintet of d, J=7.3 Hz, J=2.2 Hz, 2H), 1.40-1.23 (m, 6H), 1.18 (d, J=6.6 Hz, 3H), 1.06 (s, 9H), 0.89 (t, J=6.7 Hz, 3H).

### N-(2-Hydroxyethyl)-4-(2-((2Z,5Z,8Z)-tetradeca-2,5,8-trien-1-yl)phenyl)butanamide (Compound 4.3.4)

To a stirred solution of N-(2-((tert-butyldiphenylsilyl)oxy)ethyl)-4-(2-((2Z,5Z,8Z)-tetradeca-2,5,8-trien-1-yl)phenyl)butanamide (1 equiv.) in anhydrous THF [0.05 M] under an argon atmosphere at 0° C. was added tetra-n-butylammonium fluoride (1.3 equiv. 1 M solution in THF). The reaction was kept at 0° C. for 10 minutes and then at room temperature for 1.5 hours. The reaction mixture was diluted by Et₂O, quenched by Sat. NH₄Cl, extracted with Et₂O and the combined organic extracts were washed with brine, dried over MgSO₄ and concentrated under reduced pressure. The resulting crude was chromatographed on silica gel (20-100% EtOAc/hexane) to afford pure title compound in 90% yield as light yellow oil. ¹H NMR (400 MHz, CDCl₃) δ 7.20-7.12 (m, 4H), 5.86 (br s, 1H), 5.56-5.30 (m, 6H), 3.71 (t, J=4.8 Hz, 2H), 3.45-3.38 (m, 4H), 2.93 (t, J=5.1 Hz, 2H), 2.82 (t, J=5.8 Hz, 2H), 2.67 (dd, J=7.7 Hz, J=7.7 Hz, 2H), 2.53 (br s, 1H), 2.25 (t, J=7.3 Hz, 2H), 2.05 (dt as q, J=6.8 Hz, 6.8 Hz, 2H), 1.94 (quintet, J=7.8 Hz, 2H), 1.40-1.20 (m, 6H), 0.88 (t, J=6.7 Hz, 3H). ¹³C NMR (100 MHz, CDCl₃) δ 173.84, 139.24, 138.69, 130.57, 129.25, 129.23, 128.79, 128.71, 128.49, 127.68, 127.46, 126.36, 126.21, 62.57, 42.42, 36.04, 32.10, 31.48, 30.55, 29.28, 27.21, 26.38, 25.73, 25.67, 22.54, 14.03.

### N—((R)-1-Hydroxypropan-2-yl)-4-(2-((2Z,5Z,8Z)-tetradeca-2,5,8-trien-1-yl)phenyl)butanamide (Compound 4.3.5)

Compound 4.3.5 was synthesized in a similar manner as compound 4.3.4. The resulting crude was chromatographed on silica gel (20-100% EtOAc/hexane) to afford pure title compound in 85% yield as light yellow oil. ¹H NMR (400 MHz, CDCl₃) δ 7.20-7.12 (m, 4H), 5.65-5.30 (m, 7H) [overlapping patterns i.e. 5.56 (d, J=5.3 Hz, 1H), 5.56-5.30 (m, 6H)], 4.13-4.00 (m, 1H), 3.65 (dd, J=10.9 Hz, J=3.4 Hz, 1H), 3.52 (dd, J=10.9 Hz, J=6.0 Hz, 1H), 3.42 (d, J=5.4 Hz, 2H), 2.93 (t, J=5.0 Hz, 2H), 2.83 (t, J=5.8 Hz, 2H), 2.76 (br s, 1H), 2.67 (dd, J=7.7 Hz, J=7.7 Hz, 2H), 2.23 (t, J=7.3 Hz, 2H), 2.05 (dt as q, J=6.8 Hz, 6.8 Hz, 2H), 1.94 (quintet, J=7.8 Hz, 2H), 1.40-1.23 (m, 6H), 1.16 (d, J=6.8 Hz, 3H), 0.88 (t, J=6.7 Hz, 3H). ¹³C NMR (100 MHz, CDCl₃) δ 173.38, 139.25, 138.70, 130.57, 129.28, 129.23, 128.79, 128.72, 128.48, 127.68, 127.46, 126.37, 126.21, 67.37, 47.79, 36.19, 32.10, 31.48, 30.53, 29.28, 27.21, 26.42, 25.73, 25.67, 22.54, 17.03, 14.03.

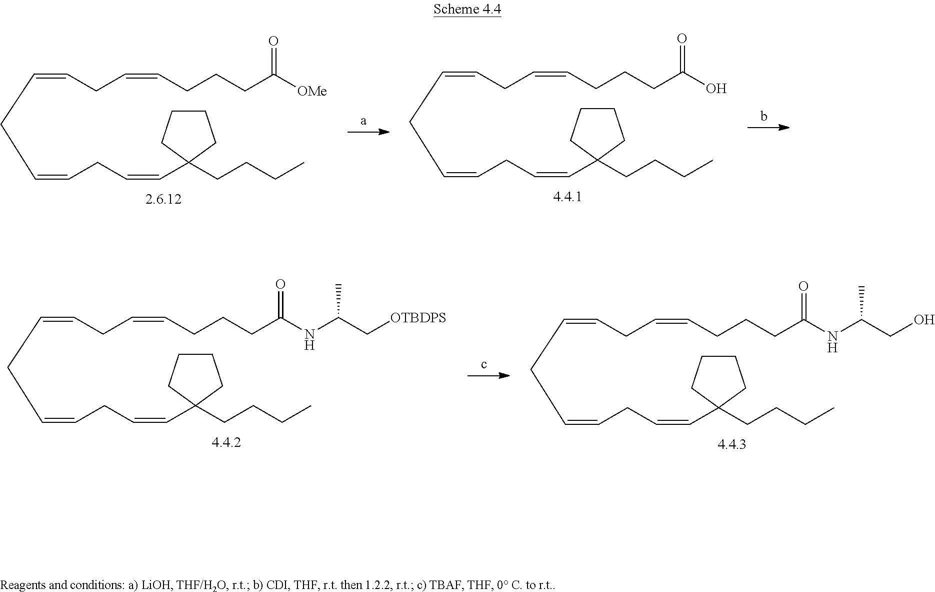

### (5Z,8Z,11Z,14Z)-15-(1-Butylcyclopentyl)pentadeca-5,8,11,14-tetraenoic Acid (Compound 4.4.1)

To a stirred solution of methyl ester 2.6.12 (1 equiv.) in anhydrous THF (0.15 M) at room temperature under an argon atmosphere, was added 1 M aqueous LiOH solution (5 equiv.). Stirring was continued for 24 h, and then the reaction mixture was acidified with 5% HCl to pH 3, and extracted with Et₂O. The combined organic extracts were washed with brine, dried over MgSO₄ and evaporated under reduced pressure. Purification by flash column chromatography on silica gel (5-15% EtOAc/hexane) gave 4.4.1 in 79% yield as a colorless oil. IR (neat): 2928, 2853, 1750 (C═O), 1362, 760, 723 cm⁻¹; ¹³C NMR (100 MHz, CDCl₃) δ 177.4, 139.3, 129.0, 128.7, 128.7, 128.2, 128.1, 127.8, 127.5, 47.9, 40.6, 39.6, 32.9, 27.9, 27.2, 26.4, 25.7, 25.6, 24.5, 24.1, 23.5, 14.1. LC/MS analysis (Waters MicroMass ZQ system) showed retention time 5.8 min for the title compound.

### (5Z,8Z,11Z,14Z)-15-(1-Butylcyclopentyl-N—((R)-1-((tert-butyldiphenylsilyl)oxy)propan-2-yl)pentadeca-5,8,11,14-tetraenamide (Compound 4.4.2)

A solution of (5Z,8Z,11Z,14Z)-15-(1-Butylcyclopentyl)pentadeca-5,8,11,14-tetraenoic acid 4.4.1 (1 equiv.) and dried carbonyldiimidazole (4 equiv.) in anhydrous THF (0.06 M) at room temperature under an argon atmosphere, was stirred for 2 hours and then a solution of (R)-1-((tert-butyldiphenylsilyl)oxy)propan-2-amine 1.2.2 (5 equiv.) in THF was added. The reaction mixture was stirred overnight and then diluted with water and EtOAc. The organic phase was separated and the aqueous phase extracted with EtOAc. The combined organic layer was washed with brine, dried over MgSO₄ and concentrated under reduced pressure. Purification by flash column chromatography on silica gel (20-30% EtOAc/hexane) gave 4.4.2 in 27% yield as a colorless oil. IR (neat): 3301, 3015, 2920, 2864, 1647 (C═O), 1057, 828, 726 cm⁻¹; ¹³C NMR (100 MHz, CDCl₃) δ 173.2, 139.3, 135.3, 133.3, 129.7, 129.1, 129.0, 128.8, 128.7, 128.2, 128.1, 127.8, 127.5, 68.9, 48.0, 47.9, 41.6, 39.6, 36.1, 29.6, 28.0, 27.2, 26.5, 25.7, 25.8, 25.4, 24.1, 23.5, 17.0, 14.1. LC/MS analysis (Waters MicroMass ZQ system) showed retention time 7.4 min for the title compound.

### (5Z,8Z,11Z,14Z)-15-(1-Butylcyclopentyl)-N—((R)-1-hydroxypropan-2-yl)pentadeca-5,8,11,14-tetraenamide (Compound 4.4.3)

To a solution of 4.4.2 (1 equiv.) in dry THF (0.02 M) at 0° C. under an argon atmosphere, was added TBAF 1M solution in THF (5 equiv.) dropwise. The reaction mixture was stirred for 10 min at 0° C. and for 2 hours at room temperature, and then it was quenched with saturated aqueous NH₄Cl at 0° C. and extracted with EtOAc. The combined organic extracts were washed with brine, dried over MgSO₄ and the solvent was evaporated under reduced pressure at 35-37° C. The residue was purified by flash column chromatography on silica gel (40% EtOAc/hexane) to give 4.4.3 in 52% yield as a colorless oil. IR (neat): 3301, 2929, 1641 (C═O), 1546, 1452, 1248, 1050, 691 cm⁻¹; ¹H NMR (400 MHz, CDCl₃) δ 5.54 (br s, 1H, >NH), 5.46-5.32 (m, 7H), 5.19 (dt, J=11.6 Hz, J=7.5 Hz, 1H), 4.08 (m, 1H, >CHCH₃), 3.67 (dd, J=10.9 Hz, J=3.5 Hz, 1H, —CH₂OH), 3.53 (dd, J=10.9 Hz, J=6.1 Hz, 1H, —CH₂OH), 2.90 (m as t, J=5.9 Hz, 2H), 2.82 (dt, J=10.8 Hz, J=5.6 Hz, 4H), 2.20 (t, J=7.4 Hz, 2H, 2-H), 2.12 (dd, J=6.8 Hz, J=6.7 Hz, 2H, 4-H), 1.73 (quintet, J=7.5 Hz, 2H, 3-H), 1.70-1.52 (m, 8H), 1.49-1.39 (m, 2H), 1.36-1.20 (m, 4H), 1.17 (d, J=6.8 Hz, 3H, >CHCH₃), 0.88 (t, J=6.9 Hz, 3H, 20-H). ¹³C NMR (100 MHz, CDCl₃) δ 173.6, 139.3, 129.0, 128.8, 128.7, 128.2, 128.1, 127.8, 127.5, 67.5, 47.9, 47.8, 40.6, 39.6, 36.0, 29.6, 28.0, 27.2, 26.6, 25.7, 25.6, 25.4, 24.1, 23.5, 17.0, 14.1. LC/MS analysis (Waters MicroMass ZQ system) showed retention time 5.7 min for the title compound.

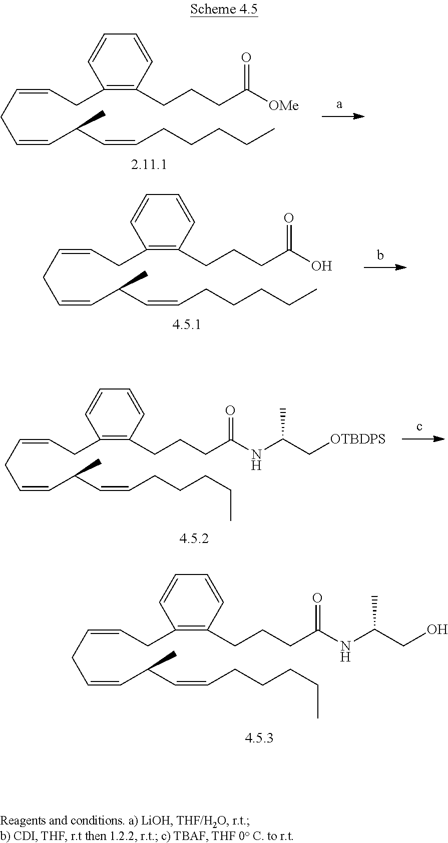

Experimental procedures for the syntheses of all compounds depicted in scheme 4.5 were similar to those described for related syntheses in scheme 4.3.

### N—((R)-1-hydroxypropan-2-yl)-4-(2-((S,2Z,5Z,8Z)-7-methyltetradeca-2,5,8-trien-1-yl)phenyl)butanamide (Compound 4.5.3)

The synthesis of this compound was achieved with a method similar to the one used for the synthesis of compound 4.3.5. The final compound was isolated as a colorless oil. ¹H NMR (500 MHz, CDCl₃) δ 7.15 (s, 4H), 5.56-5.44 (m, 2H), 5.35-5.20 (m, 4H), 4.12-4.05 (m, 1H), 3.71-3.63 (m, 1H), 3.59-3.51 (m, 1H), 3.49 (ddq, J=8.1, 7.5, 6.5 Hz, 1H), 3.42 (d, J=5.4 Hz, 2H), 3.02-2.88 (m, 2H), 2.66 (t, J=8.1 Hz, 2H), 2.38 (t, J=7.4 Hz, 2H), 2.10-2.01 (m, 2H), 1.99-1.87 (m, 2H), 1.34 (d, J=6.8 Hz, 2H), 1.28 (m, 4H), 1.17 (d, J=6.7 Hz, 3H), 1.02 (d, J=6.7 Hz, 3H), 0.87 (t, J=6.8 Hz, 3H).

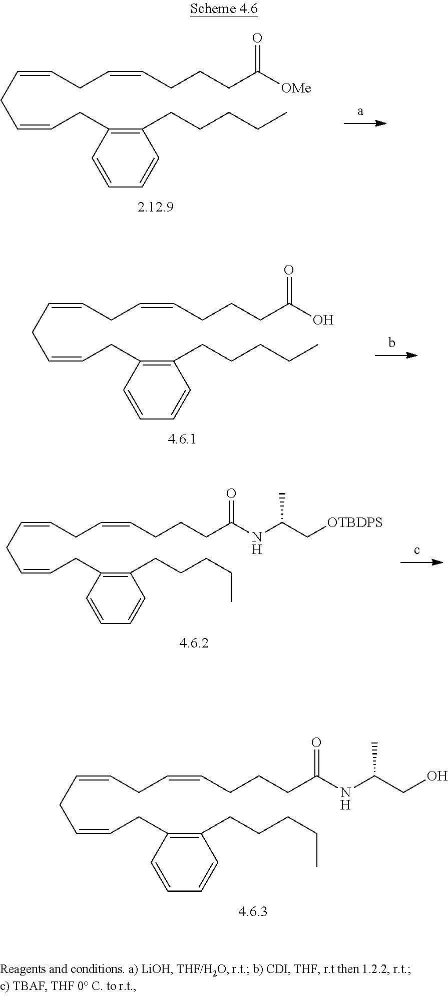

Experimental procedures for the syntheses of all compounds depicted in scheme 4.6 were similar to those described for related syntheses in scheme 4.3

### (5Z,8Z,11Z)—N—((R)-1-hydroxypropan-2-yl)-13-(2-pentylphenyl)trideca-5,8,11-trienamide (Compound 4.6.3)

The synthesis of this compound was achieved with a method similar to the one used for the synthesis of compound 4.3.5. The final compound was isolated as a colorless oil. ¹H NMR (500 MHz, CDCl₃) S 7.19-7.13 (m, 4H), 5.58-5.35 (m, 7H), 4.08 (ddq, J=7.3, J=7.1, J=6.3 Hz, 1H), 3.67 (dd, J=10.6, J=2.7 Hz, 1H), 3.54 (dd, J=10.8, J=6.0 Hz, 1H), 3.44 (d, J=6.6 Hz, 2H), 2.95 (t, J=6.5 Hz, 2H), 2.84 (t, J=6.5 Hz, 2H), 2.63 (t, J=8.0 Hz, 2H), 2.20 (t, J=7.5 Hz, 2H), 2.14 (q, J=6.9 Hz, 2H), 1.74 (quintet, J=7.4 Hz, 2H), 1.60 (quintet, J=7.5 Hz, 2H), 1.42-1.34 (m, 4H), 1.17 (d, J=6.9 Hz, 3H), 0.92 (t, J=7.2 Hz, 3H).

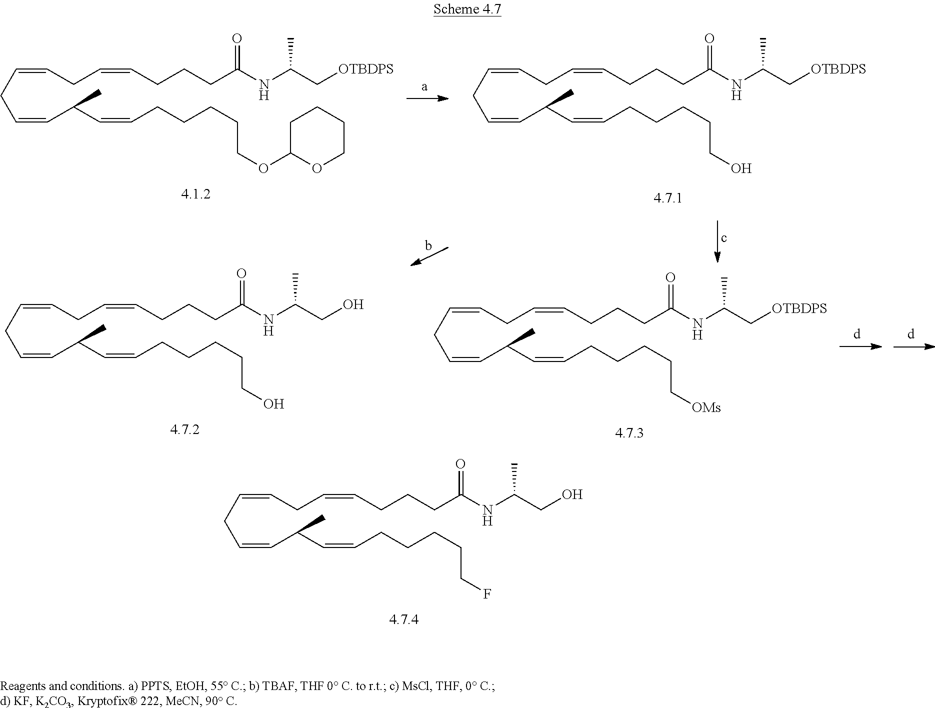

Experimental procedures for the synthesis of all compounds depicted in scheme 4.7 were similar to those described in related syntheses in scheme 4.1.

### (S,5Z,8Z,11Z,14Z)-20-Fluoro-N—((R)-1-hydroxypropan-2-yl)-13-methylicosa-5,8,11,14-tetraenamide (Compound 4.7.4)

To a stirred solution of (S,6Z,9Z,12Z,15Z)-20-(((R)-1-hydroxypropan-2-yl)amino)-8-methyl-20-oxoicosa-6,9,12,15-tetraen-1-yl methanesulfonate (1 equiv.) in dry MeCN (0.01 M) at r.t was added under argon, KF (5 equiv.), K₂CO₃ (2 equiv.) and Kryptofix® 222 (5 mg). The reaction mixture was heated to 90° C. for 15 mins. The reaction mixture was then diluted with EtOAc and washed with water and brine. The organic layer was dried over MgSO₄ and the solvent was evaporated under reduced pressure. The resulting residue was chromatographed on silica gel (80% EtOAc in hexanes) to afford tittle compound in 55% yield as a yellow oil). Mass spectrum (ESI) m/z (relative intensity) 400 (28), 395 (29), 394 (M⁺+H, 100) Exact mass (ESI) calculated for C₂₄H₄₁NO₂F (M⁺+H), 394.3121. found, 394.3117.

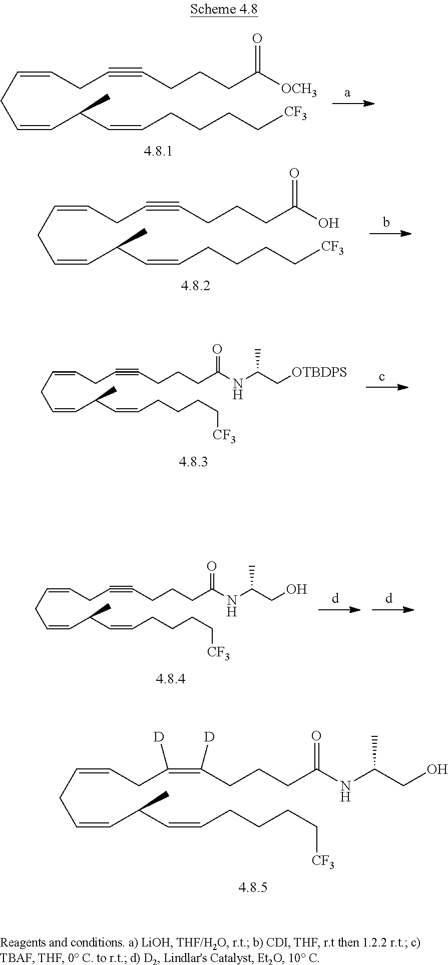

The starting methyl ester 4.8.1 was synthesized by following methods described for similar compounds (e.g., schemes 2.8, 2.9). Experimental procedures for the synthesis of all compounds depicted in scheme 4.8 were similar to those described in related syntheses in scheme 4.1.

### (S,5Z,8Z,11Z,14Z)-20,20,20-Trifluoro-N—((R)-1-hydroxypropan-2-yl)-13-methylicosa-5,8,11,14-tetraenamide (Compound 4.8.5)

To a stirred solution of alkyne 4.8.4 (1 equiv.) in dry Et₂O was added under argon Lindlar's Catalyst (110% w/w) and quinoline (20% w/w). The atmosphere was then swapped to a D₂ one and the reaction mixture was stirred at 10° C. for 1 h. The reaction mixture was filtered through a pad of Celite and the filtrate was evaporated under reduced pressure. The resulting residue was chromatographed (80% EtOAc in hexanes) to afford title compound.

### 5. Chemoenzymatic Synthesis of Glycerides

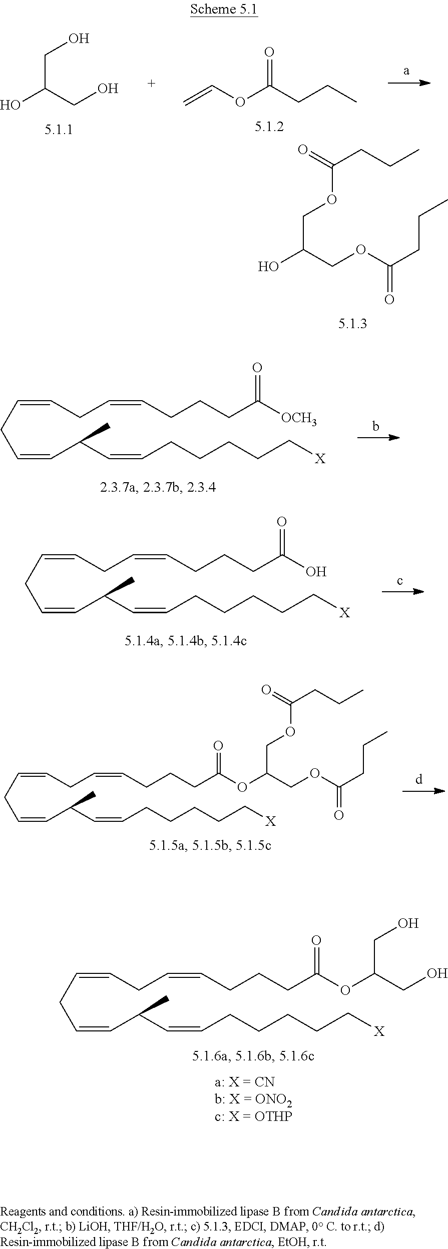

### (S,5Z,8Z,11Z,14Z)-20-Cyano-13-methylicosa-5,8,11,14-tetraenoic Acid (Compound 5.1.4)

To a stirred solution of methyl (S,5Z,8Z,11Z,14Z)-20-cyano-13-methylicosa-5,8,11,14-tetraenoate (1 equiv.) in THF (0.05 M) at room temperature under an argon atmosphere was added lithium hydroxide (5 equiv., 1 M in water). The reaction mixture was stirred at the same temperature overnight. the reaction mixture was acidified with 5% HCl to pH 3, and extracted with Et₂O. The combined organic extracts were washed with brine, dried over MgSO₄ and evaporated under reduced pressure. The resulting residue was chromatographed on silica gel (20-40% ethyl acetate-hexanes) to afford title carboxylic acid in 91% yield as a colorless oil. ¹H NMR (500 MHz, CDCl₃) δ 5.46-5.31 (m, 3H), 5.32-5.18 (m, 4H), 3.50-3.37 (m, 1H), 2.90-2.72 (m, 4H), 2.41-2.29 (m, 4H) [overlapping patterns i.e., 2.33 (t, J=7.5 Hz, 2H), 2.29 (t, J=7.5 Hz, 2H)], 2.18-2.06 (m, 4H), 1.72 (quintet, J=7.4 Hz, 2H), 1.66 (quintet, J=7.5 Hz, 2H), 1.52-1.42 (m, 2H), 1.42-1.33 (m, 2H), 1.01 (d, J=6.8 Hz, 3H). Compounds 5.1.4b and 5.1.4c were synthesized in a similar manner.

### 2-(((S,5Z,8Z,11Z,14Z)-20-Cyano-13-methylicosa-5,8,11,14-tetraenoyl)oxy)propane-1,3-diyl Dibutyrate (Compound 5.1.5a)

To a stirred solution of (S,5Z,8Z,11Z,14Z)-20-Cyano-13-methylicosa-5,8,11,14-tetraenoic acid (1 equiv.) and 2-hydroxypropane-1,3-diyl dibutyrate (1.8 equiv.) in anhydrous CH₂Cl₂ [0.05 M] under an argon atmosphere at 0° C. was added N,N-dimethylaminopyridine (DMAP, 10 equiv.) and N-dimethylaminopropyl-N′-ethylcarbodiimide (EDCI, 5 equiv.). The reaction mixture was allowed to warm to ambient temperature overnight. The reaction mixture was quenched with Sat. NH₄Cl and 5% HCl extracted with Et₂O, washed with brine, dried over MgSO₄, concentrated under reduced pressure, and chromatographed on silica gel, (20-40% Et₂O/hexane) to afford title compound at 86% yield as a colorless oil. ¹³C NMR (100 MHz, CDCl₃) δ 173.1, 172.6, 134.9, 128.9, 128.9, 128.4, 128.2, 127.4, 125.7, 119.5, 69.0, 62.0, 35.9, 33.6, 30.5, 28.9, 28.3, 27.1, 26.5, 25.8, 25.6, 25.4, 24.8, 22.0, 18.3, 17.1, 15.23, 13.6. Compounds 5.1.5b and 5.1.5c were synthesized in a similar manner.

### 1,3-Dihydroxypropan-2-yl (S,5Z,8Z,11Z,14Z)-20-cyano-13-methylicosa-5,8,11,14-tetraenoate (Compound 5.1.6a)

To a stirred solution of 2-(((S,5Z,8Z,11Z,14Z)-20-cyano-13-methylicosa-5,8,11,14-tetraenoyl)oxy)propane-1,3-diyl dibutyrate (1 equiv.) in anhydrous EtOH [0.02 M] was added of immobilized C. Antarctica lipase B. After 1.5 h, the resin was filtered and washed with an additional four portions of EtOH. The filtrate was concentrated under vacuum and chromatographed on silica gel, (10-40% Acetone/hexanes) to give afford title compound at 51% as a colorless oil. ¹³C NMR (100 MHz, CDCl₃) δ 173.8, 134.9, 134.9, 129.0, 128.9, 128.4, 128.2, 127.4, 125.7, 119.7 75.1, 62.4, 33.7, 30.5, 28.9, 28.6, 27.2, 26.5, 25.9, 25.6, 25.3, 24.8, 22.0, 17.1. Compounds 5.1.6b and 5.1.6c were synthesized in a similar manner.

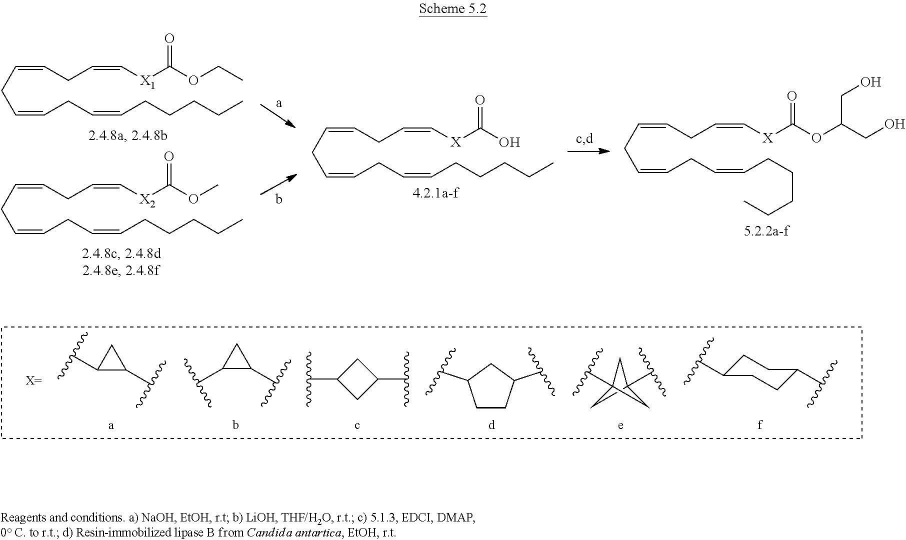

### 1,3-Dihydroxypropan-2-yl 2-((1Z,4Z,7Z,10Z)-hexadeca-1,4,7,10-tetraen-1-yl)cyclopropane-1-carboxylate (Compound 5.2.2a)

To a stirred solution of 2-((1Z,4Z,7Z,10Z)-hexadeca-1,4,7,10-tetraen-1-yl)cyclopropane-1-carboxylic acid (1 equiv.) and 2-hydroxypropane-1,3-diyldibutyrate (1.8 equiv.) in anhydrous CH₂Cl₂ [0.05 M] under an argon atmosphere at 0° C. was added N,N-dimethylaminopyridine (DMAP, 10 equiv.) and N-dimethylaminopropyl-N′-ethylcarbodiimide (EDCI, 5 equiv.). The reaction mixture was allowed to warm to ambient temperature overnight. The reaction mixture was quenched with Sat. NH₄Cl and 5% HCl extracted with Et₂O, washed with brine, dried over MgSO₄, concentrated under reduced pressure. The residue was chromatographed on silica gel (5-20% Et₂O/hexane) to afford protected ester in 92% yield as colorless oil. To a stirred solution of above protected ester (1 equiv.) in anhydrous EtOH [0.02 M] was added of immobilized C. Antarctica lipase B. After 1.5 h, the resin was filtered and washed with an additional four portions of EtOH. The filtrate was concentrated under vacuum and chromatographed on silica gel, (20-70% EtOAc/hexane) to afford title compound in 49% yield as colorless oil. ¹H NMR (500 MHz, CDCl₃) δ 5.47-5.30 (m, 7H), 4.92 (quintet, J=4.7 Hz, 1H), 4.83 (tt, J=11.0 Hz, J=1.5 Hz, 1H), 3.84 (d, J=4.6 Hz, 4H), 3.02-2.90 (m, 2H), 2.85 (t, J=4.8 Hz, 2H), 2.81 (t, J=6.3 Hz, 2H), 2.30-2.22 (m, 3H), 2.05 (q, J=7.0 Hz, 2H), 1.68 (quintet of d, J=4.0 Hz, J=1.1 Hz, 1H), 1.47 (quintet of d, J=4.4 Hz, J=0.5 Hz, 1H), 1.40-1.24 (m, 6H), 0.98 (ddd, J=8.2 Hz, J=6.3 Hz, J=4.3 Hz, 1H), 0.89 (t, J=6.8 Hz, 3H). ¹³C NMR (100 MHz, CDCl₃) δ 173.89, 130.53, 129.75, 129.52, 128.72, 128.71, 127.71, 127.59, 127.49, 75.61, 65.82, 62.55, 31.49, 29.29, 27.19, 26.08, 25.62, 22.54, 21.91, 21.64, 16.70, 15.23, 14.03. Compounds 5.2.2b-f were synthesized in a similar manner.

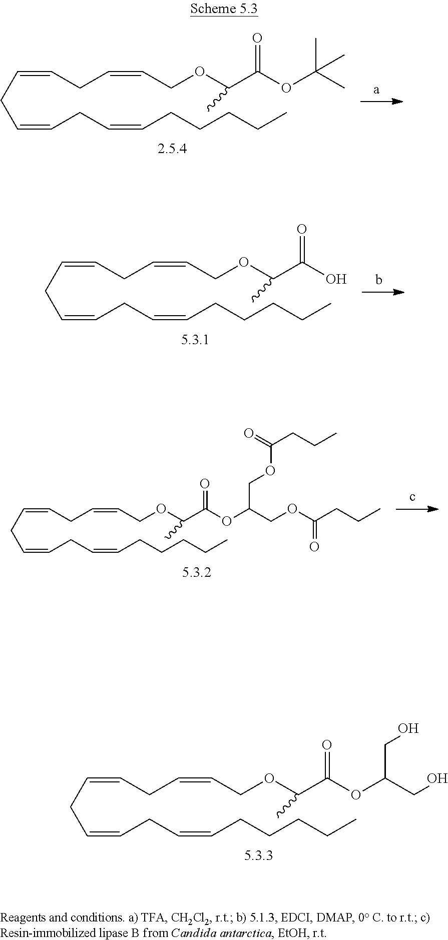

### 2-(((2Z,5Z,8Z,11Z)-Heptadeca-2,5,8,11-tetraen-1-yl)oxy)propanoic Acid (Compound 5.3.1

To a stirred solution of tert-butyl 2-(((2Z,5Z,8Z,11Z)-heptadeca-2,5,8,11-tetraen-1-yl)oxy)propanoate (1 equiv.) in anhydrous CH₂Cl₂ [0.5 M] were added 3 drops of trifluoroacetic acid (TFA). The reaction mixture was stirred at the room temperature for 1 h. The reaction mixture was quenched with Sat. NaHCO₃, and extracted with Et₂O. The combined organic extracts were washed with brine, dried over MgSO₄ and evaporated under reduced pressure. The resulting residue was chromatographed on silica gel (20-40% EtOAc/hexane) to afford title carboxylic acid in 80% yield as colorless oil. ¹H NMR (500 MHz, CDCl₃) δ 11.44 (br s, 1H), 5.69-5.52 (m, 2H), 5.45-5.28 (m, 6H), 4.21 (dd, J=12.1 Hz, J=6.3 Hz, 1H), 4.15 (dd, J=12.1 Hz, J=6.3 Hz, 1H), 4.05 (q, J=6.9 Hz, 1H), 2.88-2.78 (m, 6H), 2.05 (dt, J=7.1 Hz, J=7.1 Hz, 2H), 1.47 (d, J=6.8 Hz, 3H), 1.40-1.24 (m, 6H), 0.89 (t, J=6.8 Hz, 3H).

### 2-((2-(((2Z,5Z,8Z,11Z)-Heptadeca-2,5,8,11-tetraen-1-yl)oxy)propanoyl)oxy)propane-1,3-diyl Dibutyrate (Compound 5.3.2)

Compound 5.3.2 was synthesized in a similar manner to compound 5.1.5a. The resulting crude product was chromatographed on silica gel (5-20% Et₂O/hexane) to afford title compound in 92% yield as colorless oil. ¹H NMR (500 MHz, CDCl₃) δ 5.69-5.52 (m, 2H), 5.45-5.28 (m, 7H), 4.46-4.12 (m, 6H), 4.05 (q, J=6.9 Hz, 1H), 2.88-2.78 (m, 6H), 2.29 (t, J=7.3 Hz, 4H), 2.05 (dt, J=7.1 Hz, J=7.1 Hz, 2H), 1.63 (sextet, J=7.4 Hz, 4H), 1.47 (d, J=6.8 Hz, 3H), 1.40-1.24 (m, 6H), 0.93 (t, J=7.3 Hz, 6H), 0.89 (t, J=6.8 Hz, 3H).

### 1,3-Dihydroxypropan-2-yl 2-(((2Z,5Z,8Z,11Z)-heptadeca-2,5,8,11-tetraen-1-yl)oxy)propanoate (Compound 5.3.3)

Compound 5.3.3 was synthesized in a similar manner to compound 5.1.6a. The resulting crude product was chromatographed on silica gel (20-70% EtOAc/hexane) to afford title compound in 49% yield as colorless oil. ¹H NMR (400 MHz, CDCl₃) δ 5.67-5.52 (m, 2H), 5.45-5.29 (m, 6H), 5.00 (quintet, J=4.8 Hz, 1H), 4.22 (dd, J=12.2 Hz, J=5.1 Hz, 1H), 4.15-4.05 (m, 2H), 3.92-3.83 (m, 4H), 3.61 (br s, 2H), 2.92-2.75 (m, 6H), 2.06 (dt, J=6.7 Hz, J=6.7 Hz, 2H), 1.46 (d, J=6.8 Hz, 3H), 1.42-1.24 (m, 6H), 0.90 (t, J=6.5 Hz, 3H). ¹³C NMR (100 MHz, CDCl₃) δ 173.40, 132.47, 130.56, 128.90, 128.75, 127.63, 127.44, 127.27, 125.51, 75.54, 74.10, 65.57, 62.27, 62.22, 31.48, 29.28, 27.19, 25.89, 25.61, 25.58, 22.53, 18.78, 14.02.

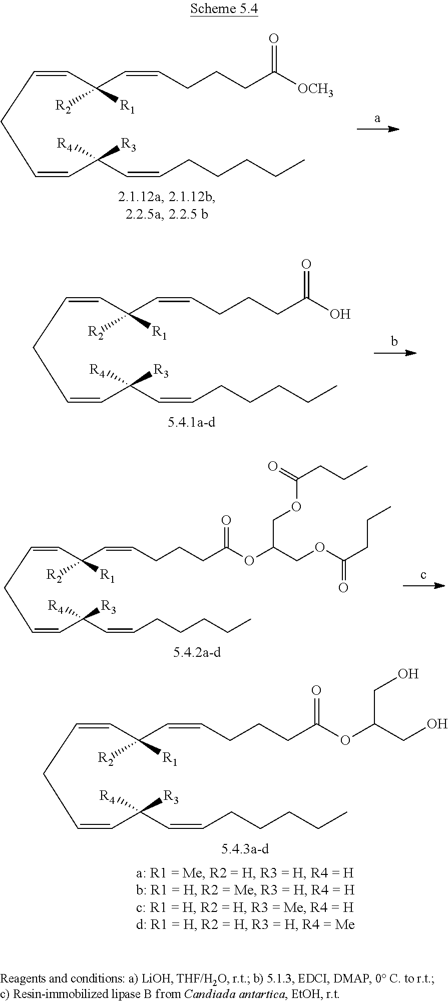

The synthesis of 5.4.1a, 5.4.1b, and 5.4.1d along with the spectroscopic data for the compounds are identical to those we disclosed in Yingpeng, et al. J. Med. Chem. (2018) 61, 8639-8657, the content of which is hereby incorporated by reference.

The synthesis of 5.4.1c along with the spectroscopic data for the compound are identical to those reported in Papahatjis et al. (2010), Chem. Eur. J, 16, 4091-4099 the content of which is hereby incorporated by reference.

### 2-(((R,5Z,8Z,11Z,14Z)-7-Methylicosa-5,8,11,14-tetraenoyl)oxy)propane-1,3-diyl Dibutyrate (Compound 5.4.2a)

Compound 5.4.2a was synthesized in a similar manner to compound 5.1.5a. The crude product obtained after work up was purified by flash column chromatography on silica gel (10-25% Et₂O/hexane) and gave 5.4.2a (95% yield) as a colorless oil. IR (neat): 3013, 2962, 2931, 2857, 1743 (C═O), 1457, 1170, 1096 cm⁻¹; Mass spectrum (ESI) m/z (relative intensity) 550 (71), 533 (M⁺+H, 100), 215 (33). Exact mass (ESI) calculated for C₃₂H₅₃O₆ (M⁺+H), 533.3842. found, 533.3846. LC/MS analysis (Waters MicroMass ZQ system) showed purity 97% and retention time 6.9 min for the title compound.

### 2-(((S,5Z,8Z,11Z,14Z)-7-Methylicosa-5,8,11,14-tetraenoyl)oxy)propane-1,3-diyl Dibutyrate (Compound 5.4.2b)

Compound 5.4.2b was synthesized in a similar manner to compound 5.1.5a. The crude product obtained after work up was purified by flash column chromatography on silica gel (10-25% Et₂O/hexanes) and gave 5.4.2b (92% yield) as a colorless oil. The IR, Mass spectrum (ESI) m/z (relative intensity) 550 (68), 533 (M⁺+H, 100), 445 (21), 301 (15), 215 (29). Exact mass (ESI) calculated for C₃₂H₅₃O₆ (M⁺+H), 533.3842. found, 533.3848. LC/MS analysis (Waters MicroMass ZQ system) showed purity 97% and retention time 6.9 min for the title compound.

### 2-(((S,5Z,8Z,11Z,14Z)-13-Methyleicosa-5,8,11,14-tetraenoyl)oxy)propane-1,3-diyldibutyrate (Compound 5.4.2c)

Compound 5.4.2c was synthesized in a similar manner to compound 5.1.5a. The crude product obtained after work up was purified by flash column chromatography on silica gel (10-25% Et₂O/hexane) and gave 5.4.2c (95%) as a colorless oil. IR (neat): 3009, 2962, 2928, 2857, 1743 (C═O), 1457, 1169, 1095 cm⁻¹ Mass spectrum (EI) m/z (relative intensity) 532 (M⁺, 5), 300 (4), 215 (60), 71 (100). Exact mass (EI) calculated for C₃₂H₅₂O₆ (M⁺), 532.3764. found, 532.3761. LC/MS analysis (Waters MicroMass ZQ system) showed purity 97% and retention time 6.9 min for the title compound.

### 2-(((R,5Z,8Z,11Z,14Z)-13-Methyleicosa-5,8,11,14-tetraenoyl)oxy)propane-1,3-diyldibutyrate (Compound 5.4.2d)

Compound 5.4.2d was synthesized in a similar manner to compound 5.1.5a. The crude product obtained after work up was purified by flash column chromatography on silica gel (10-25% Et₂O/hexanes) and gave 5.4.2d (92% yield) as a colorless oil. Mass spectrum (ESI) m/z (relative intensity) 550 (52), 533 (M⁺+H, 100), 445 (15), 301 (11), 215 (29). Exact mass (ESI) calculated for C₃₂H₅₃O₆ (M⁺+H), 533.3842. found, 533.3845. LC/MS analysis (Waters MicroMass ZQ system) showed purity 97% and retention time 6.9 min for the title compound.

### 1,3-Dihydroxypropan-2-yl (R,5Z,8Z,11Z,14Z)-7-methylicosa-5,8,11,14-tetraenoate (Compound 5.4.3a)

Compound 5.4.3a was synthesized in a similar manner to compound 5.1.6a. The crude product obtained after work up was purified by flash column chromatography on silica gel (20-80% EtOAc/hex) and gave 5.4.3a (51% yield) as a colorless oil. IR (neat): 3397, 3013, 2957, 2858, 1733 (C═O), 1458, 1378, 1176, 1036 cm¹; ¹³C NMR (100 MHz, CDCl₃) δ 173.7 (C═O), 135.5 (C═C), 134.6 (C═C), 130.5 (C═C), 128.6 (C═C), 128.0 (C═C), 127.5 (C═C), 126.6 (C═C), 125.9 (C═C), 75.1 (—OCH(CH₂)₂—), 62.5 (—CH₂OH), 33.7, 31.5, 30.5, 29.3, 27.2, 26.8, 25.8, 25.6, 24.8, 22.5, 22.0, 14.0. Mass spectrum (ESI) m/z (relative intensity) 394 (M⁺+2H, 28), 393 (M⁺+H, 100), 375 (16), 301 (18). Exact mass (ESI) calculated for C₂₄H₄₁O₄ (M⁺+H), 393.3005. found, 393.2997. LC/MS analysis (Waters MicroMass ZQ system) showed purity 98% and retention time 5.5 min for the title compound.

### 1,3-Dihydroxypropan-2-yl (S,5Z,8Z,11Z,14Z)-7-methylicosa-5,8,11,14-tetraenoate (Compound 5.4.3b)

Compound 5.4.3b was synthesized in a similar manner to compound 5.1.6a. The crude product obtained after work up was purified by flash column chromatography on silica gel (20-80% EtOAc/hexane) and gave 5.4.3b (52% yield) as a colorless oil. Mass spectrum (ESI) m/z (relative intensity) 394 (M⁺+2H, 28), 393 (M⁺+H, 100), 375 (16), 301 (18). Exact mass (ESI) calculated for C₂₄H₄₁O₄ (M⁺+H), 393.3005. found, 393.3015. LC/MS analysis (Waters MicroMass ZQ system) showed purity 96% and retention time 5.5 min for the title compound.

### 1,3-Dihydroxypropan-2-yl (S,5Z,8Z,11Z,14Z)-13-methylicosa-5,8,11,14-tetraenoate (Compound 5.4.3c)

Compound 5.4.3c was synthesized in a similar manner to compound 5.1.6a. The filtrate was concentrated under vacuum and chromatographed (silica gel, 20-80% EtOAc/hexanes) to give 5.4.3c (53% yield) as a colorless oil. IR (neat): 3399, 3013, 2955, 2857, 1733 (C═O), 1458, 1374, 1176, 1036 cm⁻¹; ¹H NMR (500 MHz, CDCl₃) δ 5.46-5.32 (m, 4H, 5-H, 6-H, 8-H, 9-H), 5.31-5.20 (m, 4H, 11-H, 12-H, 14-H, 15-H), 4.94 (quintet, J=4.7 Hz, 1H, sn-2), 3.84 (t, J=5.3 Hz, 4H, sn-1 and sn-3), 3.45 (sextet, J=7.0 Hz, 1H, 13-H), 2.92-2.77 (m, 4H, 7-H, 10-H), 2.40 (t, J=7.6 Hz, 2H, 2-H), 2.14 (dt, J=7.0 Hz, J=7.0 Hz, 2H, 4-H), 2.03-2.09 (m, 2H, 16-H), 1.97 (t, J=6.1 Hz, 2H, —OH), 1.74 (quintet, J=7.4 Hz, 2H, 3-H), 1.39-1.24 (m, 6H, 17-H, 18-H, 19-H), 1.02 (d, J=6.8 Hz, 3H, >CH—CH₃), 0.89 (t, J=6.8 Hz, 3H, 20-H). ¹³C NMR (100 MHz, CDCl₃) δ 173.7 (C═O), 135.1 (C═C), 134.1 (C═C), 129.0 (C═C), 128.7 (C═C), 128.5 (C═C), 128.2 (C═C), 128.0 (C═C), 125.6 (C═C), 75.1 (—OCH(CH₂)₂—), 62.5 (—CH₂OH), 33.6, 31.5, 30.5, 29.4, 27.5, 26.4, 25.8, 25.5, 24.7, 22.5, 22.0, 14.0. Mass spectrum (EI) m/z (relative intensity) 392 (M⁺, 2), 294 (4), 217 (7), 133 (19), 122 (75), 105 (100), 93 (84). Exact mass (EI) calculated for C₂₄H₄₀O₄ (M⁺), 392.2927. found, 392.2932. LC/MS analysis (Waters MicroMass ZQ system) showed purity 97% and retention time 5.5 min for the title compound.

### 1,3-Dihydroxypropan-2-yl (R,5Z,8Z,11Z,14Z)-13-methylicosa-5,8,11,14-tetraenoate (Compound 5.4.3d)

Compound 5.4.3d was synthesized in a similar manner to compound 5.1.6a. The crude product obtained after work up was purified by flash column chromatography on silica gel (20-80% EtOAc/hexane) and gave 5.4.3d (49% yield) as a colorless oil: The IR, ¹H NMR (500 MHz, CDCl₃) and ¹³C NMR (100 MHz, CDCl₃) spectrum was identical to that of the enantiomer of 5.4.3c. Mass spectrum (EI) m/z (relative intensity) 392 (M⁺, 3), 133 (23), 105 (90). Exact mass (EI) calculated for C₂₄H₄₀O₄ (M⁺), 392.2927. found, 392.2930. LC/MS analysis (Waters MicroMass ZQ system) showed purity 97% and retention time 5.5 min for the title compound.

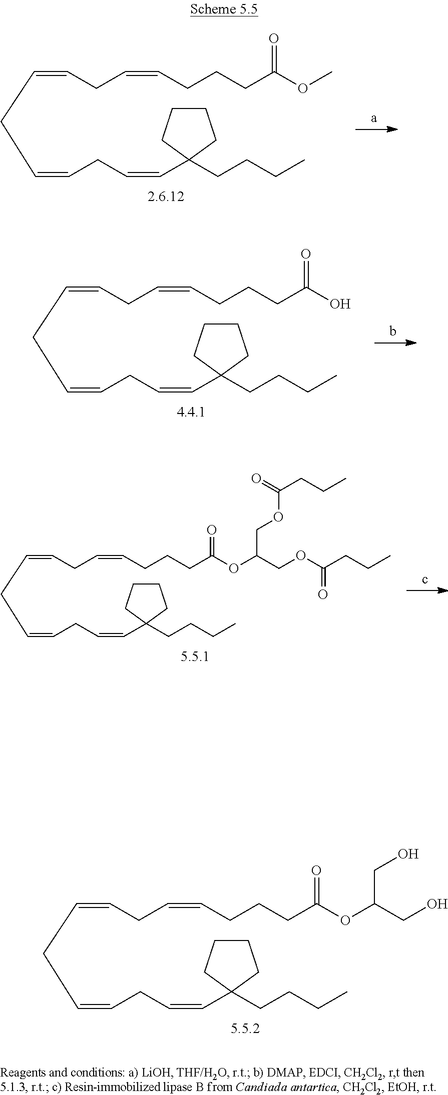 [SMILES: CCCCC1(/C=C\C/C=C\C/C=C\C/C=C\CCCC(=O)OC)CCCC1 ; CCCCC1(/C=C\C/C=C\C/C=C\C/C=C\CCCC(=O)O)CCCC1 ; CCCCC1(/C=C\C/C=C\C/C=C\C/C=C\CCCC(=O)OC(COC(=O)CCC)COC(=O)CCC)CCCC1 ; CCCCC1(/C=C\C/C=C\C/C=C\C/C=C\CCCC(=O)OC(CO)CO)CCCC1]

### 2-(((5Z,8Z,11Z,14Z)-15-(1-Butylcyclopentyl)pentadeca-5,8,11,14-tetraenoyl)oxy)propane-1,3-diyl Dibutyrate (Compound 5.5.1)

To a stirred solution of 4.4.1 (1 equiv.) and 2-hydroxypropane-1,3-diyl dibutyrate (5.1.3) (2 equiv.) in anhydrous CH₂Cl₂ (0.07 M) under an argon atmosphere at 0° C. was added N,N-dimethylaminopyridine (10 equiv.) and N-dimethylaminopropyl-N′-ethylcarbodiimide (5 equiv.). The reaction mixture was allowed to warm to r.t. overnight. The reaction mixture was quenched with Sat. NH₄Cl and 5% HCl, extracted with Et₂O, washed with brine, dried over MgSO₄, concentrated under reduced pressure, and chromatographed on silica gel (10-25% Et₂O/hexane) to give 5.5.1 in 73% as a colorless oil. ¹³C NMR (100 MHz, CDCl₃) δ 173.1, 172.6, 139.3, 129.0, 128.8, 128.8, 128.3, 128.1, 127.8, 127.5, 69.0, 62.0, 47.9, 40.7, 39.6, 35.9, 33.6, 28.0, 27.2, 26.5, 25.7, 25.6, 24.7, 24.1, 23.6, 18.3, 14.1, 13.6.

### 1,3-Dihydroxypropan-2-yl (5Z,8Z,11Z,14Z)-15-(1-butylcyclopentyl)pentadeca-5,8,11,14-tetraenoate (Compound 5.5.2)

To a stirred solution of 2-(((5Z,8Z,11Z,14Z)-15-(1-butylcyclopentyl)pentadeca-5,8,11,14-tetraenoyl)oxy)propane-1,3-diyl dibutyrate (5.5.1) (1 equiv.) in anhydrous EtOH (0.05 M) was added immobilized C. Antarctica lipase B. After 2 h the resin was filtered and washed with an additional four portions of EtOH. The filtrate was concentrated under vacuum and chromatographed on silica gel (20-80% EtOAc/hexanes) to give 5.5.2 in 40% yield as a colorless oil. ¹H NMR (500 MHz, CDCl₃) δ 5.48-5.31 (m, 7H), 5.20 (dt, J=11.6 Hz, J=7.5 Hz, 11H, 14-H), 4.94 (quintet, J=4.7 Hz, 1H), 3.84 (d, J=4.7 Hz, 4H), 2.91 (m as t, J=5.8 Hz, 2H), 2.82 (m, 4H), 2.40 (t, J=7.6 Hz, 2H), 2.14 (dd, J=7.1 Hz, J=7.1 Hz, 2H), 1.74 (quintet, J=7.5 Hz, 2H, 3-H), 1.70-1.52 (m, 6H), 1.50-1.40 (m, 2H), 1.35-1.20 (m, 6H), 0.89 (t, J=7.0 Hz, 3H).

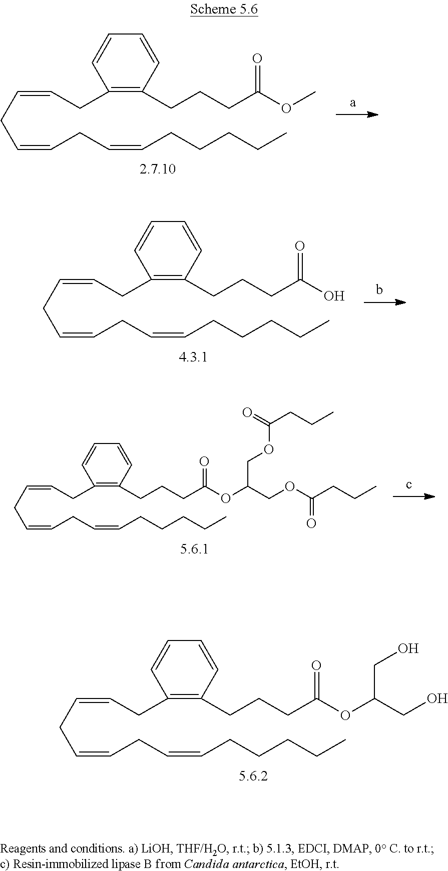 [SMILES: CCCCC/C=C\C/C=C\C/C=C\Cc1ccccc1CCCC(=O)OC ; CCCCC/C=C\C/C=C\C/C=C\Cc1ccccc1CCCC(=O)O ; CCCCC/C=C\C/C=C\C/C=C\Cc1ccccc1CCCC(=O)OC(COC(=O)CCC)COC(=O)CCC ; CCCCC/C=C\C/C=C\C/C=C\Cc1ccccc1CCCC(=O)OC(CO)CO]

### 2-((4-(2-((2Z,5Z,8Z)-Tetradeca-2,5,8-trien-1-yl)phenyl)butanoyl)oxy)propane-1,3-diyl Dibutyrate (Compound 5.6.1)

Compound 5.6.1 was synthesized in a similar manner to compound 5.1.5a. The resulting crude product was chromatographed on silica gel (5-20% Et₂O/hexane) to afford title compound in 92% yield as colorless oil. ¹H NMR (400 MHz, CDCl₃) δ 7.20-7.12 (m, 4H), 5.55-5.25 (m, 7H), 4.31 (dd, J=11.9 Hz, J=4.3 Hz, 2H), 4.15 (dd, J=11.9 Hz, J=5.9 Hz, 2H), 3.48-3.38 (m, 2H), 2.94 (dd, J=5.4 Hz, J=5.3 Hz, 2H), 2.83 (dd, J=5.8 Hz, J=5.8 Hz, 2H), 2.67 (dd, J=7.9 Hz, J=7.6 Hz, 2H), 2.39 (t, J=7.3 Hz, 2H), 2.29 (t, J=7.3 Hz, 4H), 2.05 (dt as q, J=6.9 Hz, J=6.9 Hz, 2H), 1.94 (quintet, J=7.6 Hz, 2H), 1.63 (sextet, J=7.4 Hz, 4H), 1.40-1.23 (m, 6H), 0.93 (t, J=7.3 Hz, 6H), 0.88 (t, J=6.7 Hz, 3H).

### 1,3-Dihydroxypropan-2-yl 4-(2-((2Z,5Z,8Z)-tetradeca-2,5,8-trien-1-yl)phenylbutanoate (Compound 5.6.2)

Compound 5.6.2 was synthesized in a similar manner to compound 5.1.6a. The resulting crude product was chromatographed on silica gel (20-70% EtOAc/hexane) to afford title compound in 49% yield as colorless oil. ¹H NMR (400 MHz, CDCl₃) δ 7.20-7.12 (m, 4H), 5.55-5.30 (m, 6H), 4.92 (quintet, J=4.6 Hz, 1H), 3.83 (d, J=4.6 Hz, 4H), 3.59 (br s, 2H), 3.48-3.38 (m, 2H), 2.94 (dd, J=5.3 Hz, J=5.2 Hz, 2H), 2.83 (dd, J=5.8 Hz, J=5.8 Hz, 2H), 2.68 (dd, J=7.9 Hz, J=6.3 Hz, 2H), 2.44 (t, J=7.3 Hz, 2H), 2.05 (dt as q, J=6.9 Hz, J=6.9 Hz, 2H), 1.94 (quintet, J=7.6 Hz, 2H), 1.40-1.23 (m, 6H), 0.88 (t, J=6.7 Hz, 3H). ¹³C NMR (100 MHz, CDCl₃) δ 173.54, 139.07, 138.69, 130.57, 129.31, 129.26, 128.80, 128.76, 128.45, 127.64, 127.46, 126.45, 126.25, 75.09, 62.44, 62.43, 33.89, 32.01, 31.48, 30.51, 29.28, 27.20, 25.78, 25.73, 25.66, 22.54, 14.03.

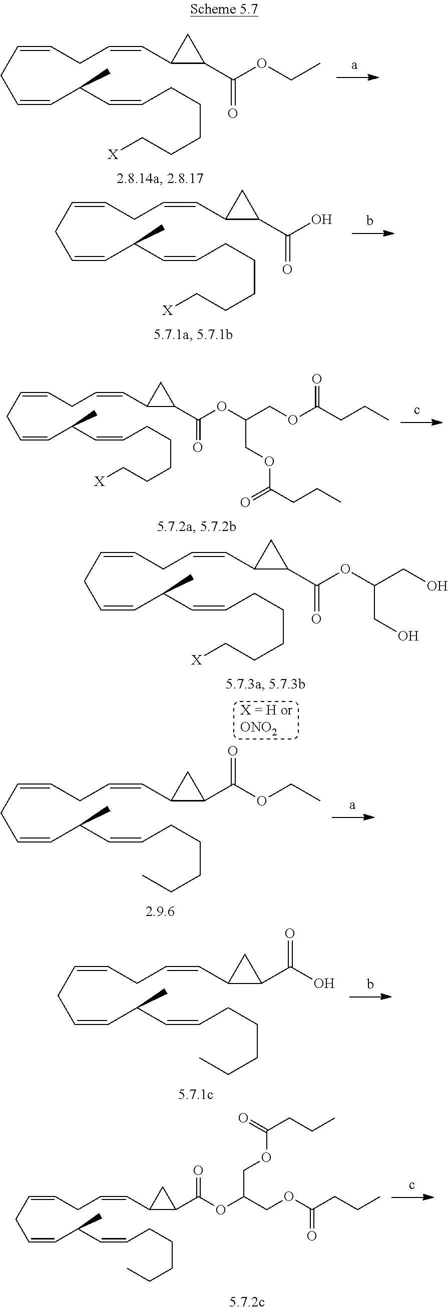

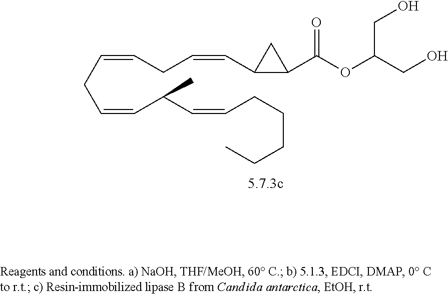 [SMILES: CCCCC/C=C\[C@H](C)/C=C\C/C=C\C/C=C\C1CC1C(=O)OC(CO)CO]

Experimental procedures for the syntheses of all compounds depicted in scheme 5.7 were similar to those described for related syntheses in scheme 5.6

### 1,3-dihydroxypropan-2-yl-2-((S,1Z,4Z,7Z,10Z)-9-methylhexadeca-1,4,7,10-tetraen-1-yl)cyclopropane-1-carboxylate (Compound 5.7.3a)

This compound was synthesized in a manner similar to the one described for 5.6.2. The final compound was isolated as a colorless oil.

### 1,3-dihydroxypropan-2-yl-2-((S,1Z,4Z,7Z,10Z)-9-methyl-16-(nitrooxy)hexadeca-1,4,7,10-tetraen-1-yl)cyclopropane-1-carboxylate (Compound 5.7.3b)

This compound was synthesized in a manner similar to the one described for 5.6.2. The final compound was isolated as a colorless oil. ¹H NMR (500 MHz, CDCl₃) δ 5.47-5.36 (m, 3H), 5.31-5.21 (m, 4H), 4.93 (p, J=4.6 Hz, 1H), 4.84 (t, J=10.2 Hz, 1H), 4.44 (t, J=6.7 Hz, 2H), 3.85 (t, J=5.3 Hz, 5H), 3.44 (ddq, J=7.1, 7.1, 7.0 Hz, 1H), 2.99-2.92 (m, 2H), 2.88-2.79 (m, 2H), 2.22 (ddd, J=6.2, 3.1, 3.1 Hz, 1H), 2.12-2.06 (m, 2H), 2.03-1.97 (m, 2H), 1.76-1.66 (m, 3H), 1.48-1.37 (m and ddd overlapping, 5H, especially 1.45 ddd, J=7.1, 7.1, 7.0), 1.21 (t, J=7.0 Hz, 6H), 1.02 (d, J=6.7 Hz, 3H), 1.00-0.92 (m, 1H).

### 1,3-dihydroxypropan-2-yl 2-((S,1Z,4Z,7Z,10Z)-9-methylhexadeca-1,4,7,10-tetraen-1-yl)cyclopropane-1-carboxylate (Compound 5.7.3c)

This compound was synthesized in a manner similar to the one described for 5.6.2. The final compound was isolated as a colorless oil. ¹H NMR (400 MHz, CDCl₃) δ 5.54 (dt, J=10.7 Hz, J=7.5 Hz, 1H), 5.46-5.22 (m, 7H), 4.95 (quintet, J=4.7 Hz, 1H), 3.86 (d, J=4.7 Hz, 2H), 3.82 (d, J=4.7 Hz, 2H), 3.48 (sextet, J=6.6 Hz, 1H), 3.01-2.81 (m, 4H), 2.17 (quintet, J=8.5 Hz, 1H), 2.11-1.91 (m, 5H), 1.42-1.25 (m, 8H) [overlapping patterns i.e., 1.38 (quintet, J=6.9 Hz, 2H), 1.35-1.25 (m, 6H)], 1.03 (d, J=6.8 Hz, 3H), 0.91 (t, J=7.2 Hz, 3H).

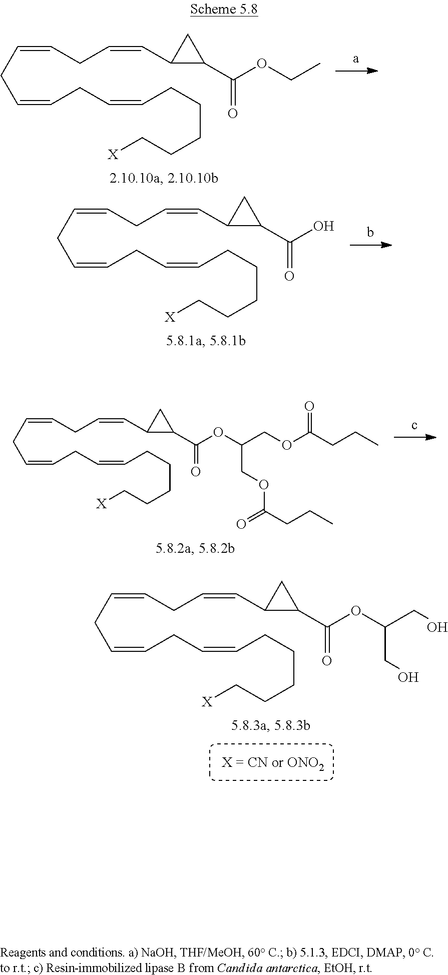

Experimental procedures for the syntheses of all compounds depicted in scheme 5.8 were similar to those described for related syntheses in scheme 5.6

### 1,3-Dihydroxypropan-2-yl-2-((1Z,4Z,7Z,10Z)-16-cyanohexadeca-1,4,7,10-tetraen-1-yl)cyclopropane-1-carboxylate (Compound 5.8.3a)

This compound was synthesized in a manner similar to the one described for 5.6.2. The final compound was isolated as a colorless oil. ¹H NMR (500 MHz, CDCl₃) δ 5.47-5.35 (m, 7H), 4.94 (quintet, J=4.6 Hz, 1H), 4.85 (tt, J=10.6 Hz, J=1.1 Hz, 1H), 3.84 (d, J=4.6 Hz, 4H), 3.02-2.90 (m, 2H), 2.86 (t, J=4.8 Hz, 2H), 2.83 (t, J=4.9 Hz, 2H), 2.35 (t, J=7.1 Hz, 2H), 2.28-2.21 (m, 1H), 2.13-2.02 (m, 4H) [overlapping patterns i.e., 2.11 (q, J=5.8 Hz, 2H), 2.08-2.02 (m, 2H)], 1.72-1.64 (m, 3H) [overlapping patterns i.e., 1.69 (quintet, J=7.5 Hz, 2H), 1.68 (quintet of d, J=4.0 Hz, J=1.1 Hz, 1H)], 1.52-1.35 (m, 5H) [overlapping patterns i.e., 1.48 (quintet of d, J=4.4 Hz, J=0.5 Hz, 1H), 1.45-1.35 (m, 4H)], 0.99 (ddd, J=8.2 Hz, J=6.3 Hz, J=4.3 Hz, 1H).

### 1,3-Dihydroxypropan-2-yl-2-((1Z,4Z,7Z,10Z)-16-(nitrooxy)hexadeca-1,4,7,10-tetraen-1-yl)cyclopropane-1-carboxylate (compound 5.8.3b)

This compound was synthesized in a manner similar to the one described for 5.6.2. The final compound was isolated as a colorless oil. ¹H NMR (500 MHz, CDCl₃) δ 5.47-5.35 (m, 7H), 4.94 (quintet, J=4.7 Hz, 1H), 4.85 (tt, J=10.4 Hz, J=0.6 Hz, 1H), 4.46 (t, J=6.7 Hz, 2H), 3.87 (d, J=4.4 Hz, 4H), 3.00-2.93 (m, 2H), 2.86 (t, J=4.9 Hz, 2H), 2.83 (t, J=4.8 Hz, 2H), 2.27-2.21 (m, 1H), 2.14-1.95 (m, 4H), 1.78-1.68 (m, 3H) [overlapping patterns i.e., 1.75 (quintet, J=7.5 Hz, 2H), 1.71 (quintet of d, J=4.0 Hz, J=1.1 Hz, 1H)], 1.49-1.37 (m, 5H) [overlapping patterns i.e., 1.47 (quintet of d, J=4.4 Hz, J=0.5 Hz, 1H), 1.45-1.37 (m, 4H)], 1.00 (ddd, J=8.2 Hz, J=6.3 Hz, J=4.3 Hz, 1H).

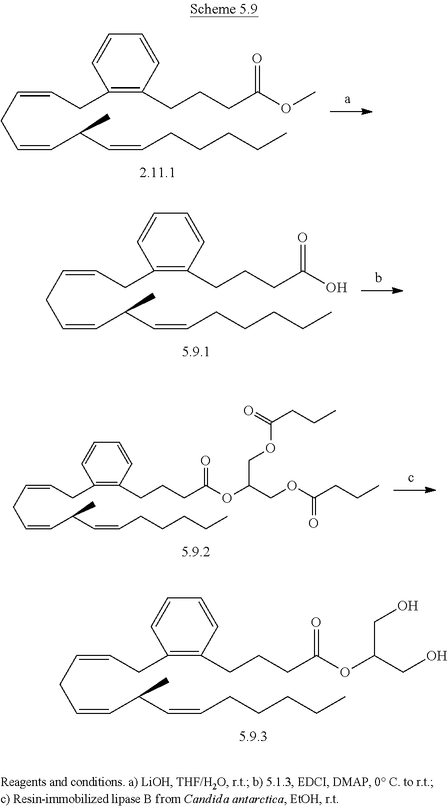 [SMILES: CCCCC/C=C\[C@H](C)/C=C\C/C=C\Cc1ccccc1CCCC(=O)OC ; CCCCC/C=C\[C@H](C)/C=C\C/C=C\Cc1ccccc1CCCC(=O)O ; CCCCC/C=C\[C@H](C)/C=C\C/C=C\Cc1ccccc1CCCC(=O)OC(COC(=O)CCC)COC(=O)CCC ; CCCCC/C=C\[C@H](C)/C=C\C/C=C\Cc1ccccc1CCCC(=O)OC(CO)CO]

Experimental procedures for the syntheses of all compounds depicted in scheme 5.9 were similar to those described for related syntheses in scheme 5.6.

### 1,3-Dihydroxypropan-2-yl 4-(2-((S,2Z,5Z,8Z)-7-methyltetradeca-2,5,8-trien-1-yl)phenylbutanoate (Compound 5.9.3)

This compound was synthesized in a manner similar to the one described for 5.6.2. The final compound was isolated as a colorless oil. ¹H NMR (500 MHz, CDCl₃) δ 7.15 (s, 4H), 5.56-5.44 (m, 2H), 5.35-5.20 (m, 4H), 3.67 (s, 3H), 4.93 (p, J=4.7 Hz, 1H), 3.84 (d, J=4.6 Hz, 4H). 3.49 (ddq, J=8.1, 7.5, 6.5 Hz, 1H), 3.42 (d, J=5.4 Hz, 2H), 3.02-2.88 (m, 2H), 2.66 (t, J=8.1 Hz, 2H), 2.38 (t, J=7.4 Hz, 2H), 2.10-2.01 (m, 2H), 1.99-1.87 (m, 2H), 1.34 (d, J=6.8 Hz, 2H), 1.28 (m, 4H), 1.02 (d, J=6.7 Hz, 3H), 0.87 (t, J=6.8 Hz, 3H).

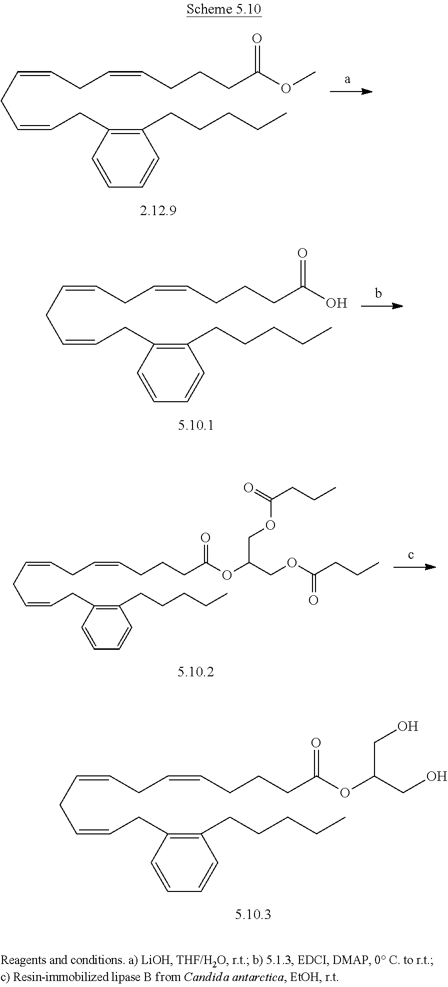 [SMILES: CCCCCc1ccccc1C/C=C\C/C=C\C/C=C\CCCC(=O)OC ; CCCCCc1ccccc1C/C=C\C/C=C\C/C=C\CCCC(=O)O ; CCCCCc1ccccc1C/C=C\C/C=C\C/C=C\CCCC(=O)OC(COC(=O)CCC)COC(=O)CCC ; CCCCCc1ccccc1C/C=C\C/C=C\C/C=C\CCCC(=O)OC(CO)CO]

Experimental procedures for the syntheses of all compounds depicted in scheme 5.10 were similar to those described for related syntheses in scheme 5.6.

### 1,3-Dihydroxypropan-2-yl (R,5Z,8Z,11Z)-13-(2-pentylphenyl)tetradeca-5,8,11-trienoate (Compound 5.10.3)

This compound was synthesized in a manner similar to the one described for 5.6.2. The final compound was isolated as a colorless oil. ¹H NMR (500 MHz, CDCl₃) δ 7.19-7.13 (m, 4H), 5.58-5.35 (m, 6H), 4.94 (quintet, J=4.7 Hz, 1H), 3.84 (d, J=4.1 Hz, 4H), 3.44 (d, J=6.5 Hz, 2H), 2.96 (t, J=6.5 Hz, 2H), 2.85 (t, J=6.5 Hz, 2H), 2.63 (t, J=8.0 Hz, 2H), 2.41 (t, J=7.5 Hz, 2H), 2.15 (q, J=6.9 Hz, 2H), 1.75 (quintet, J=7.4 Hz, 2H), 1.60 (quintet, J=7.5 Hz, 2H), 1.42-1.34 (m, 4H), 0.92 (t, J=7.2 Hz, 3H).

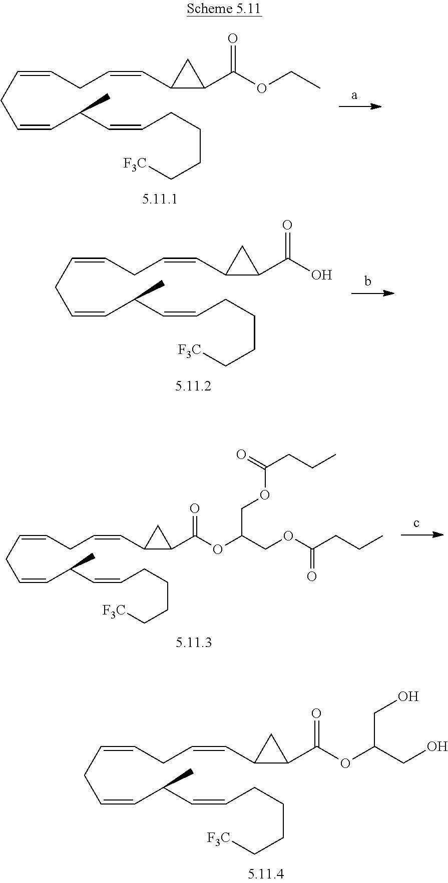 [SMILES: CCOC(=O)C1CC1/C=C\C/C=C\C/C=C\[C@@H](C)/C=C\CCCCC(F)(F)F ; C[C@H](/C=C\C/C=C\C/C=C\C1CC1C(=O)O)/C=C\CCCCC(F)(F)F ; CCCC(=O)OCC(COC(=O)CCC)OC(=O)C1CC1/C=C\C/C=C\C/C=C\[C@@H](C)/C=C\CCCCC(F)(F)F ; C[C@H](/C=C\C/C=C\C/C=C\C1CC1C(=O)OC(CO)CO)/C=C\CCCCC(F)(F)F]

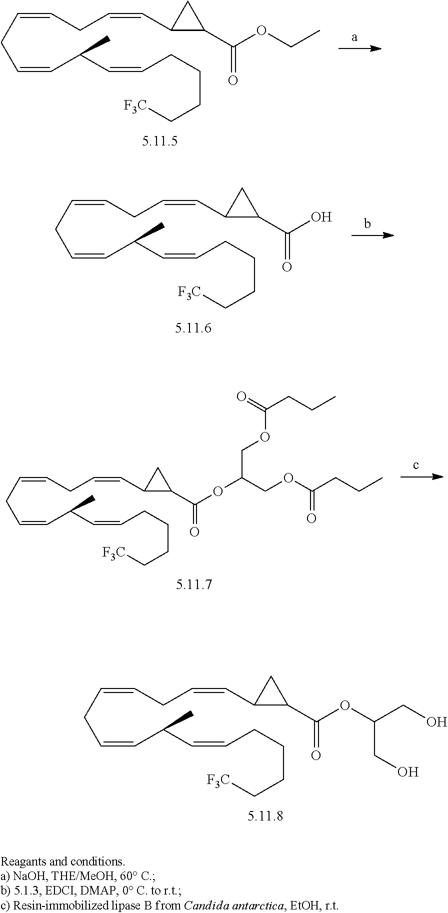 [SMILES: CCOC(=O)C1CC1/C=C\C/C=C\C/C=C\[C@@H](C)/C=C\CCCCC(F)(F)F ; C[C@H](/C=C\C/C=C\C/C=C\C1CC1C(=O)O)/C=C\CCCCC(F)(F)F ; CCCC(=O)OCC(COC(=O)CCC)OC(=O)C1CC1/C=C\C/C=C\C/C=C\[C@@H](C)/C=C\CCCCC(F)(F)F ; C[C@H](/C=C\C/C=C\C/C=C\C1CC1C(=O)OC(CO)CO)/C=C\CCCCC(F)(F)F]

The starting ethyl esters 5.11.1 and 5.11.5 were synthesized by following methods described for similar compounds (e.g., schemes 2.8, 2.9). Experimental procedures for the synthesis of all compounds depicted in scheme 5.11 were similar to those described for related syntheses in scheme 5.7.

### 1,3-dihydroxypropan-2-yl 2-((S,1Z,4Z,7Z,10Z)-16,16,16-trifluoro-9-methylhexadeca-1,4,7,10-tetraen-1-yl)cyclopropane-1-carboxylate (Cis-Isomer Compound 5.11.4)

Viscus oil. ¹H NMR (500 MHz, CDCl₃) δ 5.54 (dt, J=10.7 Hz, J=7.5 Hz, 1H), 5.46-5.22 (m, 7H), 4.95 (quintet, J=4.7 Hz, 1H), 3.86 (d, J=4.7 Hz, 2H), 3.82 (d, J=4.7 Hz, 2H), 3.45 (sextet, J=6.6 Hz, 1H), 3.01-2.81 (m, 4H), 2.19-1.99 (m, 8H) [overlapping patterns i.e., 2.17 (quintet, J=8.5 Hz, 1H), 2.14-1.99 (m, 7H)], 1.57 (quintet, J=7.6 Hz, 2H), 1.44 (quintet, J=7.6 Hz, 2H), 1.33-1.25 (m, 2H), 1.03 (d, J=6.8 Hz, 3H).

### 1,3-dihydroxypropan-2-yl 2-((S,1Z,4Z,7Z,10Z)-16,16,16-trifluoro-9-methylhexadeca-1,4,7,10-tetraen-1-yl)cyclopropane-1-carboxylate (Trans-Isomer Compound 5.11.8)

Viscus oil. ¹H NMR (500 MHz, CDCl₃) δ 5.46-5.34 (m, 3H), 5.32-5.22 (m, 4H), 4.92 (quintet, J=4.7 Hz, 1H), 4.84 (tt, J=10.4 Hz, J=0.6 Hz, 1H), 3.85 (d, J=4.4 Hz, 4H), 3.45 (sextet, J=6.6 Hz, 1H), 3.01-2.79 (m, 4H), 2.26-2.19 (m, 1H), 2.14-2.01 (m, 5H), 1.69 (quintet of d, J=4.8 Hz, J=1.1 Hz, 1H), 1.57 (quintet, J=7.6 Hz, 2H), 1.47-1.40 (m, 3H), 1.03 (d, J=6.8 Hz, 3H), 1.00 (ddd, J=8.2 Hz, J=6.3 Hz, J=4.3 Hz, 1H).

Starting from the appropriate chiral starting materials and by following similar synthetic methods the stereoisomers depicted in scheme 5.12 were synthesized.

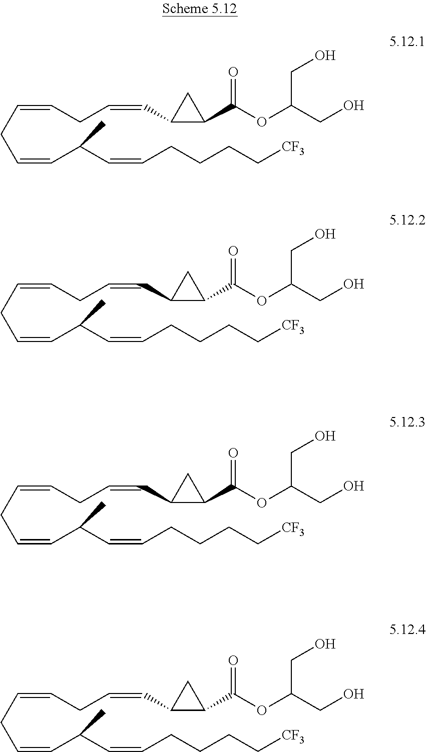 [SMILES: C[C@H](/C=C\C/C=C\C/C=C\[C@@H]1C[C@H]1C(=O)OC(CO)CO)/C=C\CCCCC(F)(F)F ; C[C@H](/C=C\C/C=C\C/C=C\[C@H]1C[C@@H]1C(=O)OC(CO)CO)/C=C\CCCCC(F)(F)F ; C[C@H](/C=C\C/C=C\C/C=C\[C@H]1C[C@H]1C(=O)OC(CO)CO)/C=C\CCCCC(F)(F)F ; C[C@H](/C=C\C/C=C\C/C=C\[C@@H]1C[C@@H]1C(=O)OC(CO)CO)/C=C\CCCCC(F)(F)F]

### 1,3-dihydroxypropan-2-yl (1R,2S)-2-(S,1Z,4Z,7Z,10Z)-16,16,16-trifluoro-9-methylhexadeca-1,4,7,10-tetraen-1-yl)cyclopropane-1-carboxylate (Compound 5.12.1)

Viscus oil. ¹H NMR (500 MHz, CDCl₃) δ 5.46-5.34 (m, 3H), 5.32-5.22 (m, 4H), 4.92 (quintet, J=4.7 Hz, 1H), 4.84 (tt, J=10.4 Hz, J=0.6 Hz, 1H), 3.85 (d, J=4.4 Hz, 4H), 3.45 (sextet, J=6.6 Hz, 1H), 3.01-2.79 (m, 4H), 2.26-2.19 (m, 1H), 2.14-2.01 (m, 5H), 1.69 (quintet of d, J=4.8 Hz, J=1.1 Hz, 1H), 1.57 (quintet, J=7.6 Hz, 2H), 1.47-1.40 (m, 3H), 1.03 (d, J=6.8 Hz, 3H), 1.00 (ddd, J=8.2 Hz, J=6.3 Hz, J=4.3 Hz, 1H).

### 1,3-dihydroxypropan-2-yl (1S,2R)-2-(S,1Z,4Z,7Z,10Z)-16,16,16-trifluoro-9-methylhexadeca-1,4,7,10-tetraen-1-yl)cyclopropane-1-carboxylate (Compound 5.12.2)

Viscus oil. ¹H NMR (500 MHz, CDCl₃) δ 5.46-5.34 (m, 3H), 5.32-5.22 (m, 4H), 4.92 (quintet, J=4.7 Hz, 1H), 4.84 (tt, J=10.4 Hz, J=0.6 Hz, 1H), 3.85 (d, J=4.4 Hz, 4H), 3.45 (sextet, J=6.6 Hz, 1H), 3.01-2.79 (m, 4H), 2.26-2.19 (m, 1H), 2.14-2.01 (m, 5H), 1.69 (quintet of d, J=4.8 Hz, J=1.1 Hz, 1H), 1.57 (quintet, J=7.6 Hz, 2H), 1.47-1.40 (m, 3H), 1.03 (d, J=6.8 Hz, 3H), 1.00 (ddd, J=8.2 Hz, J=6.3 Hz, J=4.3 Hz, 1H).

### 1,3-dihydroxypropan-2-yl (1R,2R)-2-((S,1Z,4Z,7Z,10Z)-16,16,16-trifluoro-9-methylhexadeca-1,4,7,10-tetraen-1-yl)cyclopropane-1-carboxylate (Compound 5.12.3)

Viscus oil. ¹H NMR (500 MHz, CDCl₃) δ 5.54 (dt, J=10.7 Hz, J=7.5 Hz, 1H), 5.46-5.22 (m, 7H), 4.95 (quintet, J=4.7 Hz, 1H), 3.86 (d, J=4.7 Hz, 2H), 3.82 (d, J=4.7 Hz, 2H), 3.45 (sextet, J=6.6 Hz, 1H), 3.01-2.81 (m, 4H), 2.19-1.99 (m, 8H) [overlapping patterns i.e., 2.17 (quintet, J=8.5 Hz, 1H), 2.14-1.99 (m, 7H)], 1.57 (quintet, J=7.6 Hz, 2H), 1.44 (quintet, J=7.6 Hz, 2H), 1.33-1.25 (m, 2H), 1.03 (d, J=6.8 Hz, 3H).

### 1,3-dihydroxypropan-2-yl (1S,2S)-2-((S,1Z,4Z,7Z,10Z)-16,16,16-trifluoro-9-methylhexadeca-1,4,7,10-tetraen-1-yl)cyclopropane-1-carboxylate (Compound 5.12.4)

Viscus oil. ¹H NMR (500 MHz, CDCl₃) δ 5.54 (dt, J=10.7 Hz, J=7.5 Hz, 1H), 5.46-5.22 (m, 7H), 4.95 (quintet, J=4.7 Hz, 1H), 3.86 (d, J=4.7 Hz, 2H), 3.82 (d, J=4.7 Hz, 2H), 3.45 (sextet, J=6.6 Hz, 1H), 3.01-2.81 (m, 4H), 2.19-1.99 (m, 8H) [overlapping patterns i.e., 2.17 (quintet, J=8.5 Hz, 1H), 2.14-1.99 (m, 7H)], 1.57 (quintet, J=7.6 Hz, 2H), 1.44 (quintet, J=7.6 Hz, 2H), 1.33-1.25 (m, 2H), 1.03 (d, J=6.8 Hz, 3H).

### 6. Synthesis of Reverse Amides and Reverse Esters

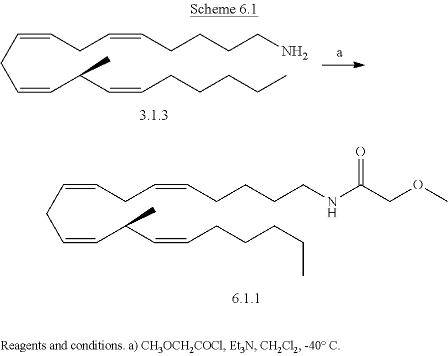 [SMILES: CCCCC/C=C\[C@H](C)/C=C\C/C=C\C/C=C\CCCCN ; CCCCC/C=C\[C@H](C)/C=C\C/C=C\C/C=C\CCCCNC(=O)COC]

### 2-Methoxy-N—((S,5Z,8Z,11Z,14Z)-13-methylicosa-5,8,11,14-tetraen-1-yl)acetamide (Compound 6.1.1)

To a stirred solution of (S,5Z,8Z,11Z,14Z)-13-methylicosa-5,8,11,14-tetraen-1-amine (1 equiv.) in anhydrous CH₂Cl₂ [0.15 M] under an argon atmosphere at −40° C. was added Et₃N (2 equiv.) and methoxyacetyl chloride (1.3 equiv.). The reaction mixture was stirred for 1 hour, quenched with Sat. NH₄Cl, extracted with EtOAc, washed with brine, dried over MgSO₄, concentrated under reduced pressure, and chromatographed on silica gel, (5-20% EtOAc/hexane) to afford title compound at 97% yield as colorless oil. ¹H NMR (500 MHz, CDCl₃) δ 6.50 (br s, 1H), 5.42-5.20 (m, 8H), 3.88 (s, 2H), 3.46 (sextet, J=6.9 Hz, 1H), 3.41 (s, 3H), 3.29 (dt, J=6.9 Hz, J=6.9 Hz, 2H), 2.90-2.77 (m, 2H), 2.14-2.00 (m, 4H), 1.94 (quintet, J=7.0 Hz, 2H), 1.40-1.23 (m, 10H), 1.01 (d, J=6.7 Hz, 3H), 0.88 (t, J=6.6 Hz, 3H). ¹³C NMR (125 MHz, CDCl₃) δ 166.55, 135.04, 134.16, 129.61, 128.32, 128.28, 128.22, 128.20, 125.66, 71.99, 59.14, 38.69, 31.57, 30.50, 29.46, 29.22, 27.51, 26.88, 26.81, 25.84, 25.62, 22.59, 22.03, 14.07.

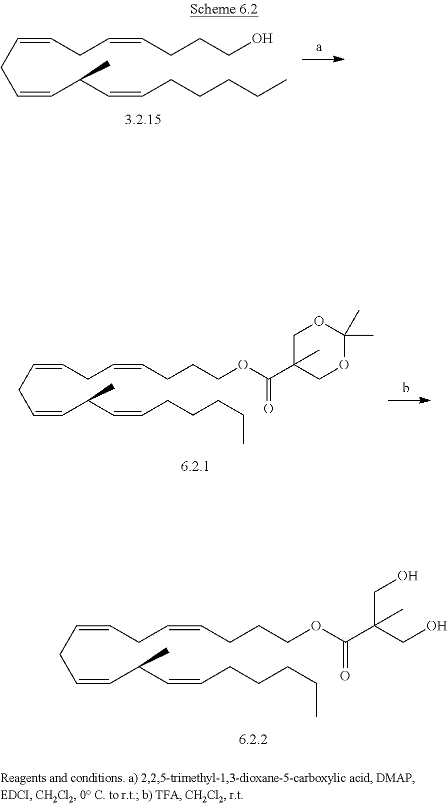

### (S,4Z,7Z,10Z,13Z)-12-Methylnonadeca-4,7,10,13-tetraen-1-yl 2,2,5-trimethyl-1,3-dioxane-5-carboxylate (Compound 6.2.1)

To a stirred solution of 2,2,5-trimethyl-1,3-dioxane-5-carboxylic acid (1 equiv.) and (S,4Z,7Z,10Z,13Z)-12-methylnonadeca-4,7,10,13-tetraen-1-ol (1.3 equiv.) in anhydrous CH₂Cl₂ [0.05 M] under an argon atmosphere at 0° C. was added N,N-dimethylaminopyridine (DMAP, 6 equiv.) and N-dimethylaminopropyl-N′-ethylcarbodiimide (EDCI, 4 equiv.). The reaction mixture was brought to room temperature and stirred overnight. The reaction mixture was quenched with Sat. NH₄Cl, extracted with EtOAc, washed with brine, dried over MgSO₄, concentrated under reduced pressure, and chromatographed on silica gel, (5-20% EtOAc/hexane) to afford title compound at 91% yield as colorless oil. ¹H NMR (500 MHz, CDCl₃) δ 5.44-5.19 (m, 8H), 4.18 (d, J=11.7 Hz, 2H), 4.15 (septet, J=6.5 Hz, 2H), 3.63 (d, J=11.7 Hz, 2H), 3.46 (sextet, J=7.2 Hz, 1H), 2.90-2.75 (m, 4H), 2.19-2.12 (dt, J=6.8 Hz, J=6.8 Hz, 2H), 2.09-2.00 (m, 2H), 1.72 (quintet, J=6.7 Hz, 2H), 1.42 (s, 3H), 1.38 (s, 3H), 1.36-1.24 (m, 6H), 1.20 (s, 3H), 1.01 (d, J=6.7 Hz, 3H), 0.88 (t, J=6.6 Hz, 3H).

### (S,4Z,7Z,10Z,13Z)-12-Methylnonadeca-4,7,10,13-tetraen-1-yl-3-hydroxy-2(hydroxymethyl)-2-methylpropanoate (Compound 6.2.2)

To a stirred solution of (S,4Z,7Z,10Z,13Z)-12-methylnonadeca-4,7,10,13-tetraen-1-yl 2,2,5-trimethyl-1,3-dioxane-5-carboxylate (1 equiv.) in anhydrous CH₂Cl₂ [0.5 M] were added 3 drops of trifluoroacetic acid (TFA). The reaction mixture was stirred at the room temperature for 30 min. The reaction mixture was quenched with Sat. NaHCO₃, and extracted with Et₂O. The combined organic extracts were washed with brine, dried over MgSO₄ and evaporated under reduced pressure. The resulting residue was chromatographed on silica gel (20-40% EtOAc/hexane) to afford title carboxylic acid in 93% yield as colorless oil. ¹H NMR (500 MHz, CDCl₃) δ 5.45-5.19 (m, 8H), 4.18 (t, J=6.5 Hz, 2H), 3.91 (d, J=11.2 Hz, 2H), 3.71 (d, J=11.2 Hz, 2H), 3.46 (sextet, J=7.1 Hz, 1H), 2.90-2.75 (m, 4H), 2.20-2.13 (dt, J=6.7 Hz, J=6.7 Hz, 2H), 2.09-2.00 (m, 2H), 1.72 (quintet, J=6.8 Hz, 2H), 1.39-1.23 (m, 6H), 1.05 (s, 3H), 1.01 (d, J=6.7 Hz, 3H), 0.88 (t, J=6.6 Hz, 3H). ¹³C NMR (125 MHz, CDCl₃) δ 175.97, 135.11, 134.13, 129.05, 128.53, 128.51, 128.24, 127.97, 125.57, 68.74, 68.73, 64.56, 49.04, 31.57, 30.49, 29.45, 28.48, 27.50, 25.82, 25.53, 23.54, 22.58, 22.03, 17.11, 14.07.

1. A compound of formula I or a pharmaceutically acceptable salt thereof, including all stereoisomers and enantiomers: 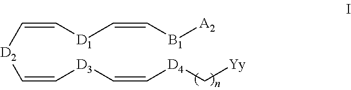
wherein:
A₁ is selected from 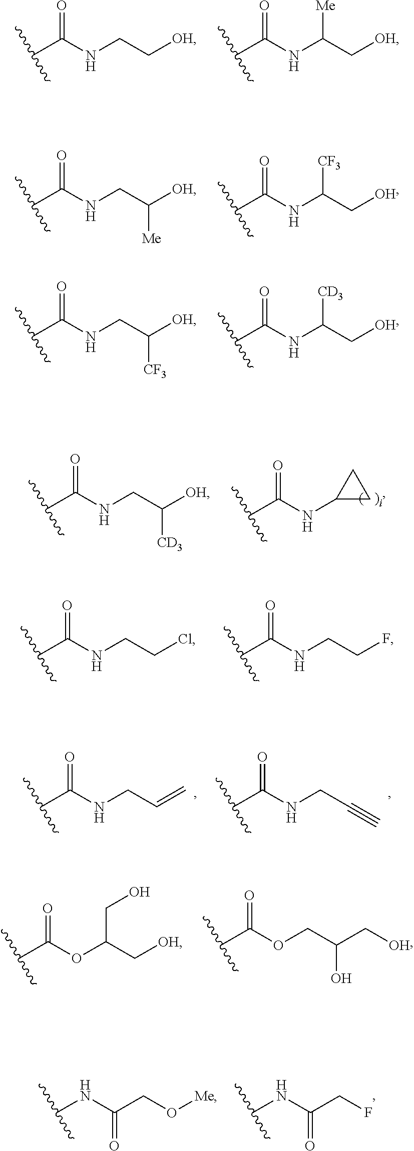 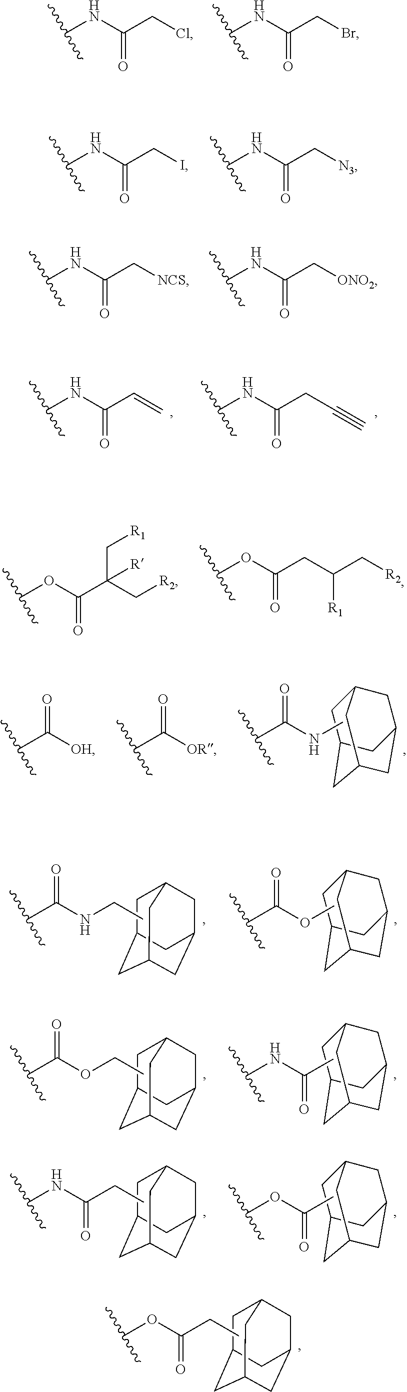
i is an integer from 1 to about 5,
R₁ and R₂ are independently selected from —H, -D, —Cl, —Br, —F, —OH, -Me, —CHF₂, —CH₂F, —CF₃,
R′ is selected from —H, -D and —CH₃,
R″ is -alkyl,
B₁ is selected from 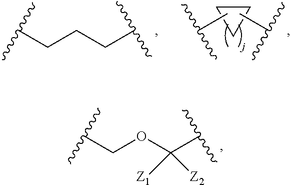
j is an integer from 1 to about 4,
Z₁, Z₂, independently selected from —H, -D, —F, -Me, —CF₃,
D₁, D₂, D₃ and D₄ are each independently selected from —CH₂—, —CF₂—, —CD₂- and 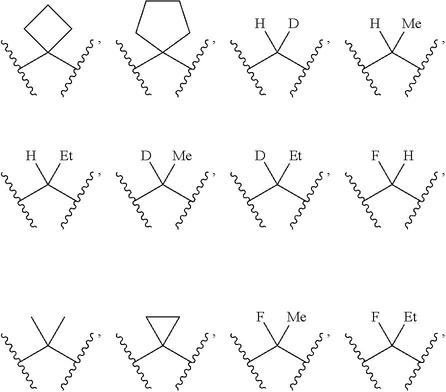
provided that at least one of D₁ or D₂ or D₃ or D₄ in general formula I is a chiral group selected from 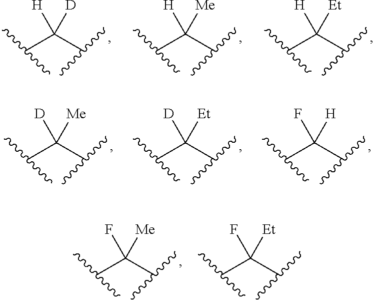
n is an integer from 1 to out 7
Y₁ is selected from -D, —CD₃, —F, —I, —Cl, —Br, —CF₃, —CN, —N₀₂, —SO₂F, —ONO₂, —OH, —SH, —NH₂, —(NH)-Alkyl, —N(Alkyl)₂, -heterocyclic ring, -carbocyclic ring, -Ph, —SPh, —SOPh, —SO₂Ph and 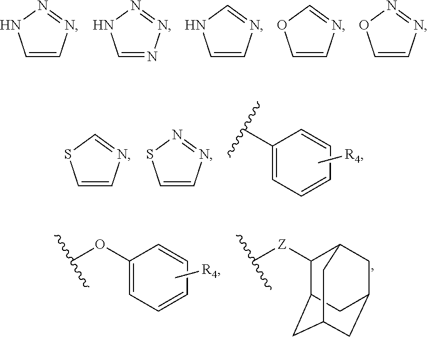
unless otherwise specifically denoted, each of the aforementioned groups is connected to the main structure from any possible position given that it is fulfilling the valency requirements,
R₄ is —F, —CN or —CF₃,
Z is —NH—, —CH₂NH—, —NHCONH—, —COO—, —COOCH₂— or —CH₂—,
wherein the compound cannot be methyl (5Z,8Z,11Z,14Z)-20-hydroxy-13-methylicosa-5,8,11,14-tetraenoate.

2. The compound of claim 1, wherein the compound is selected from the group consisting of:

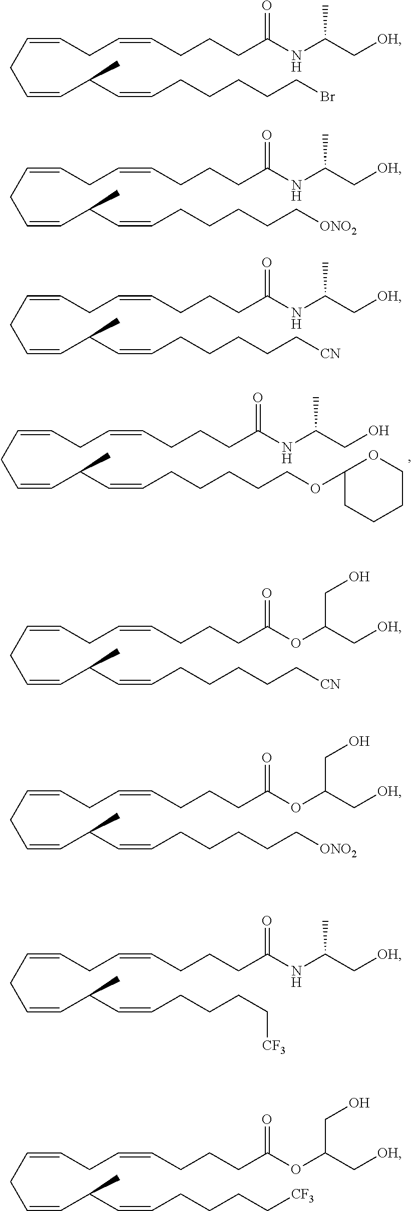

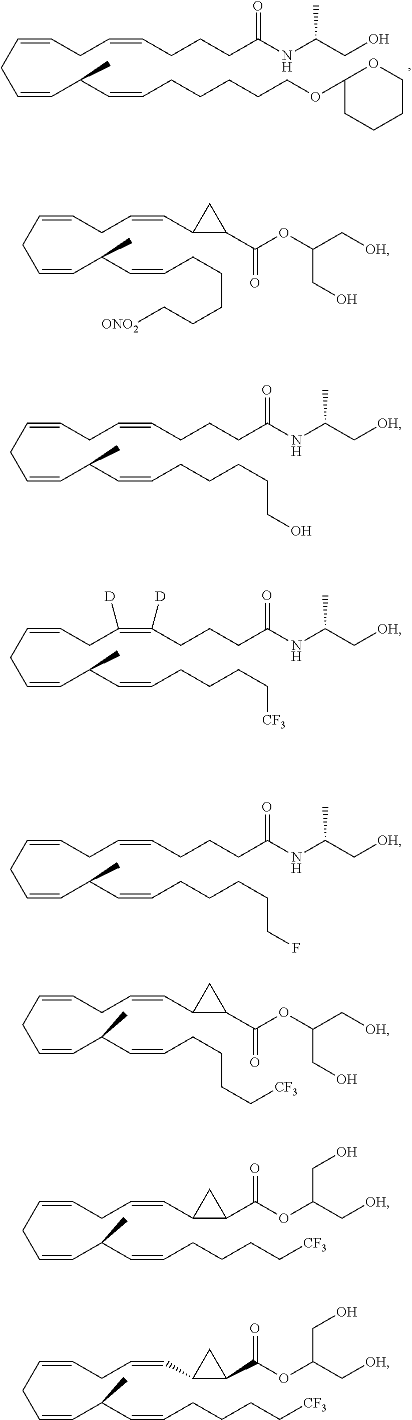

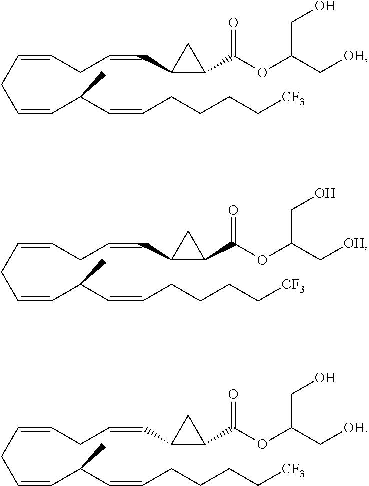 [SMILES: C[C@H](/C=C\C/C=C\C/C=C\[C@H]1C[C@@H]1C(=O)OC(CO)CO)/C=C\CCCCC(F)(F)F ; C[C@H](/C=C\C/C=C\C/C=C\[C@H]1C[C@H]1C(=O)OC(CO)CO)/C=C\CCCCC(F)(F)F ; C[C@H](/C=C\C/C=C\C/C=C\[C@@H]1C[C@@H]1C(=O)OC(CO)CO)/C=C\CCCCC(F)(F)F]

3. A compound of formula II or a pharmaceutically acceptable salt thereof, including all stereoisomers and enantiomers: 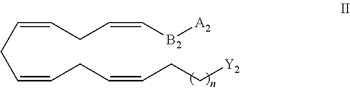
wherein:
A₂ is selected from 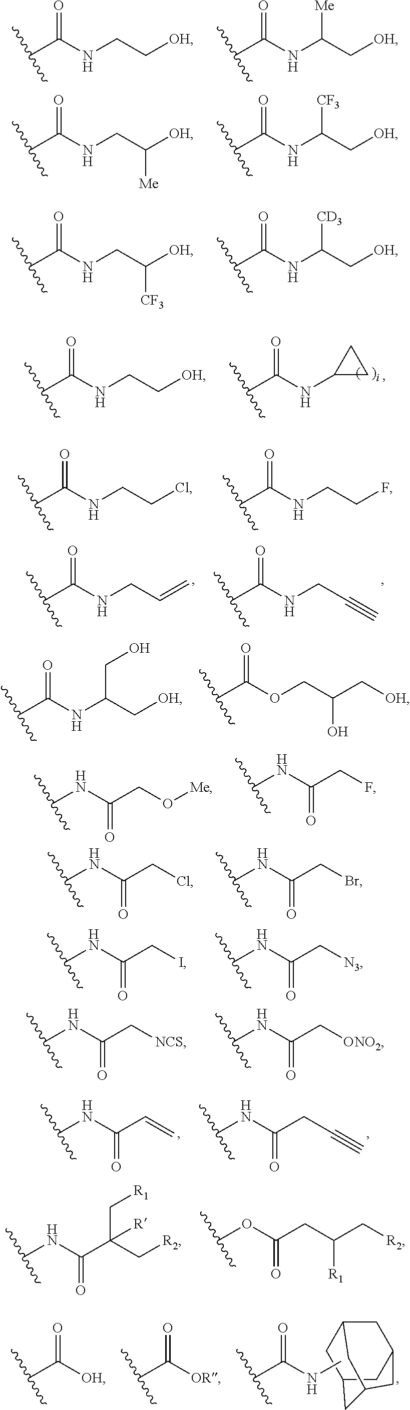 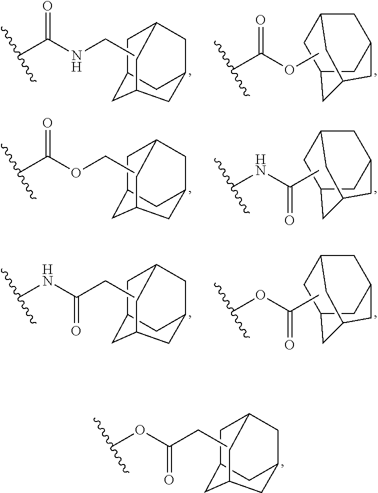
i is an integer from 1 to about 5,
R₁ and R₂ are independently selected from —H, -D, —Cl, —Br, —F, —OH, -Me, —CHF₂, —CH₂F, —CF₃,
R′ is selected from —H, -D and —CH₃,
R″ is -alkyl,
B₂ is selected from 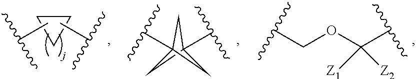
j is an integer from 1 to about 4,
Z₁, Z₂, independently selected from -D, —F, -Me, —CF₃,
n is an integer from 1 to about 7,
Y₂ is selected from -Alkyl, —H, -D, —F, —I, —Cl, —Br, —CF₃, —CN, —NCS, —N₃—NO₂, —SO₂F, —ONO₂, —OH, —SH, —NH₂, —(NH)-Alkyl, —N(Alkyl)₂, -heterocyclic ring, -carbocyclic ring, -Ph, —OPh, —SPh, —SOPh, —SO₂Ph and 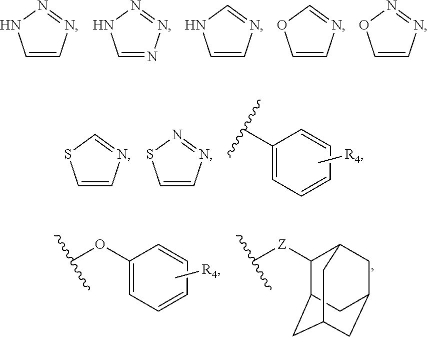
unless otherwise specifically denoted, each of the aforementioned groups is connected to the main structure from any possible position given that it is fulfilling the valency requirements,
R₄ is selected from —F, —CN, —CF₃,
Z is —NH—, —CH₂NH—, —NHCONH—, —COO—, —COOCH₂— or —CH₂—.

4. The compound of claim 3, wherein the compound is selected from the group consisting of:

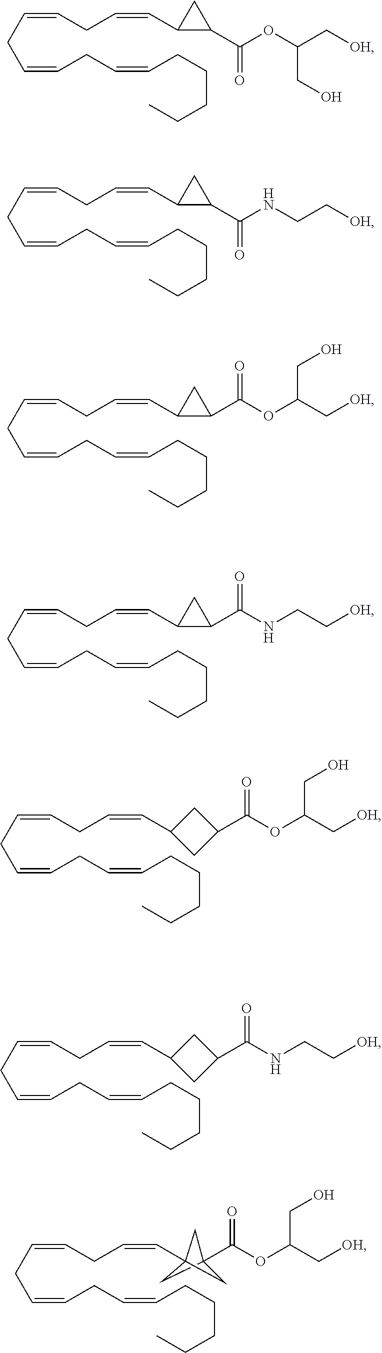

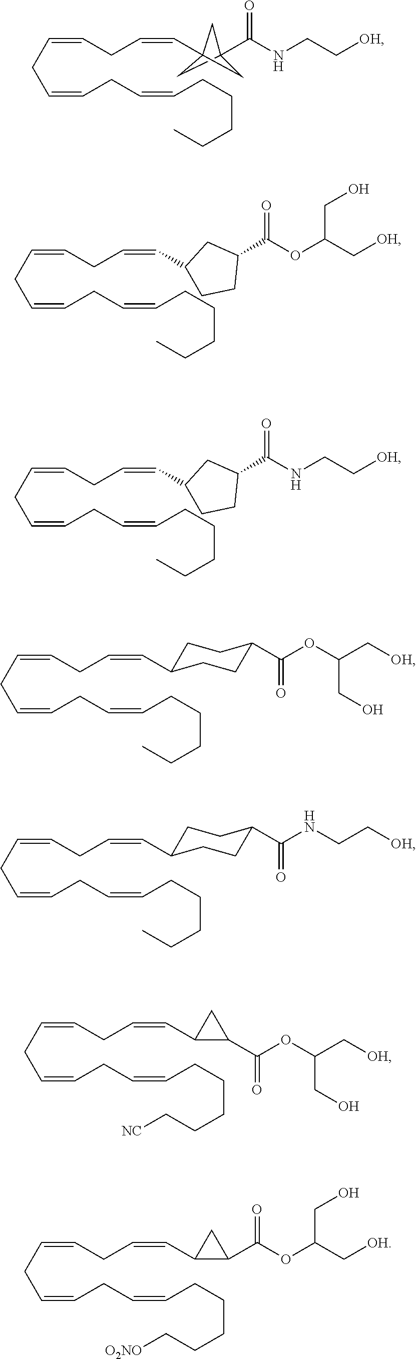

5. A compound of formula III or a pharmaceutically acceptable salt thereof, including all stereoisomers and enantiomers: 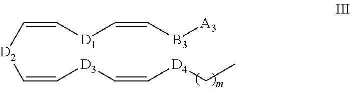
wherein:
A₃ is selected from 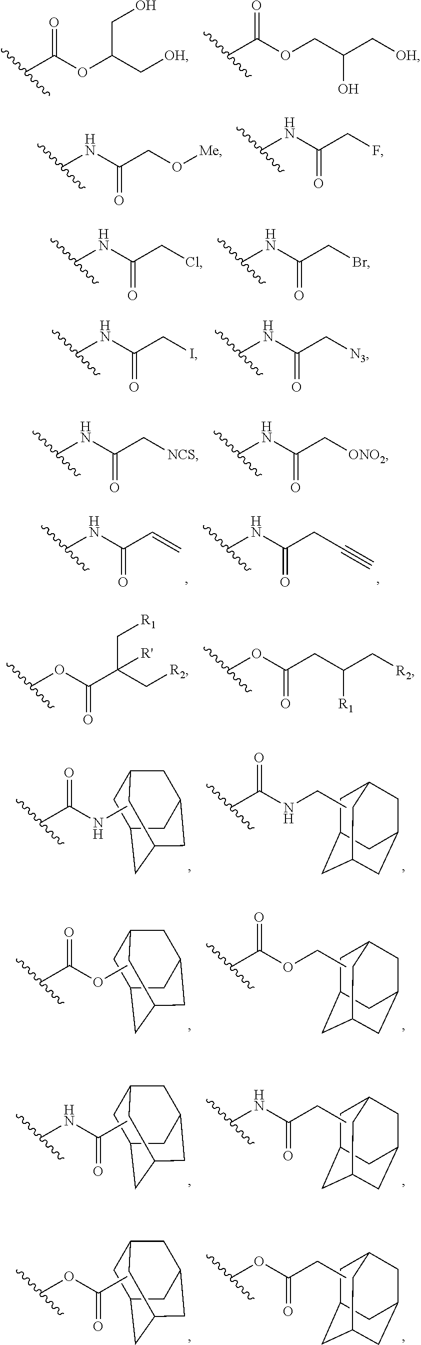
R₁ and R₂ are independently selected from —H, -D, —Cl, —Br, —F, —OH, -Me, —CHF₂, —CH₂F, —CF₃,
R′ is selected from —H, -D and —CH₃,
B₃ is selected from 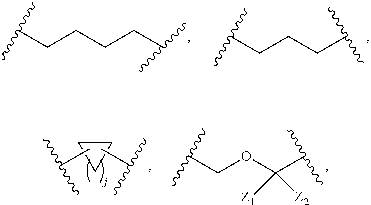
j is an integer from 1 to about 4,
Z₁, Z₂, independently selected from —H, -D, —F, -Me, —CF₃,
D₁, D₂, D₃ and D₄ are each independently selected from —CH₂—, —CF₂—, —CD₂- and 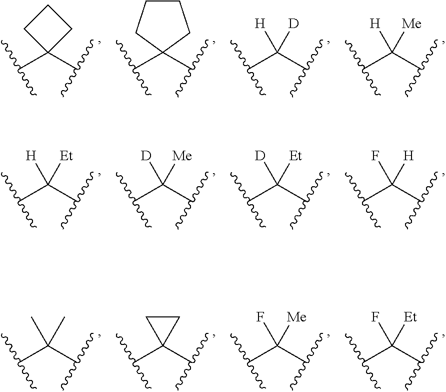
provided that at least one of D₁ or D₂ or D₃ or D₄ in general formula I is a chiral group selected from 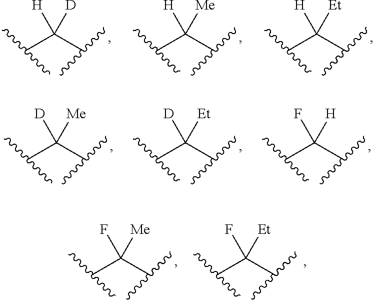
m is an integer from 0 to about 6.

6. The compound of claim 5, wherein the compound is selected from the group consisting of:

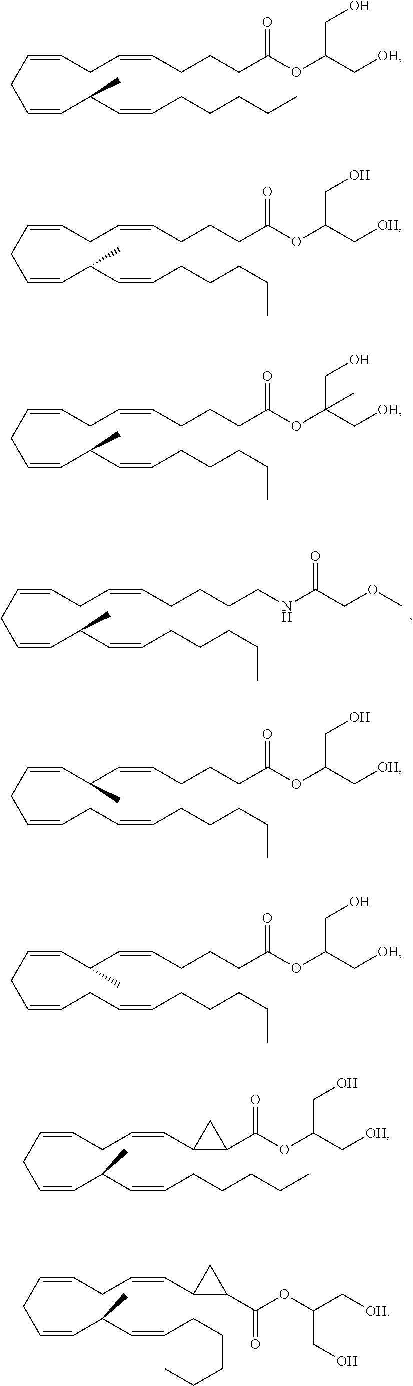

7. A compound of formula IV or a pharmaceutically acceptable salt thereof, including all stereoisomers and enantiomers: 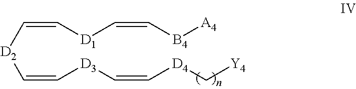
wherein:
A₄ is selected from 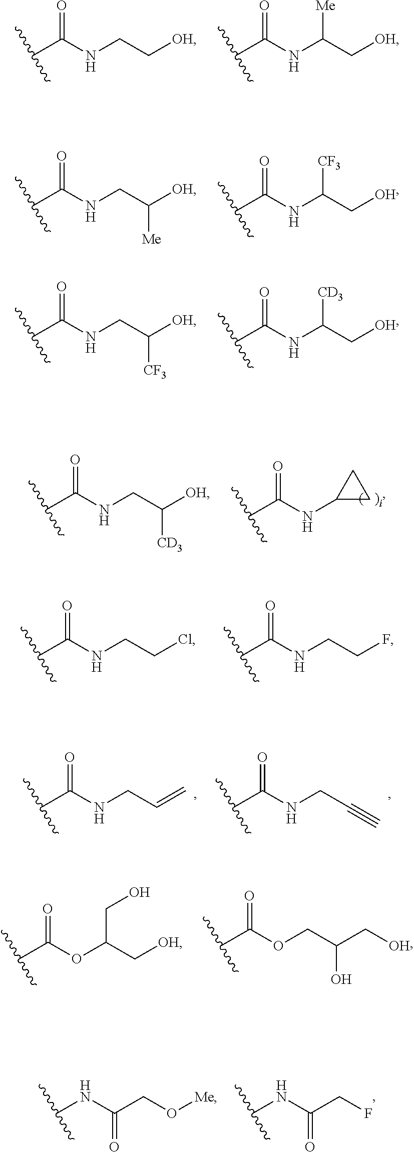 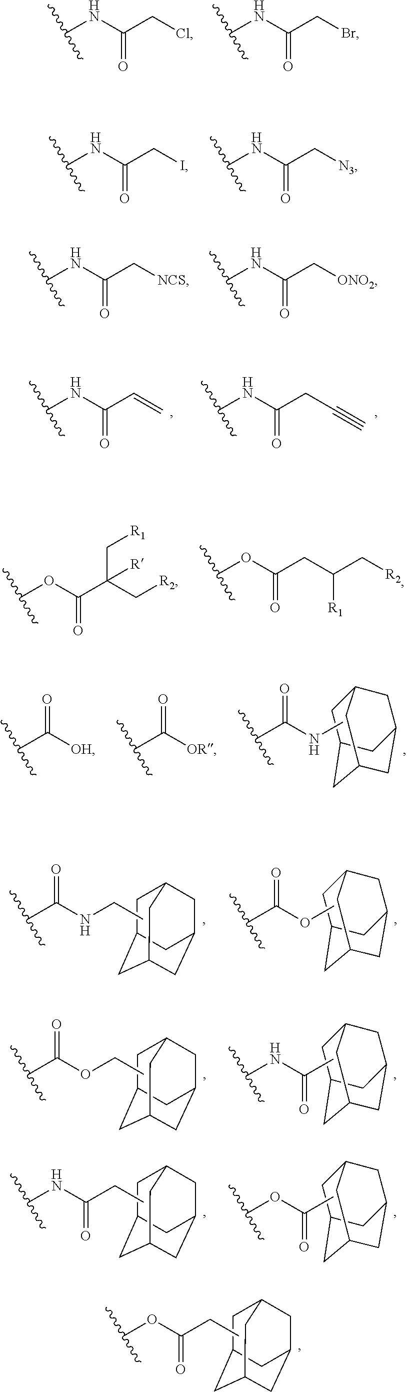
j is an integer from 1 to about 5,
R₁ and R₂ are independently selected from —H, -D, —Cl, —Br, —F, —OH, -Me, —CHF₂, —CH₂F, —CF₃,
R′ is selected from —HC, -D and —CH₃,
R″ is -alkyl,
B₄ is selected from 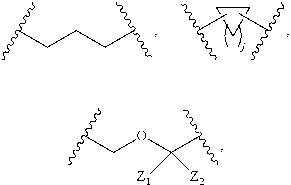
j is an integer from 1 to about 4,
Z₁, Z₂, independently selected from —H, -D, —F, -Me, —CF₃,
D₁, D₂, D₃ and D₄ are each independently selected from —CH₂—, —CF₂—, —CD₂- and 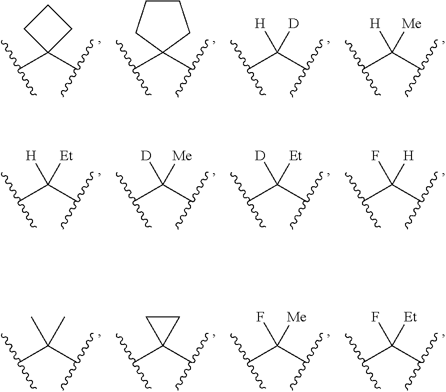
provided that at least one of D₁ or D₂ or D₃ or D₄ is a cycloalkyl group selected from: 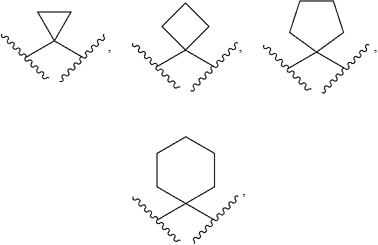 [SMILES: *C1(*)CC1 ; *C1(*)CCC1 ; *C1(*)CCCC1 ; *C1(*)CCCCC1]
Y₄ is selected from -Alkyl, —H, -D, —F, -L, —Cl, Br, —CF₃, —CN, —NCS, —N₃—NO₂, —SO₂F, —ONO₂, —OH, —SH, —NH₂, —(NH)-Alkyl, —N(Alkyl)₂, -heterocyclic ring, -carbocyclic ring, -Ph, —OPh, —SPh, —SOPh, —SO₂Ph and 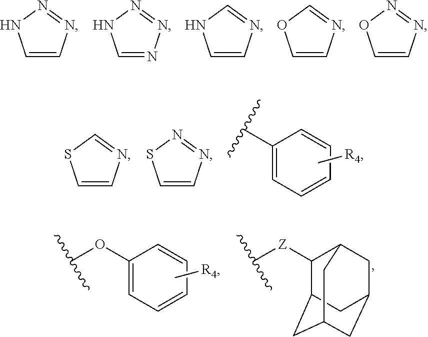
unless otherwise specifically denoted, each of the aforementioned groups is connected to the main structure from any possible position given that it is fulfilling the valency requirements,
R₄ is selected from —F, —CN, —CF₃,
Z is —NH—, —CH₂NH—, —NHCONH—, —COO—, —COOCH₂— or —CH₂—.

8. The compound of claim 7, wherein the compound is selected from the group consisting of:

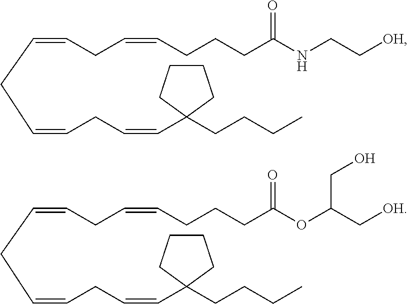

9. A compound of formula V or a pharmaceutically acceptable salt thereof, including all stereoisomers and enantiomers: 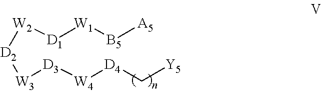
wherein:
A₅ is selected from 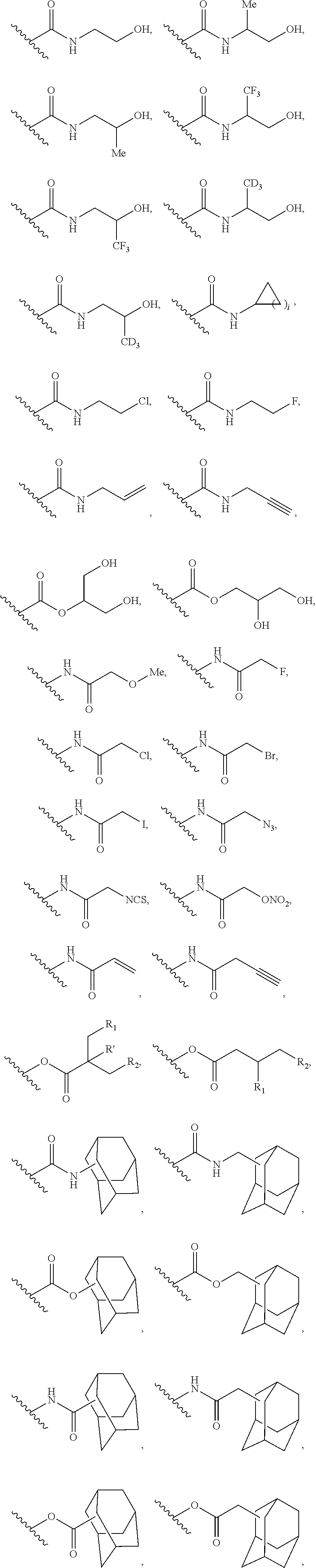
R₁, R₂ are independently selected from —H, -D, —Cl, —Br, —F, —OH, -Me, —CHF₂, —CH₂F, —CF₃,
R′ is selected from —H, -D and —CH₃,
R″ is -alkyl,
i is an integer from 1 to about 5,
B₅ is selected from 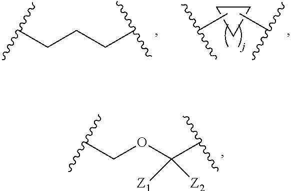
j is an integer from 1 to about 4,
Z₁, Z₂, independently selected from —H, -D, —F, -Me, —CF₃,
W₁, W₂, W₃ and W₄ are independently selected from ═, ≡ or a π electron system selected from the groups shown below 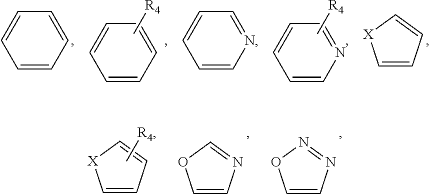
provided that the general structure V encompasses at least one of the aforementioned π electron systems and each of the π electron systems is connected to the main structure in any possible position given that it is fulfilling the valency requirements,
R₄ is selected from -D, —F, —CF₃, —CN,
X is selected from CH₂, NH, O, S,
D₁, D₂, D₃ and D₄ are independently selected from —CH₂—, —CF₂—, —CD₂- and 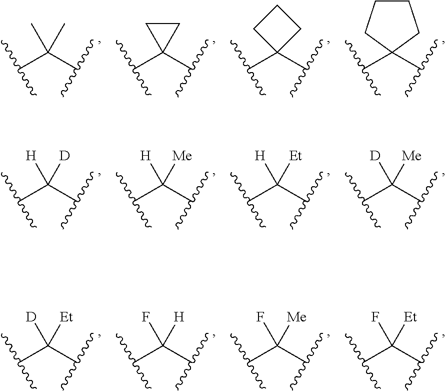
n is an integer from 1 to about 7,
Y₅ is selected from -Alkyl, —H, -D, —F, —I, —Cl, Br, —CF₃, —CN, —NCS, —N₃—NO₂, —SO₂F, —ONO₂, —OH, —SH, —NH₂, —(NH)-Alkyl, —N(Alkyl)₂, -heterocyclic ring, -carbocyclic ring, -Ph, —OPh, —SPh, —SOPh, —SO₂Ph and 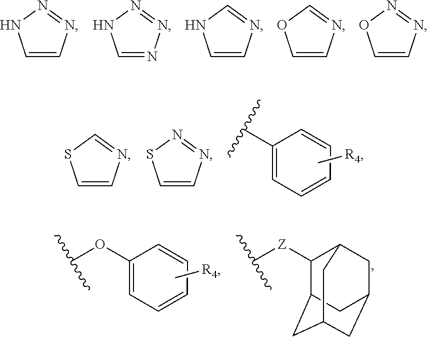
unless otherwise specifically denoted, each of the aforementioned groups is connected to the main structure from any possible position given that it is fulfilling the valency requirements,
R₄ is selected from —F, —CN, —CF₃,
Z is —NH—, —CH₂NH—, —NHCONH—, —COO—, —COOCH₂— or —CH₂—.

10. The compound of claim 9, wherein the compound is selected from the group consisting of:

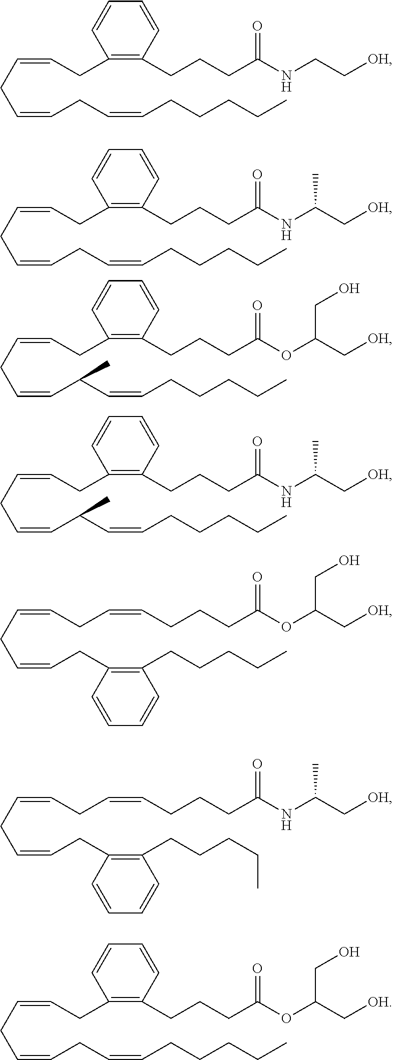

11. A pharmaceutical composition comprising a therapeutically effective amount of a compound of claim 1 or a pharmaceutically acceptable salt thereof and a pharmaceutically acceptable carrier.

12. A compound of claim 1, or a pharmaceutically acceptable salt thereof, wherein one or more atoms on the compound is in radioactive isotopic form.

13. A method of imaging or treating a subject in need thereof comprising administering an effective amount of a compound of claim 12, or a pharmaceutically acceptable salt thereof, to the subject.

14. A method of treating a condition in a subject in need thereof, the method comprising: administering to the subject a therapeutically effective amount of a compound or pharmaceutically acceptable salt of claim 1; wherein the condition is selected from the group consisting of pain; central pain; peripheral pain; neuropathic pain; neuropathy; inflammatory pain; neurodegenerative diseases including multiple sclerosis, Parkinson's disease, Huntington's chorea, Alzheimer's disease and amyotrophic lateral sclerosis; mental disorders such as schizophrenia and depression; mood disorders; addiction disorders; memory disorders; gastrointestinal motility disorders such as irritable bowel syndrome and diarrhea; dyskinesia; migraine; osteoporosis, osteoarthritis; high blood pressure disease or hypertension; peripheral vascular disease; coronary artery disease; abnormal heart rate; cardiac insufficiency; pulmonary hypertension; ocular hypertension or glaucoma; endotoxic shock; hypotensive shock; appetite disorders; immune system disorders; fertility disorders; diseases associated with motor dysfunction such as Tourette's syndrome; inflammation; neurological disorders; epilepsy; nausea; AIDS wasting syndrome; HIV and HIV related disorders; autism and autism spectrum disorder; cancer.

15. The method of claim 14, wherein the condition is selected from the group consisting of pain, central pain, and peripheral pain.

16. A method of stimulating a cannabinoid receptor in a subject, the method comprising: administering to the subject an effective amount of a compound or pharmaceutically acceptable salt of claim 1.

17. The method of claim 16, wherein the compound is a cannabinoid receptor agonist or a cannabinoid receptor partial agonist.

18. (canceled)

19. A method of selectively stimulating a CB1 cannabinoid receptor in subject, the method comprising: administering to the subject an effective amount of a compound or pharmaceutically acceptable salt of claim 1.

20. A method of selectively stimulating a cannabinoid receptor in the periphery of a subject, the method comprising: administering to the subject an effective amount of a compound or pharmaceutically acceptable salt of claim 1.

21. A method of assessing a compound as a CB1 modulator, CB2 modulator, or CB1/CB2 modulator, comprising contacting CB1, CB2 or both with the compound, and assessing an effect of the compound on CB1, CB2 or both, wherein the compound of claim 1, or a pharmaceutically acceptable salt thereof, optionally wherein the CB1, CB2, or CB1 and CB2 being contacted with the compound is on or in a cell, tissue, or organ.

22. (canceled)
